(19) United States
(12) Patent Application Publication
(10) Pub. No.: US 2011/0258736 A1
(43) Pub. Date: Oct. 20, 2011

(54) Pathogen Control Genes and Methods of Use in Plants

(75) Inventors: Piotr Puzio, Mariakerke (BE); Robert Ascenzi, Cary, NC (US); Volker Mittendorf, Hillsborough, NC (US)

(73) Assignee: BASF Plant Science GmbH

(21) Appl. No.: 12/674,916

(22) PCT Filed: Aug. 21, 2008
(86) PCT No.: PCT/EP08/60949

§ 371 Date: Feb. 24, 2010

Related U.S. Application Data
(60) Provisional application No. 60/969,211, filed on Aug. 31, 2007.
(60) Provisional application No. 60/969,190, filed on Aug. 31, 2007.

(51) Int. Cl. A01H 5/00; A01H 5/10; C12N 15/82

(52) U.S. Cl. 800/279; 435/320.1; 800/298; 800/320.1; 800/320.3; 800/320.2; 800/320; 800/312; 800/314; 800/317.3; 800/317.1; 800/306; 800/305; 800/278; 800/290

(57) ABSTRACT

This invention provides methods for conferring increased pathogen resistance to a plant. Specifically, the invention relates to methods of producing transgenic plants with increased nematode resistance, expression vectors comprising polynucleotides encoding polypeptides with anti-nematode activity, and transgenic plants and seeds generated thereof.

This application claims priority benefit of U.S. provisional patent application Ser. No. 60/969,190, filed Aug. 31, 2007, and Ser. No. 60/969,211, filed Aug. 31, 2007.

The invention relates to the control of pathogens. Disclosed herein are methods of producing transgenic plants with increased pathogen resistance, expression vectors comprising polynucleotides encoding for functional proteins, and transgenic plants and seeds generated thereof.

### BACKGROUND

One of the major goals of plant biotechnology is the generation of plants with advantageous novel properties, for example, to increase agricultural productivity, to increase quality in the case of foodstuffs, or to produce specific chemicals or pharmaceuticals. The plant's natural defense mechanisms against pathogens are frequently insufficient. The introduction of foreign genes from plants, animals or microbial sources can increase the defense.

A large group of plant pathogens of agro-economical importance are nematodes. Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots Infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. However, these patents do not provide any specific nematode genes that are useful for conferring resistance to nematode infection.

Despite several advances in some fields of plant biotechnology, success in achieving a pathogen resistance in plants has been very limited. Yield losses due to pathogens, in particular as a result of nematode attack, are a serious problem. Current practice to reduce nematode infestation is limited primarily to an intensive application of nematicides. Therefore, there is a need to identify safe and effective compositions and methods for controlling plant pathogens, in particular nematodes, and for the production of plants having increased resistance to plant pathogens, and ultimately for the increased yield.

### SUMMARY OF THE INVENTION

The present invention fulfills the need for plants that are nematode resistant, and concomitantly, demonstrate increased yield. The transgenic plants of the present invention comprise microbial genes that confer the phenotype of increased pathogen resistance when expressed in the plant.

In a first embodiment, the invention provides a nematode resistant transgenic plant transformed with an expression vector for over-expression comprising an isolated polynucleotide, selected from the group consisting of: (a) a polynucleotide having a sequence as defined in SEQ ID NO: 1, 3, 5, 7, 9, 135, 137, 139, 141, 143, 145, 147, 149, 151, 153, 155, 157, 159, or 161; (b) a polynucleotide encoding a polypeptide having a sequence as defined in SEQ ID NO:2, 4, 6, 8, 10, 136, 138, 140, 142, 144, 146, 148, 150, 152, 154, 156, 158, 160, or 162; (c) a polynucleotide having 70% sequence identity to a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; (d) a polynucleotide encoding a polypeptide having 70% sequence identity to a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant; (e) a polynucleotide hybridizing under stringent conditions to a polynucleotide comprising a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; (f) a polynucleotide hybridizing under stringent conditions to a polynucleotide comprising a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant.

In another embodiment, the invention provides a seed which is true breeding for a transgene comprising a polynucleotide that confers increased pathogen resistance to the plant grown from the seed, wherein the polynucleotide is selected from the group consisting of: (a) a polynucleotide having a sequence as defined in SEQ ID NO: 1, 3, 5, 7, 9, 135, 137, 139, 141, 143, 145, 147, 149, 151, 153, 155, 157, 159, or 161; (b) a polynucleotide encoding a polypeptide having a sequence as defined in SEQ ID NO:2, 4, 6, 8, 10, 136, 138, 140, 142, 144, 146, 148, 150, 152, 154, 156, 158, 160, or 162; (c) a polynucleotide having 70% sequence identity to a polynucleotide having a sequence as defined in SEQ ID NO: 1, 3, 5, 7, 9, 135, 137, 139, 141, 143, 145, 147, 149, 151, 153, 155, 157, 159, or 161; (d) a polynucleotide encoding a polypeptide having 70% sequence identity to a polypeptide having a sequence as defined in SEQ ID NO: 2, 4, 6, 8, 10, 136, 138, 140, 142, 144, 146, 148, 150, 152, 154, 156, 158, 160, or 162; (e) a polynucleotide hybridizing under stringent conditions to a polynucleotide comprising a polynucleotide having a sequence as defined in SEQ ID NO: 1, 3, 5, 7, 9, 135, 137, 139, 141, 143, 145, 147, 149, 151, 153, 155, 157, 159, or 161; (f) a polynucleotide hybridizing under stringent conditions to a polynucleotide comprising a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162.

In another embodiment, the invention provides an expression vector comprising a transcription regulatory element operably linked to a polynucleotide selected from the group consisting of: (a) a polynucleotide having a sequence as defined in SEQ ID NO: 1, 3, 5, 7, 9, 135, 137, 139, 141, 143, 145, 147, 149, 151, 153, 155, 157, 159, or 161; (b) a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162; (c) a polynucleotide having 70% sequence identity to a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; (d) a polynucleotide encoding a polypeptide having 70% sequence identity to a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant; (e) a polynucleotide hybridizing under stringent conditions to a polynucleotide comprising a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; and; (f) a polynucleotide hybridizing under stringent conditions to a polynucleotide under stringent conditions to a polynucleotide comprising a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant.

Another embodiment of the invention encompasses a method of producing a transgenic plant comprising a polynucleotide, wherein expression of the polynucleotide in the plant results in the plant demonstrating increased resistance to a pathogen as compared to a wild type control plant, and wherein the method comprises the steps of: 1) introducing into the plant an expression vector comprising a transcription regulatory element operably linked to a polynucleotide selected from the group consisting of: a) a polynucleotide having a sequence as defined in SEQ ID NO: 1, 3, 5, 7, 9, 135, 137, 139, 141, 143, 145, 147, 149, 151, 153, 155, 157, 159, or 161; b) a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162; c) a polynucleotide having 70% sequence identity to a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; d) a polynucleotide encoding a polypeptide having 70% sequence identity to a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant; e) a polynucleotide hybridizing under stringent conditions to a polynucleotide comprising a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; and f) a polynucleotide hybridizing under stringent conditions to a polynucleotide comprising a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant; and 2) selecting transgenic plants for increased pathogen resistance.

In another embodiment, the invention provides a method of increasing root growth in a crop plant, the method comprising the steps of transforming a crop plant cell with an expression vector comprising a polynucleotide selected from the group consisting of a polynucleotide having a sequence as defined in SEQ ID NO:9, 147, or 149 and a polynucleotide encoding a polypeptide having a sequence as defined in SEQ ID NO:10, 148, or 150 and selecting transgenic plants having increased root growth.

### BRIEF DESCRIPTION OF THE DRAWINGS

FIG. 1 shows a table describing the constitutively overexpressed gene ID and the associated secondary screen line number, SEQ ID NOs, and bioassay data Figure number.

FIG. 2a shows the decreased root-nematode infestation rate observed in line 99 overexpressing the E. coli gene b4225. The table includes the raw data for the plants tested for both the MC24 control and line 99. FIG. 2b shows average cyst count with bars indicating the standard error of the mean.

FIG. 3a shows the decreased root-nematode infestation rate observed in lines 219 overexpressing the yeast gene YKR043c. The table includes the raw data for the plants tested for both the MC24 control and line 219. FIG. 3b shows average cyst count with bars indicating the standard error of the mean.

FIG. 4a shows the decreased root-nematode infestation rate observed in lines 233 overexpressing the yeast gene YKR043c. The table includes the raw data for the plants tested for both the MC24 control and line 233. FIG. 4b shows average cyst count with bars indicating the standard error of the mean.

FIG. 5a shows the decreased root-nematode infestation rate observed in lines 234 overexpressing the yeast gene YKR043c. The table includes the raw data for the plants tested for both the MC24 control and line 234. FIG. 5b shows average cyst count with bars indicating the standard error of the mean.

FIG. 6a shows the decreased root-nematode infestation rate observed in line 285 overexpressing the E. coli gene b2796. The table includes the raw data for the plants tested for both the MC24 control and line 285. FIG. 6b shows average cyst count with bars indicating the standard error of the mean.

FIG. 7a shows the decreased root-nematode infestation rate observed in line 474 overexpressing the E. coli gene b0161. The table includes the raw data for the plants tested for both the MC24 control and line 474. FIG. 7b shows average cyst count with bars indicating the standard error of the mean.

FIG. 8a shows the decreased root-nematode infestation rate observed in line 75 overexpressing the yeast gene YGR256W. The table includes the raw data for the plants tested for both the MC24 control and line 75. FIG. 8b shows average cyst count with bars indicating the standard error of the mean.

FIGS. 9a and 9b shows a table of describing homologs of SEQ ID NOs 1 to 10. The corresponding homologs identified, homolog organism, homolog SEQ ID NOs, and homolog percent identity to the lead sequence is shown.

FIG. 10 shows a matrix table of homologs identified corresponding to SEQ ID NO:2 (b4225). The grey shaded cells indicate the SEQ ID NO of the corresponding amino acid sequence. The cells with no shading indicate the global amino acid percent Identity of the two SEQ ID NOs specific to the SEQ ID NOs that intersect on the x and y axis of the table in the corresponding cell.

FIG. 11 shows a matrix table of homologs identified corresponding to SEQ ID NO:4 (YKR043C). The grey shaded cells indicate the SEQ ID NO of the corresponding amino acid sequence. The cells with no shading indicate the global amino acid percent identity of the two SEQ ID NOs specific to the SEQ ID NOs that intersect on the x and y axis of the table in the corresponding cell.

FIG. 12 shows a matrix table of homologs identified corresponding to SEQ ID NO:6 (b2796). The grey shaded cells indicate the SEQ ID NO of the corresponding amino acid sequence. The cells with no shading indicate the global amino acid percent identity of the two SEQ ID NOs specific to the SEQ ID NOs that intersect on the x and y axis of the table in the corresponding cell.

FIG. 13 shows a matrix table of homologs identified corresponding to SEQ ID NO:8 (b0161). The grey shaded cells indicate the SEQ ID NO of the corresponding amino acid sequence. The cells with no shading indicate the global amino acid percent identity of the two SEQ ID NOs specific to the SEQ ID NOs that intersect on the x and y axis of the table in the corresponding cell.

FIG. 14 shows a matrix table of homologs identified corresponding to SEQ ID NO:10 (YGR256W). The grey shaded cells indicate the SEQ ID NO of the corresponding amino acid sequence. The cells with no shading indicate the global amino acid percent identity of the two SEQ ID NOs specific to the SEQ ID NOs that intersect on the x and y axis of the table in the corresponding cell.

FIG. 15a shows the decreased root-nematode infestation rate observed in line 268 overexpressing the yeast gene YLR319c. The table includes raw cyst count data for the MC24 control and line 268 plants tested. FIG. 15b shows average cyst count with bars indicating the standard error of the mean.

FIG. 16a shows the decreased root-nematode infestation rate observed in line 71 overexpressing the yeast gene YKR013W. The table includes the raw data for the plants tested for both the MC24 control and line 71. FIG. 16b shows average cyst count with bars indicating the standard error of the mean.

FIG. 17a shows the decreased root-nematode infestation rate observed in line 102 overexpressing the E. coli gene b3994. The table includes the raw data for the plants tested for both the MC24 control and line 102. FIG. 17b shows average cyst count with bars indicating the standard error of the mean.

FIG. 18a shows the decreased root-nematode infestation rate observed in line 393 overexpressing the yeast gene YPL101W. The table includes the raw data for the plants tested for both the MC24 control and line 393. FIG. 18b shows average cyst count with bars indicating the standard error of the mean.

FIG. 19a shows the decreased root-nematode infestation rate observed in line 47 overexpressing the yeast gene YPR004C. The table includes the raw data for the plants tested for both the MC24 control and line 47. FIG. 19b shows average cyst count with bars indicating the standard error of the mean.

FIG. 20a shows the decreased root-nematode infestation rate observed in line 398 overexpressing the yeast gene YNL283C. The table includes the raw data for the plants tested for both the MC24 control and line 398. FIG. 20b shows average cyst count with bars indicating the standard error of the mean.

FIG. 21a shows the decreased root-nematode infestation rate observed in line 49 overexpressing the yeast gene YOL137W. The table includes the raw data for the plants tested for both the MC24 control and line 49. FIG. 21b shows average cyst count with bars indicating the standard error of the mean.

FIG. 22a shows the decreased root-nematode infestation rate observed in line 18 overexpressing the yeast gene YKL033W. The table includes the raw data for the plants tested for both the MC24 control and line 18. FIG. 22b shows average cyst count with bars indicating the standard error of the mean.

FIG. 23a shows the decreased root-nematode infestation rate observed in line 266 overexpressing the yeast gene YNL249C. The table includes the raw data for the plants tested for both the MC24 control and line 266. FIG. 23b shows average cyst count with bars indicating the standard error of the mean.

FIG. 24a shows the decreased root-nematode infestation rate observed in line 52 overexpressing the yeast gene YPL118W. The table includes the raw data for the plants tested for both the MC24 control and line 52. FIG. 24b shows average cyst count with bars indicating the standard error of the mean.

FIG. 25a shows the decreased root-nematode infestation rate observed in line 433 overexpressing the yeast gene YDR204W. The table includes the raw data for the plants tested for both the MC24 control and line 433. FIG. 25b shows average cyst count with bars indicating the standard error of the mean.

FIG. 26a shows the decreased root-nematode infestation rate observed in line 471 overexpressing the E. coli gene b0186. The table includes the raw data for the plants tested for both the MC24 control and line 471. FIG. 26b shows average cyst count with bars indicating the standard error of the mean.

FIG. 27a shows the decreased root-nematode infestation rate observed in line 91 overexpressing the E. coli gene b4349. The table includes the raw data for the plants tested for both the MC24 control and line 91. FIG. 27b shows average cyst count with bars indicating the standard error of the mean.

FIG. 28a shows the decreased root-nematode infestation rate observed in line 16 overexpressing the yeast gene YGR277c. The table includes the raw data for the plants tested for both the MC24 control and line 16. FIG. 28b shows average cyst count with bars indicating the standard error of the mean.

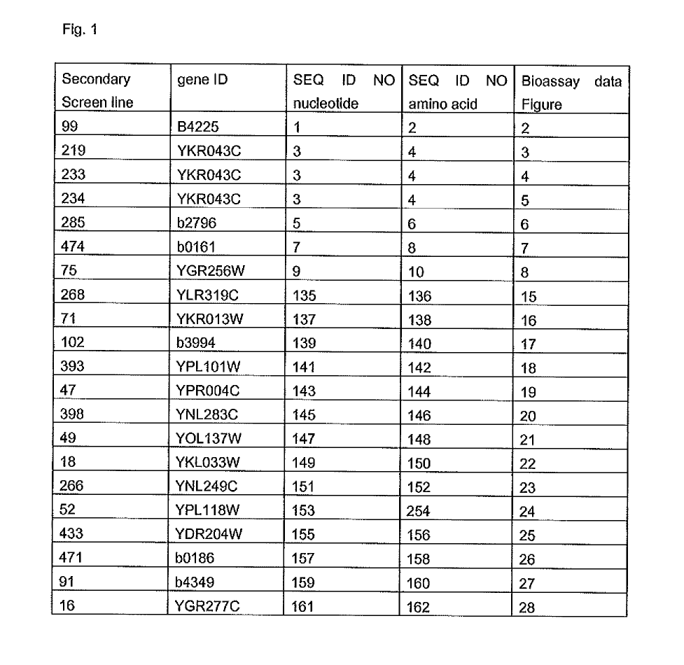

### DETAILED DESCRIPTION OF PREFERRED EMBODIMENTS

The present invention may be understood more readily by reference to the following detailed description and the examples included herein. However, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

Unless otherwise noted, the terms used herein are to be understood according to conventional usage by those of ordinary skill in molecular biology. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement).

Throughout this application, various publications are referenced. The disclosures of all of these publications and those references cited within those publications in their entireties are hereby incorporated by reference into this application in order to more fully describe the state of the art to which this invention pertains. A number of standard molecular biology techniques are described in Sambrook and Russell, 2001 Molecular Cloning, Third Edition, Cold Spring Harbor, Plainview, N.Y.; Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York.

As used herein and in the appended claims, the singular form “a”, “an”, or “the” includes plural reference unless the context clearly dictates otherwise. As used herein, the word “or” means any one member of a particular list and also Includes any combination of members of that list.

As used herein, the word “nucleic acid”, “nucleotide”, or “polynucleotide” is intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. A polynucleotide as defined herein can be single-stranded or double-stranded. Such nucleic acids or polynucleotides include, but are not limited to, coding sequences of structural genes, anti-sense sequences, and non-coding regulatory sequences that do not encode mRNAs or protein products.

As used herein, an “isolated” polynucleotide, preferably, is substantially free of other cellular materials or culture medium when produced by recombinant techniques, or substantially free of chemical precursors when chemically synthesized. The term “isolated”, however, also encompasses a polynucleotide present in a genomic locus other than its natural locus or a polypeptide present in its natural locus being genetically modified or exogenously (i.e. artificially) manipulated.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The term “operably linked” or “functionally linked” as used herein refers to the association of nucleic acid sequences on single nucleic acid fragment so that the function of one is affected by the other. For example, a regulatory DNA is said to be “operably linked to” a DNA that expresses an RNA or encodes a polypeptide if the two DNAs are situated such that the regulatory DNA affects the expression of the coding DNA.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription.

The term “transcription regulatory element” as used herein refers to a polynucleotide that is capable of regulating the transcription of an operably linked polynucleotide. It includes, but not limited to, promoters, enhancers, introns, 5′ UTRs, and 3′ UTRs.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises the left border and the right border and may include a gene of interest in between. The term “expression vector” as used herein means a vector capable of directing expression of a particular nucleotide in an appropriate host cell. An expression vector comprises a regulatory nucleic acid element operably linked to a nucleic acid of interest, which is—optionally—operably linked to a termination signal and/or other regulatory element.

The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs). Allelic variants are also encompassed in the definition of homologs as used herein.

As used herein, the term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

As used herein, “percentage of sequence identity” or “sequence identity percentage” denotes a value determined by first noting in two optimally aligned sequences over a comparison window, either globally or locally, at each constituent position as to whether the identical nucleic acid base or amino acid residue occurs in both sequences, denoted a match, or does not, denoted a mismatch. As said alignment are constructed by optimizing the number of matching bases, while concurrently allowing both for mismatches at any position and for the introduction of arbitrarily-sized gaps, or null or empty regions where to do so increases the significance or quality of the alignment, the calculation determines the total number of positions for which the match condition exists, and then divides this number by the total number of positions in the window of comparison, and lastly multiplies the result by 100 to yield the percentage of sequence identity. “Percentage of sequence similarity” for protein sequences can be calculated using the same principle, wherein the conservative substitution is calculated as a partial rather than a complete mismatch. Thus, for example, where an identical amino acid is given a score of 1 and a non-conservative substitution is given a score of zero, a conservative substitution is given a score between zero and 1. The scoring of conservative substitutions can be obtained from amino acid matrices known in the art, for example, Blosum or PAM matrices.

Methods of alignment of sequences for comparison are well known in the art. The determination of percent identity or percent similarity (for proteins) between two sequences can be accomplished using a mathematical algorithm. Preferred, non-limiting examples of such mathematical algorithms are, the algorithm of Myers and Miller (Bioinformatics, 4(1):11-17, 1988), the Needleman-Wunsch global alignment (J. Mol. Biol., 48(3):443-53, 1970), the Smith-Waterman local alignment (J. Mol. Biol., 147:195-197, 1981), the search-for-similarity-method of Pearson and Lipman (PNAS, 85(8): 2444-2448, 1988), the algorithm of Karlin and Altschul (Altschul et al., J. Mol. Biol., 215(3):403-410, 1990; PNAS, 90:5873-5877, 1993). Computer implementations of these mathematical algorithms are commercially available and can be used for comparison of sequences to determine sequence identity or to identify homologs. See, for example, Thompson et. al. Nucleic Acids Res. 22:4673-4680, 1994) as implemented in the Vector NTI package (Invitrogen, 1600 Faraday Ave., Carlsbad, Calif. 92008).

As used herein, the term “hybridizes under stringent conditions” is intended to describe conditions for hybridization and washing under which nucleotide sequences at least 60% similar or identical to each other typically remain hybridized to each other. In another embodiment, the conditions are such that sequences at least about 65%, or at least about 70%, or at least about 75% or more similar or identical to each other typically remain hybridized to each other. Such stringent conditions are known to those skilled in the art and described as below. A preferred, non-limiting example of stringent conditions are hybridization in 6× sodium chloride/sodium citrate (SSC) at about 45° C., followed by one or more washes in 0.2×SSC, 0.1% SDS at 50-65° C.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “tissue” with respect to a plant (or “plant tissue”) means arrangement of multiple plant cells, including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissues may be in planta, in organ culture, tissue culture, or cell culture.

The term “organ” with respect to a plant (or “plant organ”) means parts of a plant and may include, but not limited to, for example roots, fruits, shoots, stems, leaves, hypocotyls, cotyledons, anthers, sepals, petals, pollen, seeds, etc.

The term “plant” as used herein can, depending on context, be understood to refer to whole plants, plant cells, plant organs, plant seeds, and progeny of same. The word “plant” also refers to any plant, particularly, to seed plant, and may include, but not limited to, crop plants. Plant parts include, but are not limited to, stems, roots, shoots, fruits, ovules, stamens, leaves, embryos, meristematic regions, callus tissue, gametophytes, sporophytes, pollen, microspores, hypocotyls, cotyledons, anthers, sepals, petals, pollen, seeds and the like. The term “plant” as used herein can be monocotyledonous crop plants, such as, for example, cereals including wheat, barley, sorghum, rye, triticale, maize, rice, sugarcane, and trees including apple, pear, quince, plum, cherry, peach, nectarine, apricot, papaya, mango, poplar, pine, sequoia, cedar, and oak. The term “plant” as used herein can be dicotyledonous crop plants, such as pea, alfalfa, soybean, carrot, celery, tomato, potato, cotton, tobacco, pepper, canola, oilseed rape, beet, cabbage, cauliflower, broccoli, lettuce and Arabidopsis thaliana. The class of plants that can be used in the method of the Invention is generally as broad as the class of higher and lower plants amenable to transformation techniques, including angiosperms (monocotyledonous and dicotyledonous plants), gymnosperms, ferns, horsetails, psilophytes, bryophytes, and multicellular algae. The plant can be from a genus selected from the group consisting of Medicago, Solanum, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium.

The term “transgenic” as used herein is intended to refer to cells and/or plants which contain a transgene, or whose genome has been altered by the introduction of at least one transgene, or that have incorporated exogenous genes or polynucleotides. Transgenic cells, tissues, organs and plants may be produced by several methods including the introduction of a “transgene” comprising at least one polynucleotide (usually DNA) into a target cell or integration of the transgene into a chromosome of a target cell by way of human intervention, such as by the methods described herein.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “null segregant” as used herein refers to a progeny (or lines derived from the progeny) of a transgenic plant that does not contain the transgene due to Mendelian segregation.

The term “wild type” as used herein refers to a plant cell, seed, plant component, plant tissue, plant organ, or whole plant that has not been genetically modified or treated in an experimental sense.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “syncytia site” as used herein refers to the feeding site formed in plant roots after nematode infestation. The site is used as a source of nutrients for the nematodes. A syncytium is the feeding site for cyst nematodes and giant cells are the feeding sites of root knot nematodes.

Crop plants and corresponding pathogenic nematodes are listed In Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). For example, plant parasitic nematodes that are targeted by the present invention include, without limitation, cyst nematodes and root-knot nematodes. Specific plant parasitic nematodes which are targeted by the present invention include, without limitation, Heterodera glycines, Heterodera schachtii, Heterodera avenae, Heterodera oryzae, Heterodera cajani, Heterodera trifolii, Globodera pallida, G. rostochiensis, or Globodera tabacum, Meloidogyne incognita, M. arenaria, M. hapla, M. javanica, M. naasi, M. exigua, Ditylenchus dipsaci, Ditylenchus angustus, Radopholus similis, Radopholus citrophilus, Helicotylenchus multicinctus, Pratylenchus coffeae, Pratylenchus brachyurus, Pratylenchus vulnus, Paratylenchus curvitatus, Paratylenchus zeae, Rotylenchulus reniformis, Paratrichodorus anemones, Paratrichodorus minor, Paratrichodorus christiei, Anguina tritici, Bidera avenae, Subanguina radicicola, Hoplolaimus seinhorsti, Hoplolaimus Columbus, Hoplolaimus galeatus, Tylenchulus semipenetrans, Hemicycliophora arenaria, Rhadinaphelenchus cocophilus, Belonolaimus longicaudatus, Trichodorus primitivus, Nacobbus aberrans, Aphelenchoides besseyi, Hemicriconemoides kanayaensis, Tylenchorhynchus claytoni, Xiphinema americanum, Cacopaurus pestis, and the like.

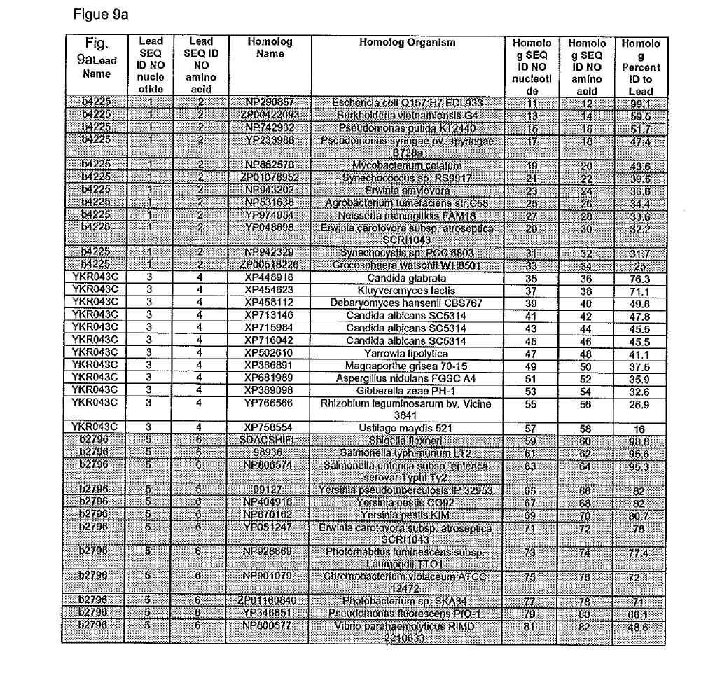

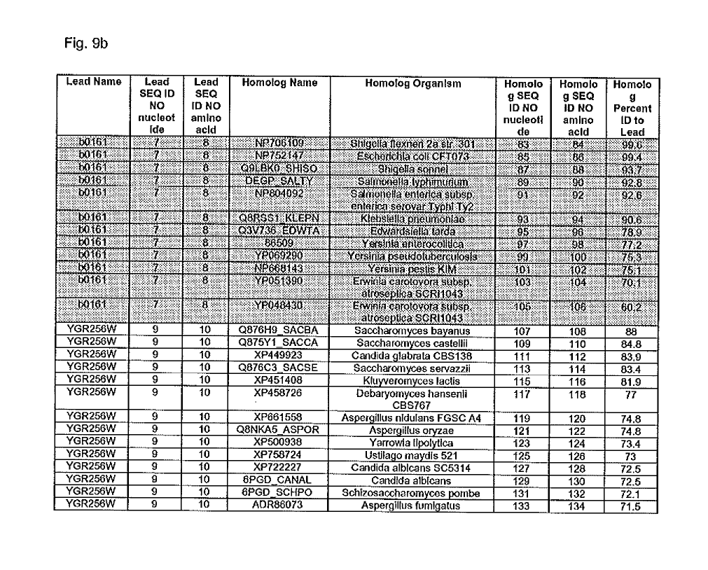

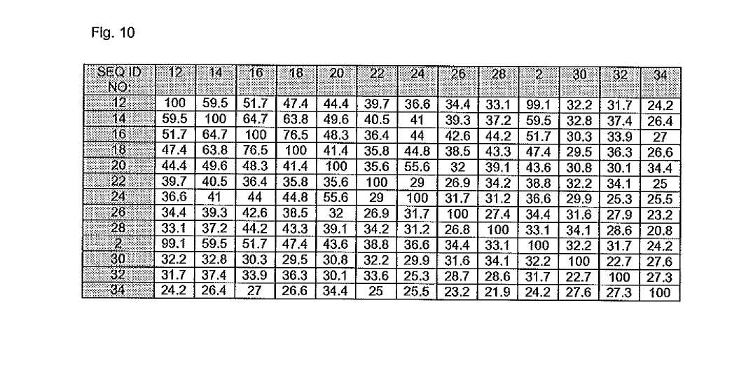

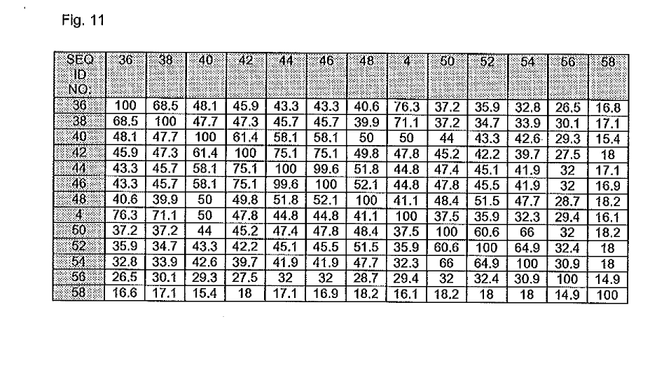

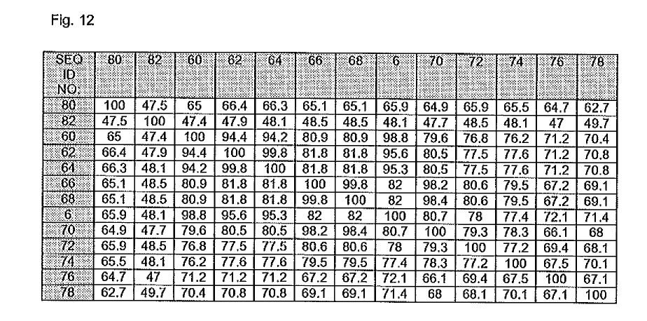

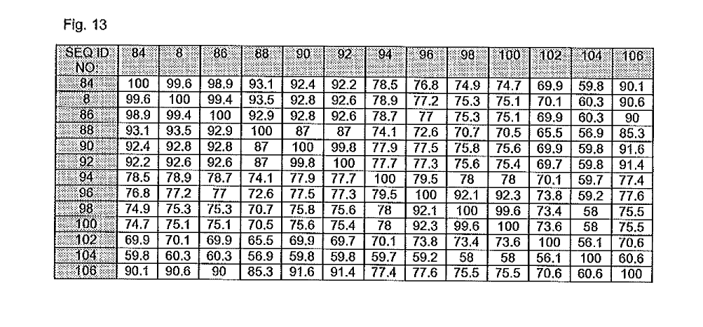

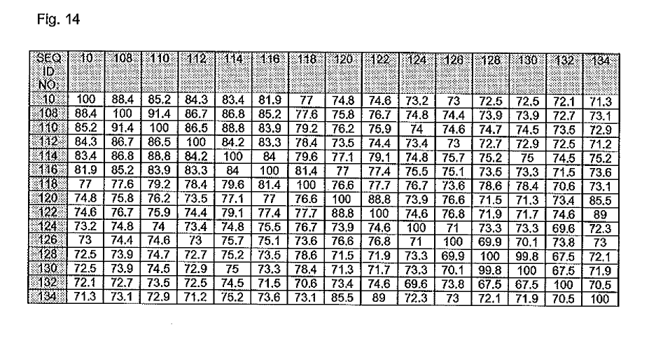

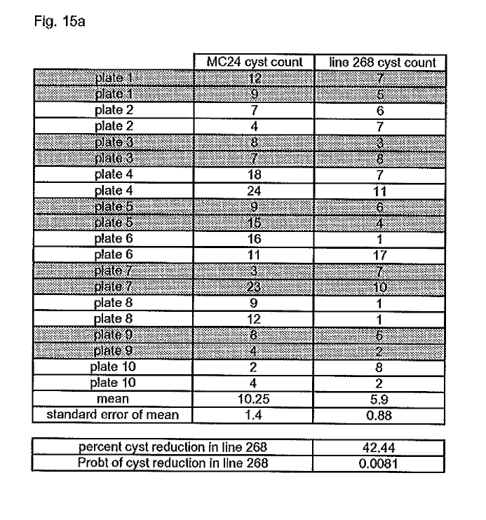

The first embodiment, the invention relates to a transgenic plant transformed with an expression vector comprising an isolated microbial polynucleotide capable of conferring increased nematode resistance to the plant. Exemplary microbial polynucleotide suitable for use in the Invention are set forth in SEQ ID NO: 1, 3, 5, 7, 9, 135, 137, 139, 141, 143, 145, 147, 149, 151, 153, 155, 157, 159, or 161. Alternatively, polynucleotides useful in the present invention may encode a polypeptide having a sequence as defined in SEQ ID NO: 2, 4, 6, 8, 10, 136, 138, 140, 142, 144, 146, 148, 150, 152, 154, 156, 158, 160, or 162. In yet another embodiment, a polynucleotide employed in the invention is at least about 50 to 60%, or at least about 60 to 70%, or at least about 70 to 80%, or at least about 80%, 81%, 82%, 83%, 84%, 86%, 87%, 88%, 89%, 90%, 91%, 92%, 93%, 94%, 95%, 96%, 97%, 98%, 99% or more identical or similar to a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant. In yet another embodiment, a polynucleotide employed in the invention comprises a polynucleotide encoding a polypeptide which is at least about 50 to 60%, or at least about 60 to 70%, or at least about 70 to 80%, or at least about 80%, 81%, 82%, 83%, 84%, 86%, 87%, 88%, 89%, 90%, 91%, 92%, 93%, 94%, 95%, 96%, 97%, 98%, 99% or more identical or similar to a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant. The invention may employ homologs of the polynucleotides of SEQ ID NO: 1, 3, 5, 7, 9, 135, 137, 139, 141, 143, 145, 147, 149, 151, 153, 155, 157, 159, or 161, and polynucleotides encoding homologs of the polypeptides of 2, 4, 6, 8, 10, 136, 138, 140, 142, 144, 146, 148, 150, 152, 154, 156, 158, 160, or 162. Exemplary homologs of the microbial polynucleotides suitable for use in the present invention are identified in FIGS. 9a and 9b.

In accordance with the invention, the plant may be a plant selected from the group consisting of monocotyledonous plants and dicotyledonous plants. The plant can be from a genus selected from the group consisting of maize, wheat, rice, barley, oat, rye, sorghum, banana, and ryegrass. The plant can be from a genus selected from the group consisting of pea, alfalfa, soybean, carrot, celery, tomato, potato, cotton, tobacco, pepper, oilseed rape, beet, cabbage, cauliflower, broccoli, lettuce and Arabidopsis thaliana.

The present invention also provides a transgenic seed which is true breeding for a polynucleotide described above, parts from the transgenic plant described above, and progeny plants from such a plant, including hybrids and inbreds. The invention also provides a method of plant breeding, e.g., to develop or propagate a crossed transgenic plant. The method comprises crossing a transgenic plant comprising a particular expression vector of the invention with itself or with a second plant, e.g., one lacking the particular expression vector, and harvesting the resulting seed of a crossed plant whereby the harvested seed comprises the particular expression vector. The seed is then planted to obtain a crossed transgenic progeny plant. The plant may be a monocot or a dicot. The crossed transgenic progeny plant may have the particular expression vector inherited through a female parent or through a male parent. The second plant may be an inbred plant. The crossed transgenic plant may be an inbred or a hybrid. Also included within the present invention are seeds of any of these crossed transgenic plants and their progeny.

Another embodiment of the invention relates to an expression vector comprising one or more transcription regulatory elements operably linked to one or more polynucleotides described above, wherein expression of the polynucleotide confers increased pathogen resistance to a transgenic plant. In one embodiment, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but are not limited to, the 35S CaMV promoter from plant viruses (Franck et al., 1980 Cell 21:285-294), the Nos promoter (An G. at al, The Plant Cell 3:225-233, 1990), the ubiquitin promoter (Christensen et al., Plant Mol. Biol. 12:619-632, 1992 and 18:581-8, 1991), the MAS promoter (Velten et al., EMBO J. 3:2723-30, 1984), the maize H3 histone promoter (Lepetit et al., Mol. Gen. Genet. 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028).

In accordance with the Invention, the transcription regulatory element may be a regulated promoter. A “regulated promoter” refers to a promoter that directs gene expression not constitutively, but in a temporally and/or spatially manner, and Includes both tissue-specific and inducible promoters. Different promoters may direct the expression of a gene or regulatory element in different tissues or cell types, or at different stages of development, or in response to different environmental conditions.

A “tissue-specific promoter” or “tissue-preferred promoter” refers to a regulated promoter that is not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of sequence. Suitable promoters include the napin-gene promoter from rapeseed (U.S. Pat. No. 5,608,152), the USP-promoter from Vicia faba (Baeumlein et al., 1991 Mol Gen Genet. 225(3):459-67), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., 1992 Plant Journal, 2(2):233-9) as well as promoters conferring seed specific expression in monocot plants like maize, barley, wheat, rye, rice, etc. Suitable promoters to note are the Ipt2 or Ipt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Promoters suitable for preferential expression in plant root tissues include, for example, the promoter derived from corn nicotianamine synthase gene (US 20030131377) and rice RCC3 promoter (U.S. Ser. No. 11/075,113). Suitable promoter for preferential expression in plant green tissues include the promoters from genes such as maize aldolase gene FDA (US 20040216189), aldolase and pyruvate orthophosphate dikinase (PPDK) (Taniguchi et. al., Plant Cell Physiol. 41(1):42-48, 2000).

“Inducible promoters” refer to those regulated promoters that can be turned on in one or more cell types by an external stimulus, for example, a chemical, light, hormone, stress, or a pathogen such as nematodes. Chemically inducible promoters are especially suitable if gene expression is wanted to occur in a time specific manner. Examples of such promoters are a salicylic acid inducible promoter (WO 95/19443), a tetracycline inducible promoter (Gatz et al., 1992 Plant J. 2:397-404), the light-inducible promoter from the small subunit of Ribulose-1,5-bis-phosphate carboxylase (ssRUBISCO), and an ethanol inducible promoter (WO 93/21334). Also, suitable promoters responding to biotic or abiotic stress conditions are those such as the pathogen inducible PRP1-gene promoter (Ward et al., 1993 Plant. Mol. Biol. 22:361-366), the heat inducible hsp80-promoter from tomato (U.S. Pat. No. 5,187,267), cold inducible alpha-amylase promoter from potato (WO 96/12814), the drought-inducible promoter of maize (Busk et. al., Plant J. 11:1285-1295, 1997), the cold, drought, and high salt inducible promoter from potato (Kirch, Plant Mol. Biol. 33:897-909, 1997) or the RD29A promoter from Arabidopsis (Yamaguchi-Shinozalei et. al., Mol. Gen. Genet. 236:331-340, 1993), many cold inducible promoters such as cor15a promoter from Arabidopsis (Genbank Accession No U01377), blt101 and blt4.8 from barley (Genbank Accession Nos AJ310994 and U63993), wcs120 from wheat (Genbank Accession No AF031235), mlip15 from corn (Genbank Accession No D26563), bn115 from Brassica (Genbank Accession No U01377), and the wound-inducible pinII-promoter (European Patent No. 375091). Of particular interest in the present invention are syncytia site preferred, or nematode feeding site induced, promoters, including, but not limited to promoters from the Mtn3-like promoter disclosed in PCT/EP2008/051328, the Mtn21-like promoter disclosed in PCT/EP2007/051378, the peroxidase-like promoter disclosed in PCT/EP2007/064356, the trehalose-6-phosphate phosphatase-like promoter disclosed in PCT/EP2007/063761 and the At5g12170-like promoter disclosed in PCT/EP2008/051329, all of the forgoing applications are herein incorporated by reference.

Yet another embodiment of the invention relates to a method of producing a transgenic plant comprising a polynucleotide, wherein the method comprises the steps of: 1) introducing into the plant the expression vector comprising a polynucleotide described above, wherein expression of the polynucleotide confers increased pathogen resistance to the plant; and 2) selecting transgenic plants for increased pathogen resistance.

A variety of methods for introducing polynucleotides into the genome of plants and for the regeneration of plants from plant tissues or plant cells are known in, for example, Plant Molecular Biology and Biotechnology (CRC Press, Boca Raton, Fla.), chapter 6/7, pp. 71-119 (1993); White F F (1993) Vectors for Gene Transfer in Higher Plants; Transgenic Plants, vol. 1, Engineering and Utilization, Ed.: Kung and Wu R, Academic Press, 15-38; Jenes B et al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

Transformation may result in transient or stable transformation and expression. Although a nucleotide sequence of the present invention can be inserted into any plant and plant cell falling within these broad classes, it is particularly useful in crop plant cells.

Various tissues are suitable as starting material (explant) for the Agrobacterium-mediated transformation process including but not limited to callus (U.S. Pat. No. 5,591,616; EP-A1 604 662), immature embryos (EP-A1 672 752), pollen (U.S. Pat. No. 54,929,300), shoot apex (U.S. Pat. No. 5,164,310), or in planta transformation (U.S. Pat. No. 5,994,624). The method and material described herein can be combined with virtually all Agrobacterium mediated transformation methods known in the art. Preferred combinations include, but are not limited to, the following starting materials and methods:

The nucleotides of the present invention can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. Nos. 5,451,513, 5,545,817, 5,545,818, and 5,877,462 in WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., PNAS 87, 8526-8530, 1990; Staub et al., Plant Cell 4, 39-45, 1992). The presence of cloning sites between these markers allows creation of a plastid targeting vector for introduction of foreign genes (Staub et al. EMBO J. 12, 601-606, 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et at., PNAS 90, 913-917, 1993). Other selectable markers useful for plastid transformation are known in the art and encompassed within the scope of the invention.

The transgenic plants of the invention may be used in a method of controlling infestation of a crop by a plant pathogen, which comprises the step of growing said crop from seeds comprising an expression vector comprising one or more transcription regulatory elements operably linked to one or more polynucleotides that encode an agent toxic to said plant pathogen, wherein the expression vector is stably integrated into the genomes of the seeds.

### EXAMPLES

### Example 1

### Primary Screening of Arabidopsis Lines with Beet Cyst Nematode

Seeds from selected Arabidopsis lines containing a microbial gene to be tested were packaged in filter paper envelopes and given an arbitrary identifier and used for primary screening. Primary screening consisted of the following steps: 1) sterilization by chlorine gas, 2) growth on selective media; 3) transfer to assay plates; 4) inoculation of seedlings in assay plates with defined amount J2 larvae; 5) counting of J4 female nematodes and cysts and 6) analysis of results; and 7) selection of lead lines.

Sterilized seeds consisting of a population segregating for expression of a microbial test gene were grown on Petri dishes containing Murashige Skoog medium with the appropriate selection agent added (glufosinate (Bayer Crop Science Kansas City, Mo.), imazethapyr (BASF Corporation, RTP, NC); or kanamycin, depending on the marker gene present in the Arabidopsis line). The Petri dishes were placed at 4° C. for 72 hours and then transferred to a 22° C. growth chamber. After 10 days, seedlings were selected on the basis of size and color. Individual seedlings that did not contain the transgene (i.e. null segregants) were stunted and chlorotic. Individual seedlings containing the transgene designed to express a microbial test gene were green and had fully expanded cotyledons. These individuals were selected for transfer to assay plates.

Selected seedlings from were transferred to 12 well assay plates containing 0.2 strength Knop medium solidified with 0.8% Daishin agar (Sijmons et al 1991), and maintained in a 24° C. growth chamber for 10 days with a 16 h photoperiod. At least two plates containing controls were used for each set of inoculations.

Transferred seedlings were grown under the same conditions for 10 additional days and then Inoculated with a defined number (90-100) of sterilized Heterodera schachtii J2 larvae. Inoculated seedlings were maintained a growth chamber for an additional 28 days.

After 28 days, plates were removed observed under a dissecting scope. The numbers of mature females (J4 females and adult-stage cysts) were counted and results recorded. A root score of 1-5 was assigned to each inoculated seedling with 1 being small and 5 being large. In addition, high-resolution images were taken on the day of inoculation and the day of counting.

Recorded results were subjected to statistical analysis using a SAS software package (SAS, Cary, N.C.). Analysis of results revealed sets of lines within groups inoculated with a particular batch of nematodes that had lower (putative resistant lines) or higher (putative hyper-susceptible lines) female numbers. Lines with a lower number of mature females were selected from sets inoculated with nematode batches resulting in a mean value of 10 mature females per seedling.

### Example 2

### Validation Screening of Selected Arabidopsis Lines

Seeds from lead lines selected on the basis of primary screening were packaged in filter paper envelopes and given an arbitrary identifier and used in a validation assay (secondary screen). A validation assay consisted of the same steps as in Example 1 with the exceptions described as follows.

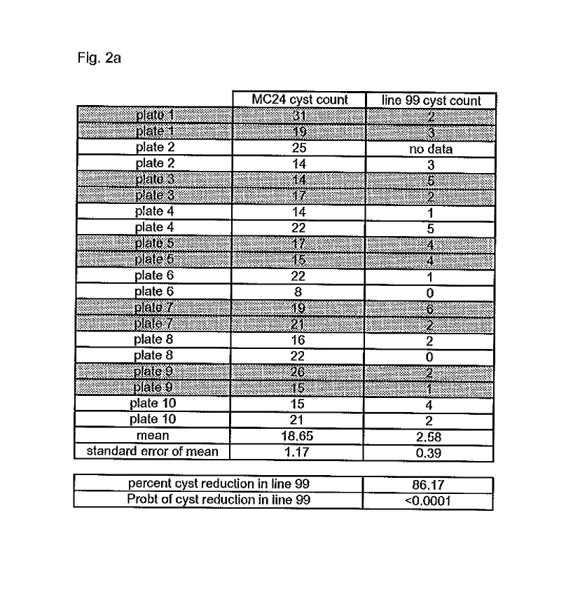

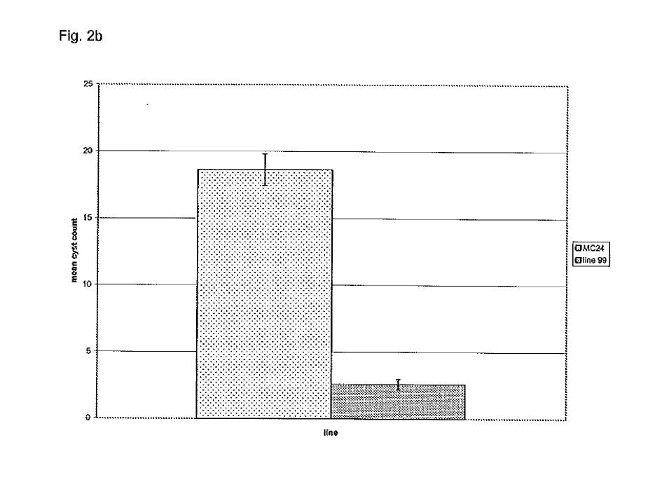

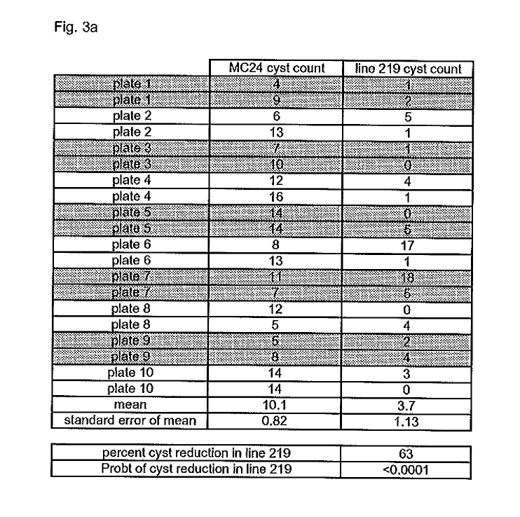

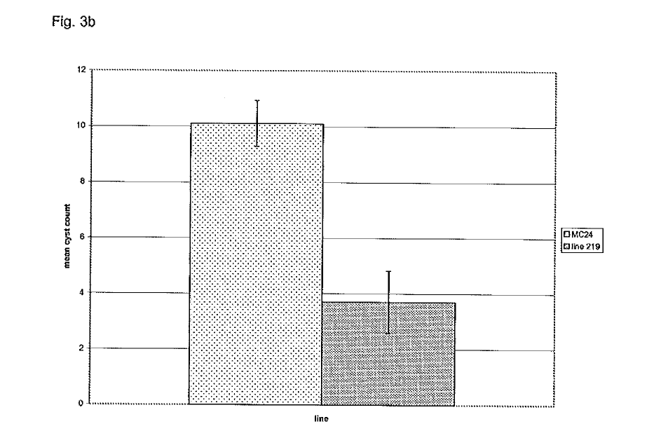

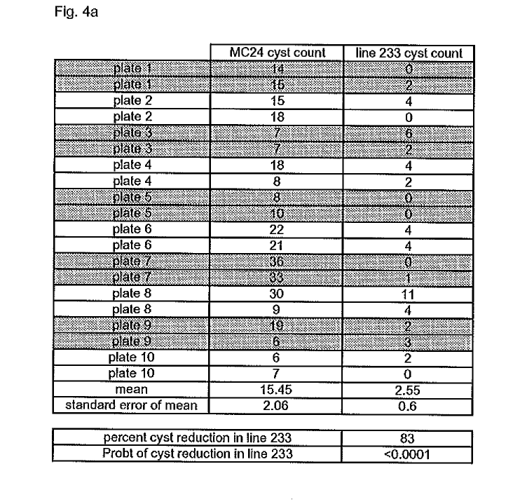

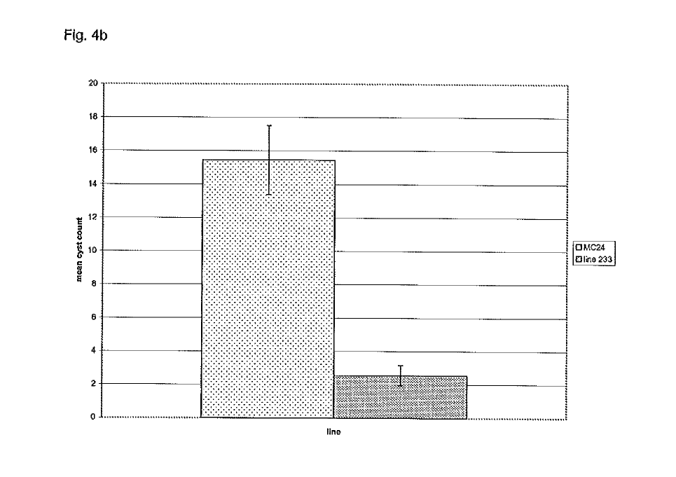

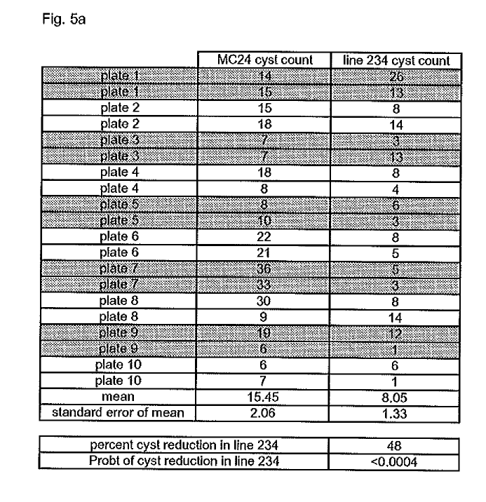

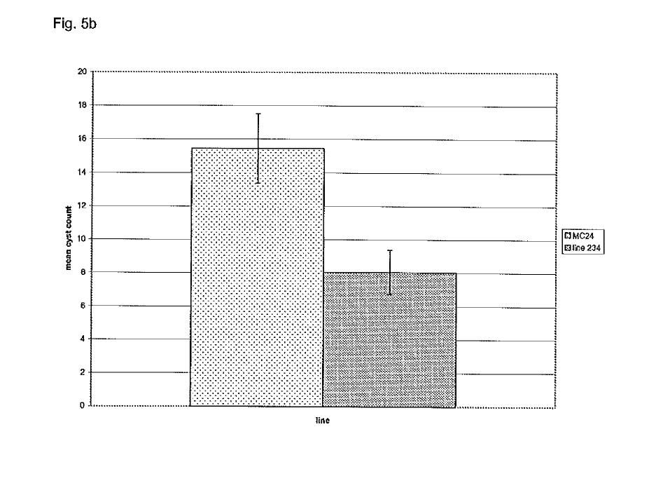

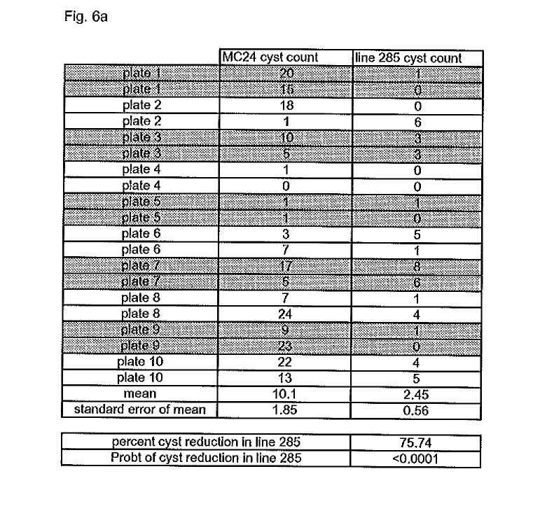

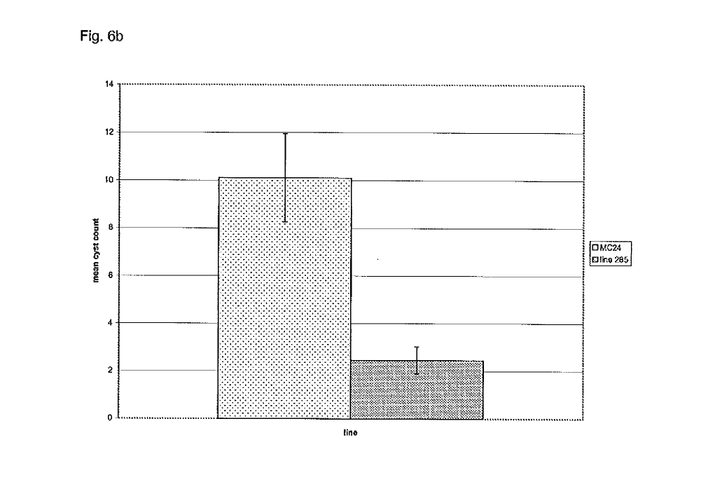

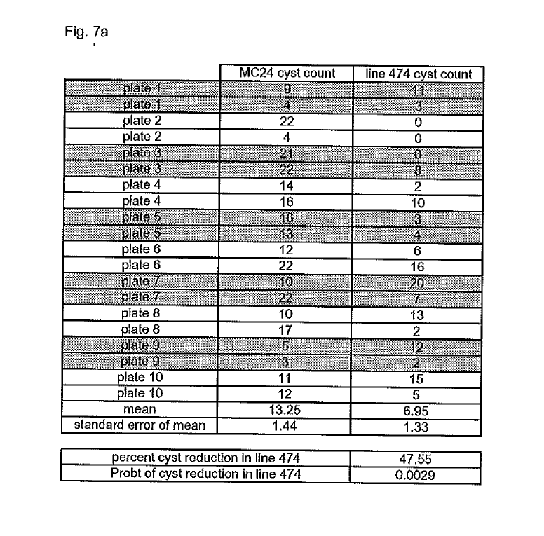

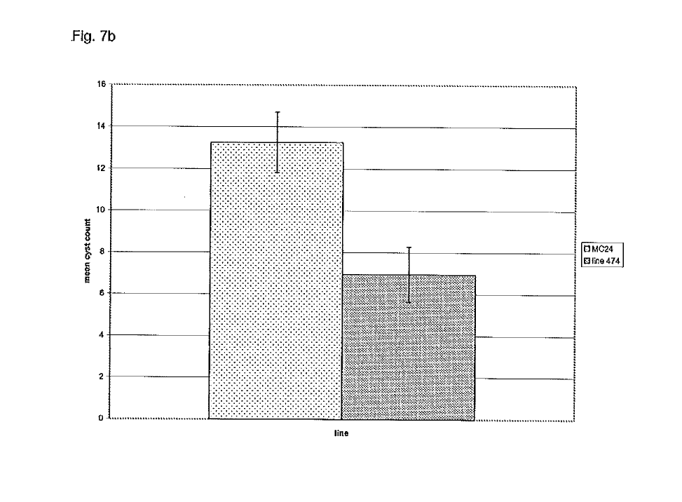

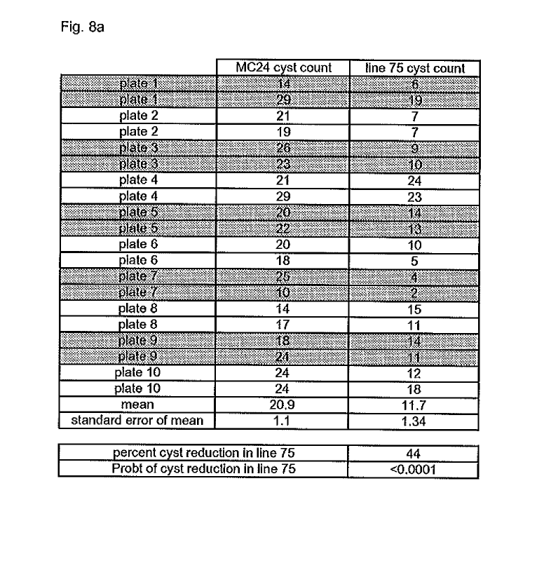

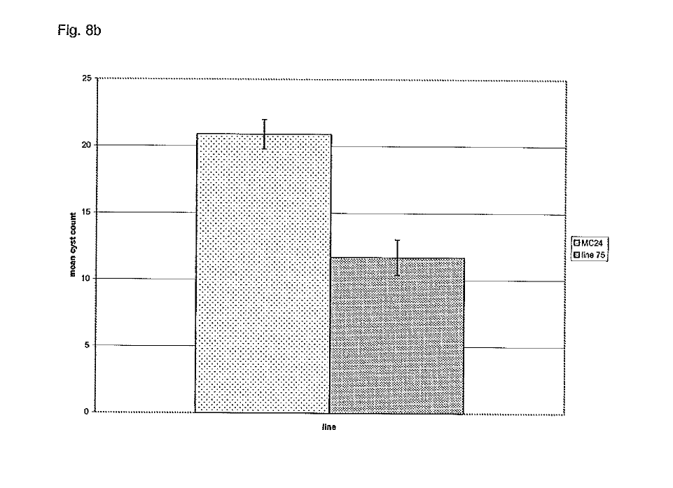

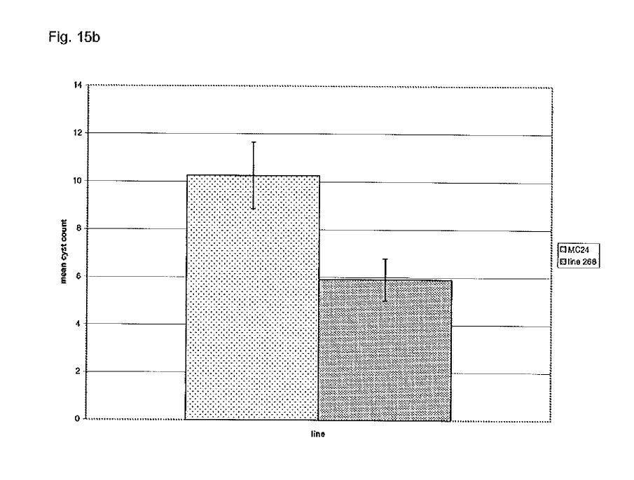

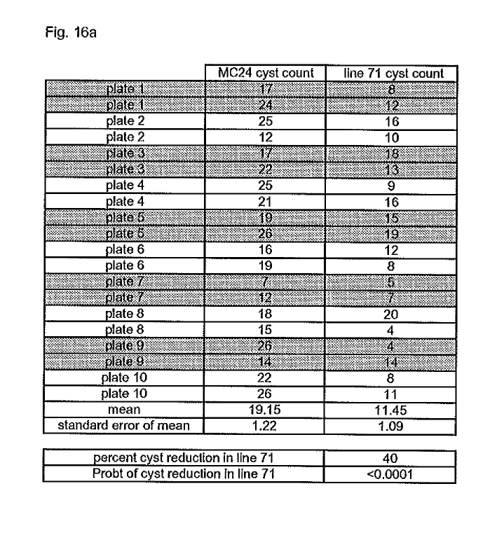

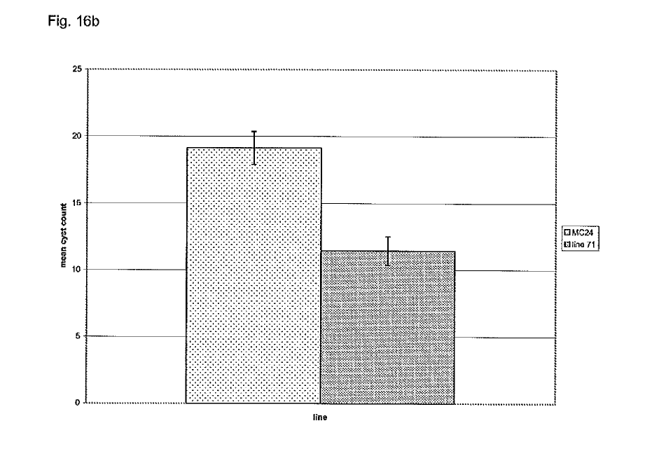

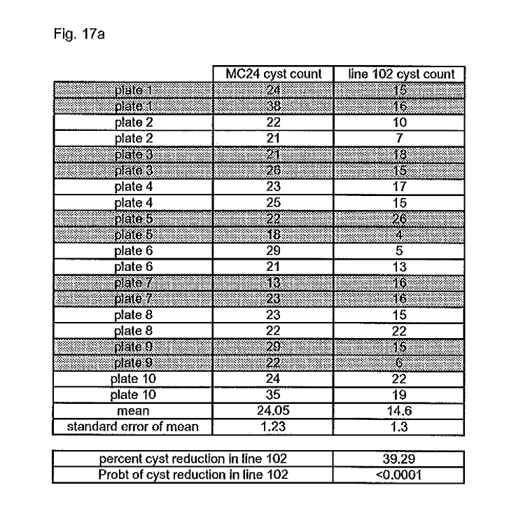

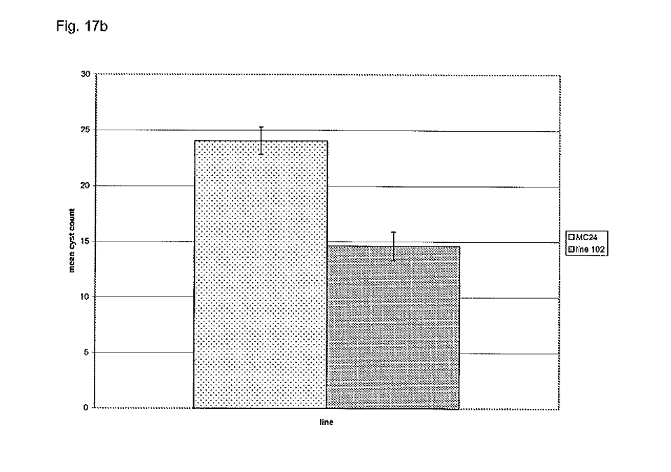

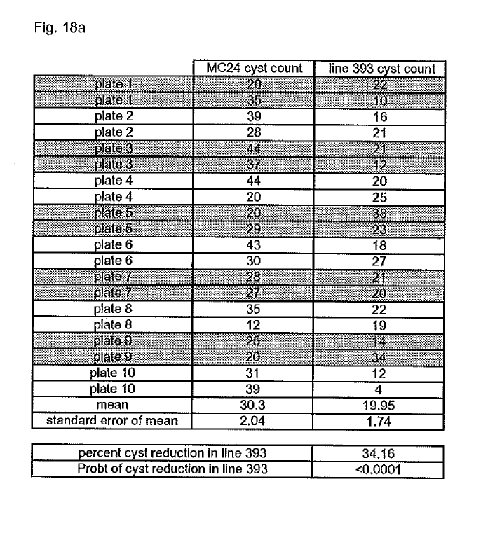

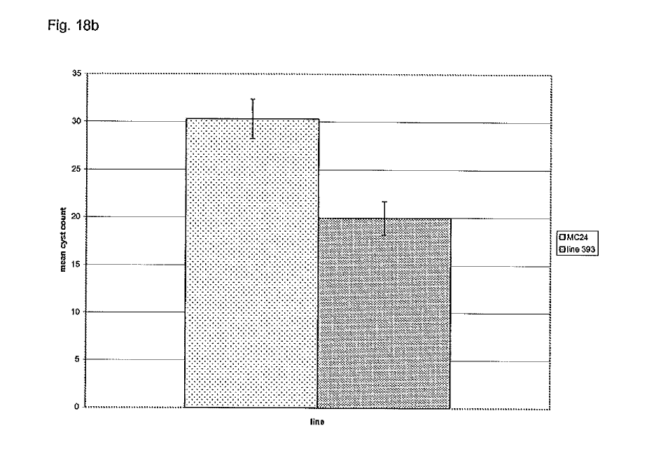

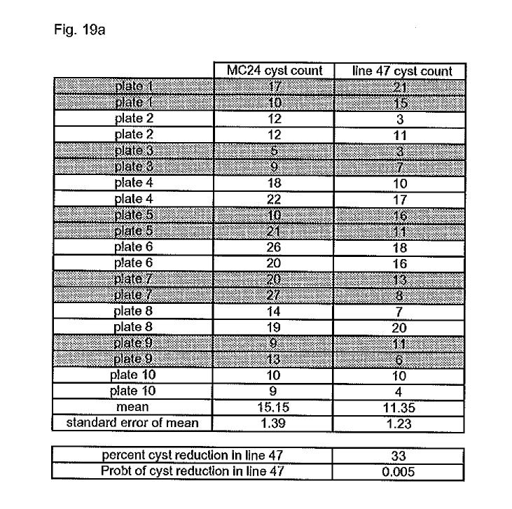

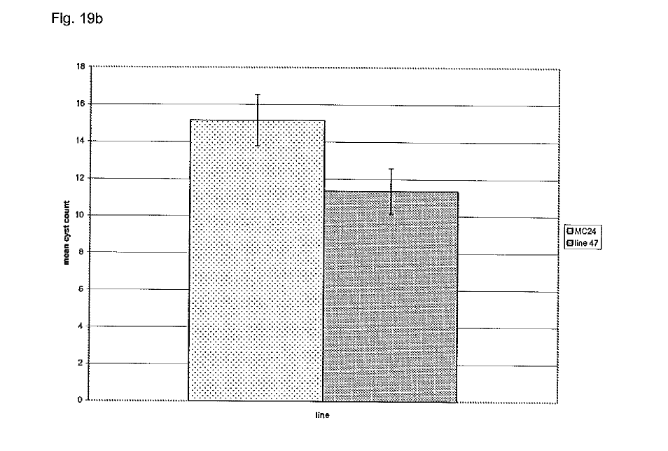

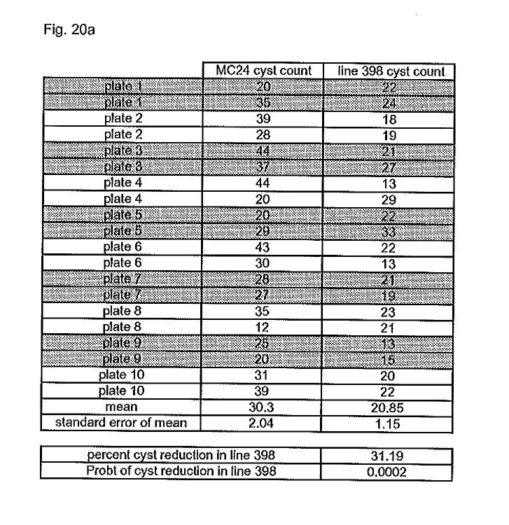

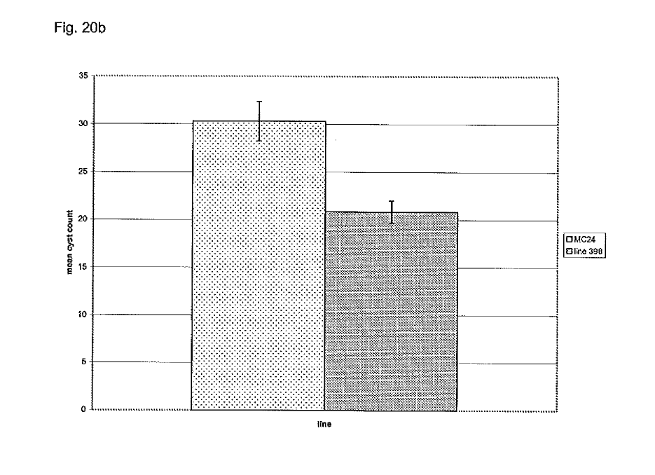

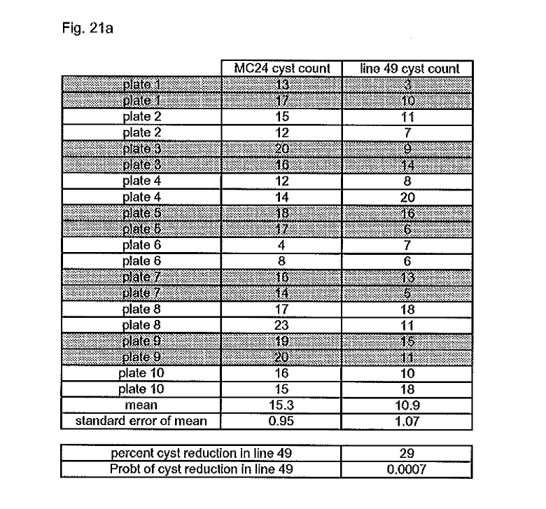

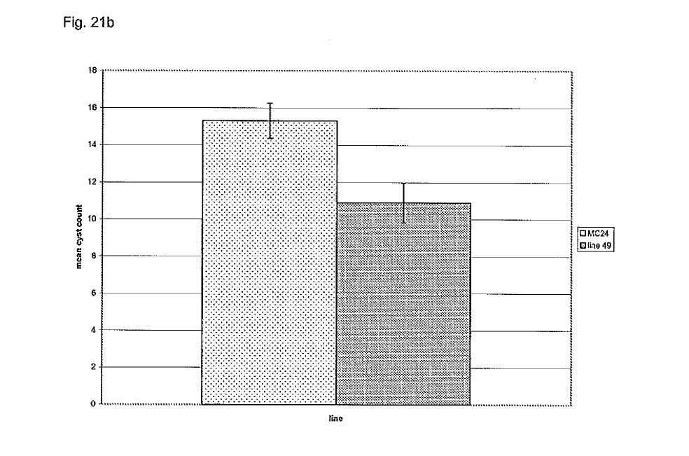

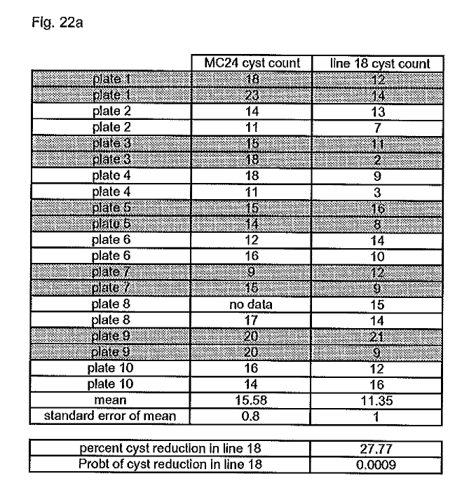

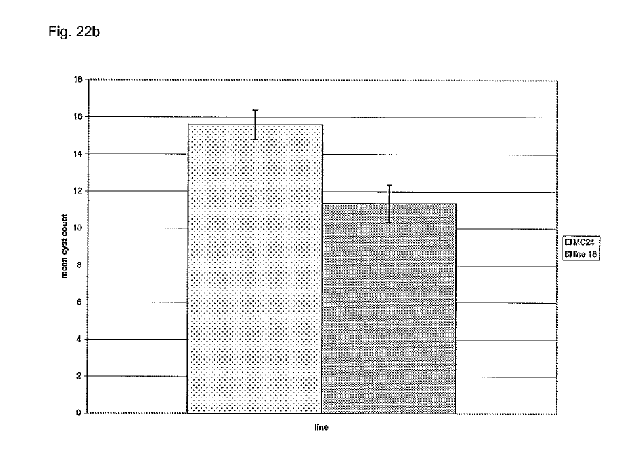

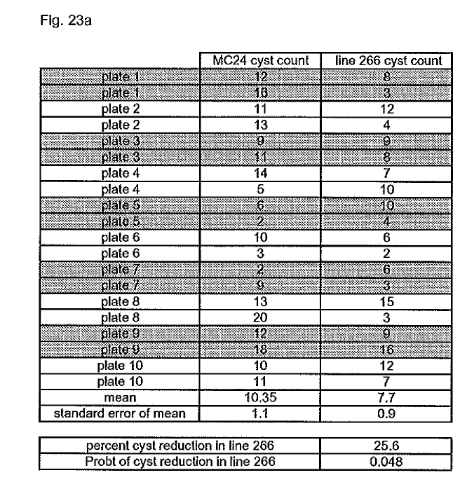

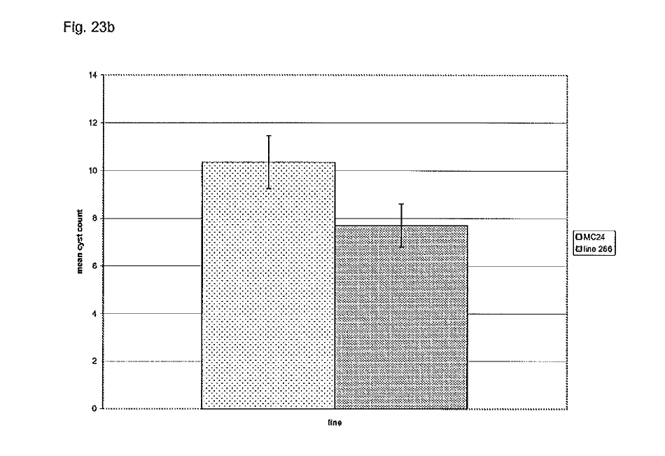

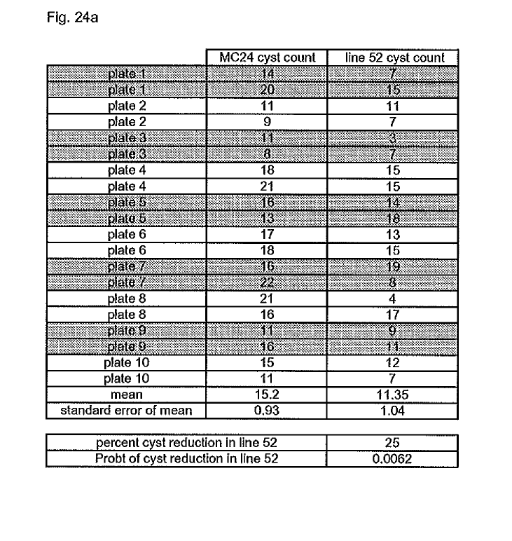

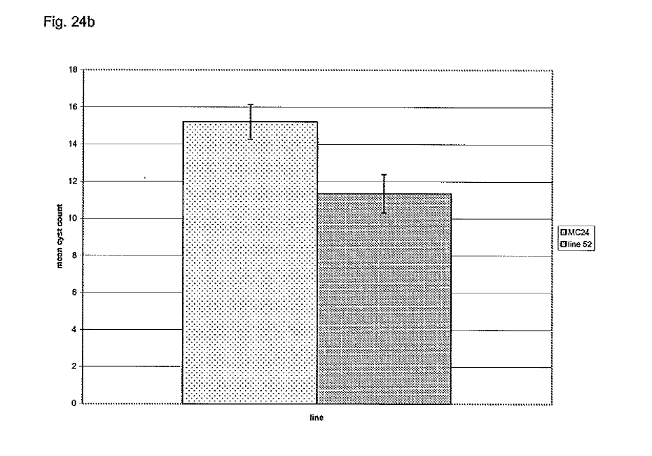

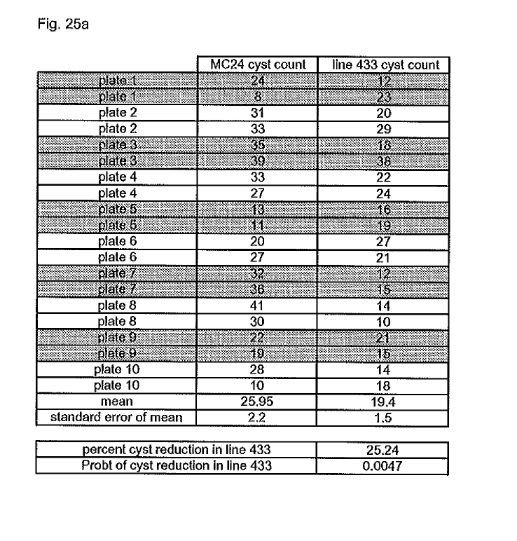

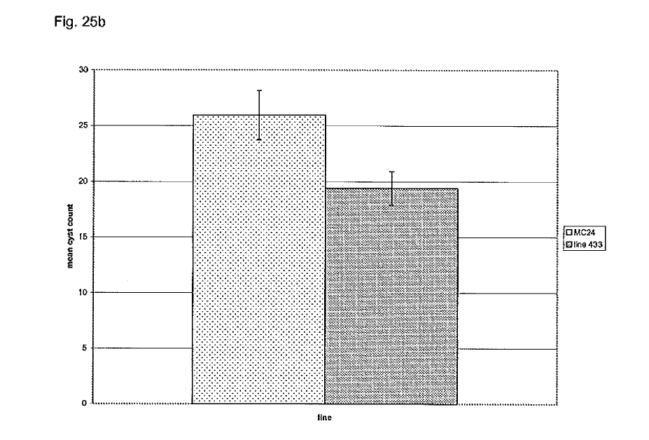

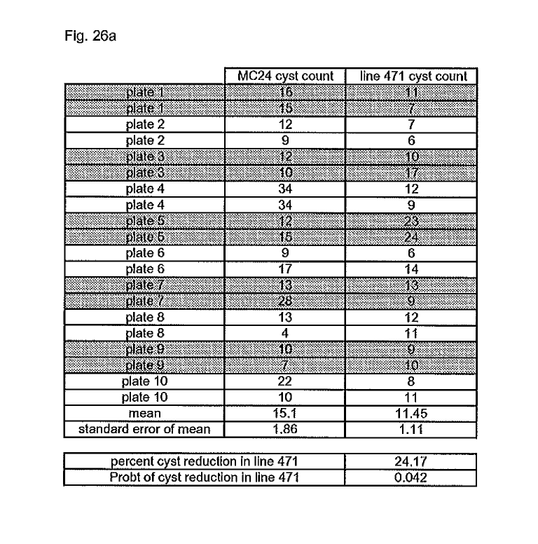

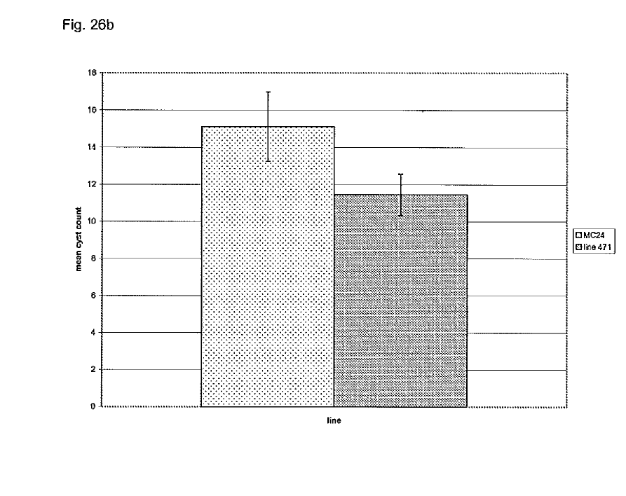

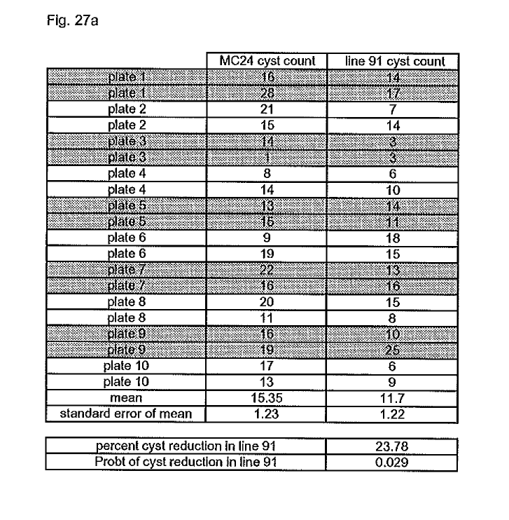

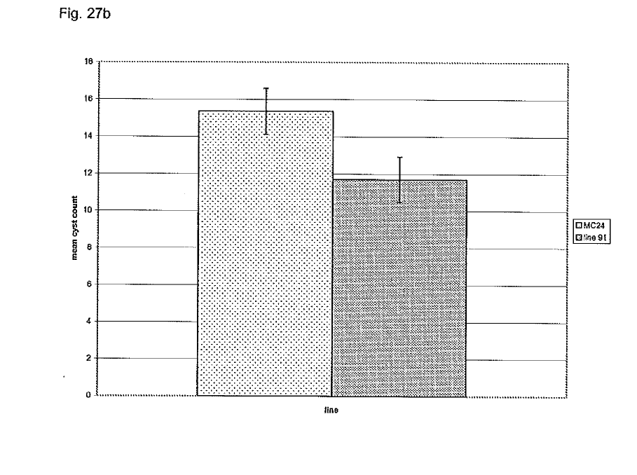

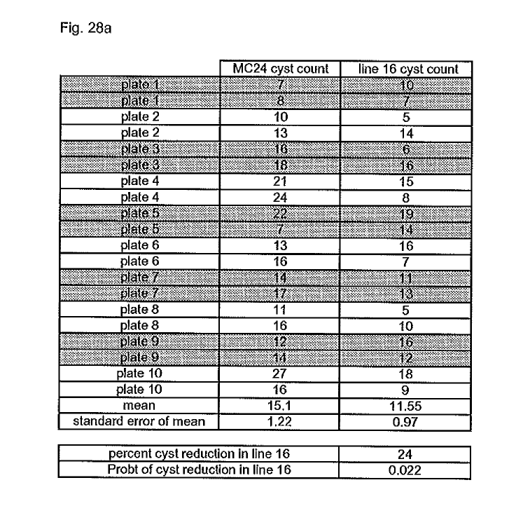

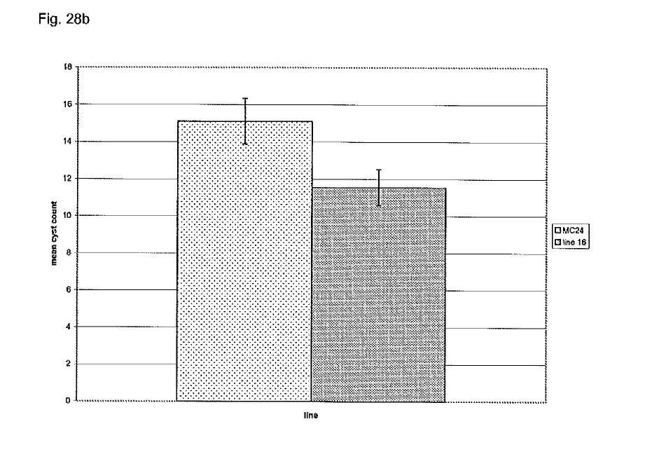

For the infection assay, 20 seedlings per line were transferred to 6-well plates containing Knop medium in order to allow greater root development relative to 12-well plates. Each plate contained two seedlings from a line and two controls. Thus, each plate contained two test lines and all replicates and corresponding controls for a given line were present on 10 plates. The seedlings were Inoculated with a greater number (250) of sterile J2 larvae relative to the first screen. These larvae were produced from in vitro root cultures and therefore the sterilization described in Example 1 was not necessary. Mature females were counted as described in the previous example and data analyzed by a t-test using the SAS software package (SAS, Cary, N.C.). Only those lines having corresponding controls averaging at least 20 J4 females per well, and showing a 25% difference from control plates with a p<0.05 were considered to be a validated lead. Cyst count data for validated leads overexpressing the sequences described by SEQ ID NO: 1, 3, 5, 7, 9, 11, and 13 are shown in FIGS. 2 to 8 and 15 to 28.

### Example 3

### Vector Construction for Soybean Transformation

Plant transformation binary vectors to over-express the genes described by SEQ ID NO:1, 3, 5, 7, 9, 135, 137, 139, 141, 143, 145, 147, 149, 151, 153, 155, 157, 159, and 161 were generated using constitutive and soybean cyst nematode (SCN) inducible promoters. For this, the open reading frames described by SEQ ID NO:1, 3, 5, 7, 9, 135, 137, 139, 141, 143, 145, 147, 149, 151, 153, 155, 157, 159, and 161 were operably linked to a constitutive ubiquitin promoter and the SCN inducible promoters TPP-like and MtN3-like. The resulting plant binary vectors contain a plant transformation selectable marker consisting of a modified Arabidopsis AHAS gene conferring tolerance to the herbicide Arsenal. The binary vectors designed to overexpress the proteins were transformed into disarmed A. rhizogenes strain K599 in preparation for transformation and SCN bioassay to determine effect on SCN cyst count.

### Example 4

### Nematode Bioassay

A bioassay to assess nematode resistance conferred by the polynucleotides described herein was performed using a rooted plant assay system disclosed in commonly owned copending U.S. Ser. No. 12/001,234. Transgenic roots are generated after transformation with the binary vectors described in Example 3. Multiple transgenic root lines are sub-cultured and inoculated with surface-decontaminated race 3 SCN second stage juveniles (J2) at the level of about 500 J2/well. Four weeks after nematode inoculation, the cyst number in each well is counted. For each transformation construct, the number of cysts per line is calculated to determine the average cyst count and standard error for the construct. The cyst count values for each transformation construct is compared to the cyst count values of an empty vector control tested in parallel to determine if the construct tested results in a reduction in cyst count. Bioassay results of constructs containing the genes described by SEQ ID NOs 3, 5, 139, 153, 157, and 159 resulted in a general trend of reduced soybean cyst nematode cyst count over many of the lines tested in at least one construct containing a constitutive or SCN inducible promoter operably linked to each of the genes described. Bioassay results of constructs containing the genes described by SEQ ID NOs 9, 147, and 149 resulted in a general trend of increased root mass over many of the lines tested in at least one construct containing a constitutive or SCN inducible promoter operably linked to each of the genes described. Bioassay results of constructs containing the genes described by SEQ ID NOs 1, 7, 135, 137, 141, 143, 145, 151, 155, 161 resulted in no observable effect on soybean cyst nematode cyst count or increased root mass.

Those skilled in the art will recognize, or will be able to ascertain using no more than routine experimentation, many equivalents to the specific embodiments of the invention described herein. Such equivalents are intended to be encompassed by the following claims.

1. An expression vector comprising a polynucleotide selected from the group consisting of:
a) a polynucleotide having a sequence as defined in SEQ ID NO: 1, 3, 5, 7, 9, 135, 137, 139, 141, 143, 145, 147, 149, 151, 153, 155, 157, 159, or 161;
b) a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162;
c) a polynucleotide having 70% sequence identity to a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant;
d) a polynucleotide encoding a polypeptide having 70% sequence identity to a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant;
e) a polynucleotide hybridizing under stringent conditions to a polynucleotide comprising a polynucleotide having a sequence as defined in SEQ ID NO: 1, 3, 5, 7, 9, 135, 137, 139, 141, 143, 145, 147, 149, 151, 153, 155, 157, 159, or 161, wherein said polynucleotide confers increased nematode resistance to a plant; and
f) a polynucleotide hybridizing under stringent conditions to a polynucleotide under stringent conditions to a polynucleotide comprising a polynucleotide encoding a polypeptide having a sequence as defined in SEQ ID NO: 2, 4, 6, 8, 10, 136, 138, 140, 142, 144, 146, 148, 150, 152, 154, 156, 158, 160, or 162, wherein said polynucleotide confers increased nematode resistance to a plant.

2. The expression vector of claim 1, further comprising one or more transcription regulatory elements operably linked to one or more polynucleotide(s) of claim 1.

3. The expression vector of claim 2, wherein the transcription regulatory element is (i) a promoter regulating constitutive expression of an operably linked polynucleotide in a plant, (ii) a promoter regulating tissue-specific expression of an operably linked polynucleotide in a plant or (iii) a promoter regulating expression of an operably linked polynucleotide in syncytia site of a plant upon nematode infection.

4. A plant comprising the expression vector of claim 1, 2, or 3.

5. The plant of claim 4, further described as a monocot.

6. The plant of claim 5, selected from the group consisting of maize, wheat, rice, barley, oat, rye, sorghum, Brachypodium sp., pearl millet, banana, and ryegrass.

7. The plant of claim 4, further described as a dicot.

8. The plant of claim 7, selected from the group consisting of pea, pigeonpea, Lotus, sp., Medicago truncatula, alfalfa, soybean, carrot, celery, tomato, potato, cotton, tobacco, pepper, oilseed rape, beet, cabbage, cauliflower, broccoli, lettuce, and Arabidopsis thaliana.

9. A seed generated from the plant of any one of claims 4 to 8, wherein the seed is true breeding for the polynucleotide of claim 1 or 2.

10. A method of producing a transgenic plant comprising a polynucleotide, wherein the method comprises the steps of:
a) introducing into a plant cell the expression vector of any one of claims 1 to 3; and
b) generating from the plant cell the transgenic plant expressing the polynucleotide.

11. A method of producing a transgenic plant comprising a polynucleotide, wherein expression of the polynucleotide in the plant results in the plant demonstrating increased resistance to nematodes as compared to wild type controls, and wherein the method comprises the steps of:
a) introducing into the plant the expression vector of any one of claims 1 to 3; and
b) selecting transgenic plants with increased pathogen resistance.

12. The method of claim 11, wherein the plant is a monocot.

13. The method of claim 12, wherein the plant is selected from the group consisting of maize, wheat, rice, barley, oat, rye, sorghum, Brachypodium sp., pearl millet, banana, and ryegrass.

14. The method of claim 11, wherein the plant is a dicot.

15. The method of claim 20, wherein the plant is selected from the group consisting of pea, pigeonpea, canola, Lotus, sp., Medicago truncatula, alfalfa, soybean, carrot, celery, tomato, potato, cotton, tobacco, pepper, oilseed rape, beet, cabbage, cauliflower, broccoli, lettuce, and Arabidopsis thaliana.

16. A method of increasing root growth in a crop plant, the method comprising the steps of transforming a crop plant cell with an expression vector comprising a polynucleotide selected from the group consisting of a polynucleotide having a sequence as defined in SEQ ID NO:9, 147, or 149 and a polynucleotide encoding a polypeptide having a sequence as defined in SEQ ID NO:10, 148, and 150; and selecting transgenic plants having increased root growth.


SEQUENCE LISTING (162 sequences)

SEQ ID NO: 1 - DNA, 351 bp, Escherichia coli
atggtaaagaaaagtgaatttgaacggggagacattgtgctggttggctttgatccagcaagcggccatgaacagcaaggtgctggtcgacctgcgcttgtgctctccgttcaagcctttaatcaactgggaatgacgctggtggcccccattacgcagggcggaaattttgcccgttatgccggatttagcgttcctttacattgcgaagaaggcgatgtgcacggcgtggtgctggtgaatcaggtgcggatgatggatctacacgcccggctggcaaagcgtattggtctggctgcggatgaggtggtggaagaggcgttattacgcttgcaggcggtggtggaataa

SEQ ID NO: 2 - protein, 116 aa, Escherichia coli
MVKKSEFERGDIVLVGFDPASGHEQQGAGRPALVLSVQAFNQLGMTLVAPITQGGNFARYAGFSVPLHCEEGDVHGVVLVNQVRMMDLHARLAKRIGLAADEVVEEALLRLQAVVE

SEQ ID NO: 3 - DNA, 816 bp, Saccharomyces cerevisiae
atgccttcgctaacccccagatgtatcattgtcagacacggtcaaactgaatggtccaagtcaggccagtatactggtttgacagatctaccgttaacgccctacggtgagggccaaatgttgaggaccggtgagagtgttttccgcaataatcagtttttgaatccagacaacatcacttatatcttcacctctccacgtttgcgtgccaggcaaactgtggatttggttttgaaaccattaagcgacgagcaaagagctaagatccgtgtggtggtagacgacgacttgcgagagtgggagtacggtgactacgagggaatgctgactcgagaaatcattgaattgagaaagtcacgcggtttggacaaggagaggccatggaatatctggagagatgggtgtgagaacggtgagactactcagcaaattgggttgagactttcccgcgctattgccagaatccagaacttgcaccgcaagcaccagagtgagggcagagcatcagacatcatggtctttgcgcacggacatgcattgcgttattttgctgctatttggtttggactgggtgtgcaaaagaagtgtgagacgattgaagaaattcaaaatgtcaaatcttatgatgacgacacagttccatatgtgaaattggaatcttacagacatttggtagacaatccatgtttcttactggacgccggtgggattggtgttttgtcatacgctcaccacaacattgacgaacctgcattggaattagcaggtccatttgtctcaccaccagaggaggaatcccagcatggcgatgtgtaa

SEQ ID NO: 4 - protein, 271 aa, Saccharomyces cerevisiae
MPSLTPRCIIVRHGQTEWSKSGQYTGLTDLPLTPYGEGQMLRTGESVFRNNQFLNPDNITYIFTSPRLRARQTVDLVLKPLSDEQRAKIRVVVDDDLREWEYGDYEGMLTREIIELRKSRGLDKERPWNIWRDGCENGETTQQIGLRLSRAIARIQNLHRKHQSEGRASDIMVFAHGHALRYFAAIWFGLGVQKKCETIEEIQNVKSYDDDTVPYVKLESYRHLVDNPCFLLDAGGIGVLSYAHHNIDEPALELAGPFVSPPEEESQHGDV

SEQ ID NO: 5 - DNA, 1290 bp, Escherichia coli
atggaaacgactcaaaccagcacgattgcgtcgaaagactctcgtagtgcctggcgcaagacagacaccatgtggatgctgggcctttacggcacggcaatcggcgcgggcgtgctgttcctgccaatcaacgccggtgttggcggtatgatcccgctgatcatcatggctatccttgcgttcccgatgacgttttttgctcaccgcggcctgactcgcttcgtactgtctggtaaaaacccgggcgaagacatcaccgaggttgtagaagaacactttggtattggcgcaggtaaactgattaccctgctctacttcttcgctatctacccgatcctgctggtttatagcgtggcaatcaccaataccgttgaaagcttcatgtctcaccagctgggtatgacgccaccgccgcgtgcgattctgtcgctgatcctgatcgtgggtatgatgaccatcgttcgcttcggtgagcagatgatcgttaaagcgatgagtattctggtattcccgtttgttggcgtactgatgctgctggctctgtacctgatcccgcagtggaacggcgctgcactggaaacgctgtctctggacactgcatctgcaaccggaaacggtctgtggatgaccctgtggctggcaattccggtaatggtgttctcgttcaaccactctccgatcatctcttctttcgccgttgcgaagcgtgaagagtacggcgatatggcagaacagaaatgctcgaagatcctggcattcgcacacatcatgatggtgctgaccgtaatgttcttcgtcttcagctgtgtactgagcctgactccggcagacctggctgcggctaaagagcagaacatctcgattctgtcttacctggctaaccactttaacgcaccggttatcgcgtggatggctccgattatcgcgattatcgctatcaccaaatccttcctcggtcactacctgggcgcacgtgaaggcttcaacggtatggtgattaaatctctgcgtggtaaaggtaagtctatcgaaatcaacaagctgaaccgtatcactgcgctgttcatgctggtaacgacctggattgttgccaccctgaacccgagcatcctgggtatgattgaaaccctgggcggtccaatcatcgcgatgatcctgttcctgatgccgatgtacgcaattcagaaagtaccggcaatgcgtaagtacagcggtcacatcagcaacgtattcgttgtcgtgatgggtctgattgcaatctccgcaatcttctactctctgttcagctaa

SEQ ID NO: 6 - protein, 429 aa, Escherichia coli
METTQTSTIASKDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFMSHQLGMTPPPRAILSLILIVGMMTIVRFGEQMIVKAMSILVFPFVGVLMLLALYLIPQWNGAALETLSLDTASATGNGLWMTLWLAIPVMVFSFNHSPIISSFAVAKREEYGDMAEQKCSKILAFAHIMMVLTVMFFVFSCVLSLTPADLAAAKEQNISILSYLANHFNAPVIAWMAPIIAIIAITKSFLGHYLGAREGFNGMVIKSLRGKGKSIEINKLNRITALFMLVTTWIVATLNPSILGMIETLGGPIIAMILFLMPMYAIQKVPAMRKYSGHISNVFVVVMGLIAISAIFYSLFS

SEQ ID NO: 7 - DNA, 1425 bp, Escherichia coli
atgaaaaaaaccacattagcactgagtgcactggctctgagtttaggtttggcgttatctccgctctctgcaacggcggctgagacttcttcagcaacgacagcccagcagatgccaagccttgcaccgatgctcgaaaaggtgatgccttcagtggtcagcattaacgtagaaggtagcacaaccgttaatacgccgcgtatgccgcgtaatttccagcagttcttcggtgatgattctccgttctgccaggaaggttctccgttccagagctctccgttctgccagggtggccagggcggtaatggtggcggccagcaacagaaattcatggcgctgggttccggcgtcatcattgatgccgataaaggctatgtcgtcaccaacaaccacgttgttgataacgcgacggtcattaaagttcaactgagcgatggccgtaagttcgacgcgaagatggttggcaaagatccgcgctctgatatcgcgctgatccaaatccagaacccgaaaaacctgaccgcaattaagatggcggattctgatgcactgcgcgtgggtgattacaccgtagcgattggtaacccgtttggtctgggcgagacggtaacttccgggattgtctctgcgctggggcgtagcggcctgaatgccgaaaactacgaaaacttcatccagaccgatgcagcgatcaaccgtggtaactccggtggtgcgctggttaacctgaacggcgaactgatcggtatcaacaccgcgatcctcgcaccggacggcggcaacatcggtatcggttttgctatcccgagtaacatggtgaaaaacctgacctcgcagatggtggaatacggccaggtgaaacgcggtgagctgggtattatggggactgagctgaactccgaactggcgaaagcgatgaaagttgacgcccagcgcggtgctttcgtaagccaggttctgcctaattcctccgctgcaaaagcgggcattaaagcgggtgatgtgatcacctcactgaacggtaagccgatcagcagctttgccgcactgcgtgctcaggtgggtactatgccggtaggcagcaaactgaccctgggcttactgcgcgacggtaagcaggttaacgtgaacctggaactgcagcagagcagccagaatcaggttgattccagctccatcttcaacggcattgaaggcgctgagatgagcaacaaaggcaaagatcagggcgtggtagtgaacaacgtgaaaacgggcactccggctgcgcagatcggcctgaagaaaggtgatgtgattattggcgcgaaccagcaggcagtgaaaaacatcgctgaactgcgtaaagttctcgacagcaaaccgtctgtgctggcactcaacattcagcgcggcgacagcaccatctacctgttaatgcagtaa

SEQ ID NO: 8 - protein, 474 aa, Escherichia coli
MKKTTLALSALALSLGLALSPLSATAAETSSATTAQQMPSLAPMLEKVMPSVVSINVEGSTTVNTPRMPRNFQQFFGDDSPFCQEGSPFQSSPFCQGGQGGNGGGQQQKFMALGSGVIIDADKGYVVTNNHVVDNATVIKVQLSDGRKFDAKMVGKDPRSDIALIQIQNPKNLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVKNLTSQMVEYGQVKRGELGIMGTELNSELAKAMKVDAQRGAFVSQVLPNSSAAKAGIKAGDVITSLNGKPISSFAALRAQVGTMPVGSKLTLGLLRDGKQVNVNLELQQSSQNQVDSSSIFNGIEGAEMSNKGKDQGVVVNNVKTGTPAAQIGLKKGDVIIGANQQAVKNIAELRKVLDSKPSVLALNIQRGDSTIYLLMQ

SEQ ID NO: 9 - DNA, 1479 bp, Saccharomyces cerevisiae
atgtcaaaggcagtaggtgatttaggcttagttggtttagccgtgatgggtcaaaatttgatcttaaacgcagcggatcacggatttaccgtggttgcttataataggacgcaatcaaaggtagataggtttctagctaatgaggcaaaaggaaaatcaataattggtgcaacttcaattgaggacttggttgcgaaactaaagaaacctagaaagattatgcttttaatcaaagccggtgctccggtcgacactttaataaaggaacttgtaccacatcttgataaaggcgacattattatcgacggtggtaactcacatttcccggacactaacagacgctacgaagagctaacaaagcaaggaattctttttgtgggctctggtgtctcaggcggtgaagatggtgcacgttttggtccatctttaatgcctggtgggtcagcagaagcatggccgcacatcaagaacatctttcaatctattgccgccaaatcaaacggtgagccatgctgcgaatgggtggggcctgccggttctggtcactatgtgaagatggtacacaacggtatcgagtacggtgatatgcagttgatttgcgaggcttacgatatcatgaaacgaattggccggtttacggataaagagatcagtgaagtatttgacaagtggaacactggagttttggattctttcttgattgaaatcacgagggacattttaaaattcgatgacgtcgacggtaagccattggtggaaaaaattatggatactgccggtcaaaagggtactggtaaatggactgcaatcaacgccttggatttaggaatgccagtcactttaattggggaggctgttttcgctcgttgtttgtcagccataaaggacgaacgtaaaagagcttcgaaacttctggcaggaccaacagtaccaaaggatgcaatacatgatagagaacaatttgtgtatgatttggaacaagcattatacgcttcaaagattatttcatatgctcaaggtttcatgctgatccgcgaagctgccagatcatacggctggaaattaaacaacccagctattgctctaatgtggagaggtggctgtataatcagatctgtgttcttagctgagattacgaaggcttatagggacgatccagatttggaaaatttattattcaacgagttcttcgcttctgcagttactaaggcccaatccggttggagaagaactattgcccttgctgctacttacggtattccaactccagctttctctactgctttagcgttttacgacggctatagatctgagaggctaccagcaaacttgttacaagcgcaacgtgattattttggcgctcatacatttagaattttacctgaatgtgcttctgcccatttgccagtagacaaggatattcatatcaattggactgggcacggaggtaatatatcttcctcaacctaccaagcttaa

SEQ ID NO: 10 - protein, 492 aa, Saccharomyces cerevisiae
MSKAVGDLGLVGLAVMGQNLILNAADHGFTVVAYNRTQSKVDRFLANEAKGKSIIGATSIEDLVAKLKKPRKIMLLIKAGAPVDTLIKELVPHLDKGDIIIDGGNSHFPDTNRRYEELTKQGILFVGSGVSGGEDGARFGPSLMPGGSAEAWPHIKNIFQSIAAKSNGEPCCEWVGPAGSGHYVKMVHNGIEYGDMQLICEAYDIMKRIGRFTDKEISEVFDKWNTGVLDSFLIEITRDILKFDDVDGKPLVEKIMDTAGQKGTGKWTAINALDLGMPVTLIGEAVFARCLSAIKDERKRASKLLAGPTVPKDAIHDREQFVYDLEQALYASKIISYAQGFMLIREAARSYGWKLNNPAIALMWRGGCIIRSVFLAEITKAYRDDPDLENLLFNEFFASAVTKAQSGWRRTIALAATYGIPTPAFSTALAFYDGYRSERLPANLLQAQRDYFGAHTFRILPECASAHLPVDKDIHINWTGHGGNISSSTYQA

SEQ ID NO: 11 - DNA, 351 bp, Escherichia coli
atggtaaagaaaagtgaatttgaacggggagacattgtgctggttggctttgatccagcaagcggccatgaacagcaaggtgctggtcgacctgcgcttgtgctctccgttcaagcctttaatcaactgggaatgacgctggtggcccccattacgcagggtggaaattttgcccgttatgccggatttagcgttcctttacattgcgaagaaggcgatgtgcacggcgtggtgctggttaatcaggtgcggatgatggatctacgcgcccggctggcaaagcgtattggtctggctgcggatgaggtggtggaagaggcgttattacgcttgcaggcggtggtggaataa

SEQ ID NO: 12 - protein, 116 aa, Escherichia coli
MVKKSEFERGDIVLVGFDPASGHEQQGAGRPALVLSVQAFNQLGMTLVAPITQGGNFARYAGFSVPLHCEEGDVHGVVLVNQVRMMDLRARLAKRIGLAADEVVEEALLRLQAVVE

SEQ ID NO: 13 - DNA, 351 bp, Burkholderia vietnamiensis
atggtgaggcgggtcaagtttgaccggggcgacattgtgcgcgtgagcctgaacccgaccatcggccgcgagcagcagggcgatttccgtcctgcgctcgtgctgtctcccgccgcattcaatgcgttgggggtggcgctcgttgcgccgatcacacagggtggggagtgtgcgcgcttcgcaggattcgccgtgccgctctcaggctcgggcaccgagacgcagggcgtggccttggtgaacatggtccggatgctcgatctggacgcgcggggtgcacgcaagatcgagcgggctccggccgaggtggtcgaagatgcgctggctcggctgcaaacgataatcgagtag

SEQ ID NO: 14 - protein, 116 aa, Burkholderia vietnamiensis
MVRRVKFDRGDIVRVSLNPTIGREQQGDFRPALVLSPAAFNALGVALVAPITQGGECARFAGFAVPLSGSGTETQGVALVNMVRMLDLDARGARKIERAPAEVVEDALARLQTIIE

SEQ ID NO: 15 - DNA, 348 bp, Pseudomonas putida
gtgaaacggttgaaattcgccaggggtgatattgttcgcgtcaacctggacccaacagtcgggcgggaacagcagggctccggccgacctgcactggtacttactccggctgcgttcaatgcttcaggcctggctgtaatcatcccgatcactcaaggtggggatttcgcgaggcatgcgggtttcgctgtcacgctcagcggtgcgggcacgcagactcagggggtgatgctttgcaaccaggtgcgcacagtcgaccttgaagcacgatttgccaagcgcatagagtcggtgcctgaagctgtcatcctggatgcactggcgcgtgtgcaaaccctattcgattaa

SEQ ID NO: 16 - protein, 115 aa, Pseudomonas putida
MKRLKFARGDIVRVNLDPTVGREQQGSGRPALVLTPAAFNASGLAVIIPITQGGDFARHAGFAVTLSGAGTQTQGVMLCNQVRTVDLEARFAKRIESVPEAVILDALARVQTLFD

SEQ ID NO: 17 - DNA, 348 bp, Pseudomonas syringae
ttgaaacgggttaaattcaaccggtcggatattgtgcgactgaacctgaatccaaccgcaggccgtgaacaacagggcgattttcgtccagcgttgattctgacgcctgcggcttataacgcatcgggccttgtaattattgtacccatcacgcagggcggcgacttcgctcggtatgccggtttcgcggtgcctttaagcggttcaggtaccgaaacgcagggtgtcgttctttgcaaccagattcgaaccgttgaccttgaggctcgtggcgcaaagcgtgttgagtcagttcccgagatgataatcgacgatgtgctggctcgcgttcaagccctttttgaatag

SEQ ID NO: 18 - protein, 115 aa, Pseudomonas syringae
MKRVKFNRSDIVRLNLNPTAGREQQGDFRPALILTPAAYNASGLVIIVPITQGGDFARYAGFAVPLSGSGTETQGVVLCNQIRTVDLEARGAKRVESVPEMIIDDVLARVQALFE

SEQ ID NO: 19 - DNA, 336 bp, Mycobacterium celatum
ctgacggaacgcggcgacatctacatcgtttcgcttgacccgacgtcgggacatgagcagagcggcacgcgcccagtattggtcgtgtccccgggcgcgtttaatcgcctgacgaaaacaccggtcgtgctacctataacacgcggcgggaactttgcccgaacggcagggttcgctgtctcgctgaccgatgcgggtactcgcaccgccggcgtaatacgctgcgatcagcctcgctcgattgatatccgcgcccgtaaaggccgcaaggttgaacgtgtgccgtctggggttcttgacgaagcgttggccaagctcgccacgatcttgacttga

SEQ ID NO: 20 - protein, 111 aa, Mycobacterium celatum
MTERGDIYIVSLDPTSGHEQSGTRPVLVVSPGAFNRLTKTPVVLPITRGGNFARTAGFAVSLTDAGTRTAGVIRCDQPRSIDIRARKGRKVERVPSGVLDEALAKLATILT

SEQ ID NO: 21 - DNA, 339 bp, Synechococcus sp.
gtggtgagagcgccggagcgcggcgatctgatctggctcagcttcacgccacagagtggacgtgagcaggctggacgccgaccggcagtggtggtgtcgccatcggcctacaacagcaaggttggcctggctttggtgtgtccaatcaccagcaaggtgaagggatacccgtttgaagtggcactgccggaacaaggaacggtgcagggtgtgatcttggcggatcaactacgcagcctggactggcgcagccaagaagccgagctgatcgccaaggctccgattcctgtggtggaacgcgttctgcagctggttggcgcgctgttgagcagtccgtga

SEQ ID NO: 22 - protein, 112 aa, Synechococcus sp.
MVRAPERGDLIWLSFTPQSGREQAGRRPAVVVSPSAYNSKVGLALVCPITSKVKGYPFEVALPEQGTVQGVILADQLRSLDWRSQEAELIAKAPIPVVERVLQLVGALLSSP

SEQ ID NO: 23 - DNA, 402 bp, Erwinia amylovora
atgctgaaatctcagctgaagaacgagaatggctggatgcgccggcgactggtcaggaggaaatctgacatggaaagaggggaaatctggcttgtctcgcttgatcctagcgcaggtcatgagcagcagggaacgcggccggtactgattgtcacgccggctgcctttaaccgcgtgacccgcctgcctgttgttgtgcccgtgaccagcggaggcaattttgcccgcactgctggctttgcggtgtcgcttgacggcgccggcatacgtaccaccggcgttgtgcgttgcgatcaaccccggacgatcgatatgaaagcccgcggcggcaaacgacttgaacgggtgcctgagactatcatggatgacgttcttggtcgtctggccacgattctgacttga

SEQ ID NO: 24 - protein, 133 aa, Erwinia amylovora
MLKSQLKNENGWMRRRLVRRKSDMERGEIWLVSLDPSAGHEQQGTRPVLIVTPAAFNRVTRLPVVVPVTSGGNFARTAGFAVSLDGAGIRTTGVVRCDQPRTIDMKARGGKRLERVPETIMDDVLGRLATILT

SEQ ID NO: 25 - DNA, 360 bp, Agrobacterium tumefaciens
atggtccgcaaccagatccccaagcgcggcgacgtttatctggttgatctaaaccctgtcgtaggcagcgaaatcaaggacgaacatcgctgtgtcgtcatcacgcccagagaaattaacgcggtcggactctgtctcgtcgtcccggtgaccaccggcggcatgtttacgcgcaaggcagggcttgccgtaaatatatccggccacaagacaacgggcgtcgctttgtgcaatcaggtgagaagcatggatatcgtcgcccgggttgcccagaagaaagcgaaatatatcgaaaccctcgatgatgcgacgatcgatgaaatcgccgggcgcgtcatcagcatgatcgatccagcttga

SEQ ID NO: 26 - protein, 119 aa, Agrobacterium tumefaciens
MVRNQIPKRGDVYLVDLNPVVGSEIKDEHRCVVITPREINAVGLCLVVPVTTGGMFTRKAGLAVNISGHKTTGVALCNQVRSMDIVARVAQKKAKYIETLDDATIDEIAGRVISMIDPA

SEQ ID NO: 27 - DNA, 348 bp, Neisseria meningitidis
atgtatattcccgacaaaggcgatattttccatttgaatttcgacccttccagcggcaaggaaatcaagggcgggcggtttgcgctggctctgtctccaaaagcattcaaccgcgcaacgggattggtttttgcctgccccatttcacaggggaatgcagcggctgcacgaagcagcggcatgatttcaaccttactcggtgcaggaacggaaacgcagggcaatgtccactgccaccagctcaaatctctggactggcaaatccgcaaggcttcttttaaagaaactgtacccgattatgtattggacgatgtgctggcgcgcatcggcgccgtcttattcgattaa

SEQ ID NO: 28 - protein, 115 aa, Neisseria meningitidis
MYIPDKGDIFHLNFDPSSGKEIKGGRFALALSPKAFNRATGLVFACPISQGNAAAARSSGMISTLLGAGTETQGNVHCHQLKSLDWQIRKASFKETVPDYVLDDVLARIGAVLFD

SEQ ID NO: 29 - DNA, 360 bp, Erwinia carotovora
ttgatggctcggatgccaaaaagaggtgaaatttggttagtcaatccagatccaattgctggtagagagattgctgggccgcattacttcctcgtaatatcggttgataccgtgaacaaagaaaccggcgttacagcctgtgccgcgataacatcaggcgcagggtccttgcgcgaaaaaaacatcgtcgtttatatcggcggtggtgatacagagaaaggcaaagtgactggagtgattctttgccaccaactgaactctcttgattttcaggcaagaagcgcgaaatatattgatacggtgtcacctcaggtcatggctgatgtcgaaatcacactggcgaacatattgggcatatga

SEQ ID NO: 30 - protein, 119 aa, Erwinia carotovora
MMARMPKRGEIWLVNPDPIAGREIAGPHYFLVISVDTVNKETGVTACAAITSGAGSLREKNIVVYIGGGDTEKGKVTGVILCHQLNSLDFQARSAKYIDTVSPQVMADVEITLANILGI

SEQ ID NO: 31 - DNA, 363 bp, Synechocystis sp.
atggttatgacctcaaccgtcccgcaatatcctgaacggggggcggtaattcgcttaaacctcaatcccacccaggggcgagaacaaatgggagaggcgcggccatgtctggttttgagccatactgcctttaataaggcccgcaatggcttgatcattgtttccccaattacgaataccattaagccagaaattcagacgttggttgtccttcctgatggttatcgggtacaaggatcggttattgctgagcaaatccgcactgtggatttaagtttgcgttggtggcgggatacgggagaagtccttccccctagttttgtggatcaggtcttggcggttctccagctaattattggatga

SEQ ID NO: 32 - protein, 120 aa, Synechocystis sp.
MVMTSTVPQYPERGAVIRLNLNPTQGREQMGEARPCLVLSHTAFNKARNGLIIVSPITNTIKPEIQTLVVLPDGYRVQGSVIAEQIRTVDLSLRWWRDTGEVLPPSFVDQVLAVLQLIIG

SEQ ID NO: 33 - DNA, 348 bp, Crocosphaera watsonii
atgaagattgaaagaggtcaaatatattttgttaatcttaaccccattcaaggtaaagaacaagcaggaaatcgtcctgttttagtattatccattaattctatcaatgacttacctttagttgtcacagtggttgtaggtactaagggtaaaaatattcgccaagattatccgacaaatgttagggtttctcctgatgaaagtggactttcgttagaaacaatatttctctgttttcaaattcgttcaattgataaaaatcgtttttcttctgcaccagttggtaaattgtcagaggaaaaaatgcaagaaattgaaacagctattcgttattgtttaggtttatag

SEQ ID NO: 34 - protein, 115 aa, Crocosphaera watsonii
MKIERGQIYFVNLNPIQGKEQAGNRPVLVLSINSINDLPLVVTVVVGTKGKNIRQDYPTNVRVSPDESGLSLETIFLCFQIRSIDKNRFSSAPVGKLSEEKMQEIETAIRYCLGL

SEQ ID NO: 35 - DNA, 840 bp, Candida glabrata
atgggtcacagtccaactcctagatgtattattgtcagacatggccagaccgagtggtctaagtccggtcagtacaccggtttgactgacttgccattgactgactatggtgttggccaaatgctgaggaccggtgagtctatcttctccaacaaccgtttcatcaaccctgaccacatcacatatatcttcacttctccaagaaccagggccaagcagaccattgagttggttttgaagccattgagcgaggagcaaaggtctaagatcaaggttattgttgacaatgacttgagagaatgggagtacggtgactatgaagggctattgaccaaggagattgtcgagctgcgtaagtcccgtggtttggacaaagctcgcccatggaatatctggagagatggctgtgagaacggtgaaactactgaagaagtcggtttgagattgtctagagtcatcgcacgtatccaaaacctgcacaagaagcaccaggaacaaggcataccatctgatatcatggtgtttgctcatggacatgctttgcgttactttgcggccatctggctacgtctaggtgaagagaaagagtgtatcactgctttctctaagcagaaggttaagtcctatgaggaggatgagtctgtggtcccatacgttgagattaccaagttcagacacttggtggacaacccattcttcttacttgacgctggtggtatcggtgtattgtcttatgcccaccacagcattgatgaacccgccttggacctagctggtgcgttcatttccccaccagaggaagagtcccaacacgctccagttccaaacaagaattactga

SEQ ID NO: 36 - protein, 279 aa, Candida glabrata
MGHSPTPRCIIVRHGQTEWSKSGQYTGLTDLPLTDYGVGQMLRTGESIFSNNRFINPDHITYIFTSPRTRAKQTIELVLKPLSEEQRSKIKVIVDNDLREWEYGDYEGLLTKEIVELRKSRGLDKARPWNIWRDGCENGETTEEVGLRLSRVIARIQNLHKKHQEQGIPSDIMVFAHGHALRYFAAIWLRLGEEKECITAFSKQKVKSYEEDESVVPYVEITKFRHLVDNPFFLLDAGGIGVLSYAHHSIDEPALDLAGAFISPPEEESQHAPVPNKNY

SEQ ID NO: 37 - DNA, 816 bp, Kluyveromyces lactis
atgtctaaagttactccccgttgtattatcgtccgtcatggacaaactgaatggtcaaaatccggtcaatataccggattaactgatattccgttgactccatatggtgttgaacagatgagaagaactggtaaatgcgtctttggtaacaagttcattgacccaaaccatatcacatacattttcacttctcctcgtcaacgtgctagaaagactgttgatttaattttggaatcccttactgaagaacaacgttcaaagatcagaatcgtagtcgataacgatctaagagaatgggaatatggtgattacgagggtctgttgacccatgaaattgtcaaattgagaaaatcccgtggtttggatcaagaaaggccttggaacatctggcgtgatggttgtgaaaacggtgaaagcaccaagcaaatcggttgtagactttcccgtgtcatttccagaattcaaaacctacatcgccaacaacagaaagacggagtaccaagtgatatcttggtgtttgcacatggccatgcattacgttatttcgcatccttgtggatcaaacatggattagaagaaaaatatgaaccatcattcgatccaccaaaacctacttataacgatgagactgttcctaatgtaacaatggaaaagtacagatacttggttgataatccaaacttcttgttggatgctggtggtatgggagttctttcttatgcacaccataatattgacgaacctgctctagcgttggccggaacatttattgccccaccagaggaagaatctcaacatgaaccggtagttgaataa

SEQ ID NO: 38 - protein, 271 aa, Kluyveromyces lactis
MSKVTPRCIIVRHGQTEWSKSGQYTGLTDIPLTPYGVEQMRRTGKCVFGNKFIDPNHITYIFTSPRQRARKTVDLILESLTEEQRSKIRIVVDNDLREWEYGDYEGLLTHEIVKLRKSRGLDQERPWNIWRDGCENGESTKQIGCRLSRVISRIQNLHRQQQKDGVPSDILVFAHGHALRYFASLWIKHGLEEKYEPSFDPPKPTYNDETVPNVTMEKYRYLVDNPNFLLDAGGMGVLSYAHHNIDEPALALAGTFIAPPEEESQHEPVVE

SEQ ID NO: 39 - DNA, 741 bp, Debaryomyces hansenii
atgagcccatctaaagcaccaactccaagagttatatttattcgccacggccaaaccgaatggtcaaaatctggccaatatacgtccattactgatttaccattaacagatttcggagtcaaacagatgcggaatactggtaaacacttaattgggcctagtccattccaattgatcaaaccggaaaacttgaagaatgtctttacatctccaagattgcgtgccaagcaaacggttgagttgttgctcgaagggttgaacgatatggcaaaggccaagttgcttaccaccgaggaagataacttaagagagtgggaatacggtgattacgaaggattattgacgtcccagattattgaattaagaaacaagagaggtttggataaggatttacctaaaggtgaagaatggaacatttggagggacgggtgtgaaaacggtgaaacgcacgagcaagtttccgcaagattggacaaggttatcacgaacatcagagctatccacgaaaaggcgattcaagataatgttccttgcgatgtcatagttgttggacatggtcatatcttgagatgttttgctgctagatgggtgggtaaagacattaatgtcaatccgcaagtcgtgttggatgctggtggagttggagttttgagctatcaacaccataatattcacgagcctgctttatgcttagctggagcatttgttgtcccagttgaagaggaagggtctgatatataa

SEQ ID NO: 40 - protein, 246 aa, Debaryomyces hansenii
MSPSKAPTPRVIFIRHGQTEWSKSGQYTSITDLPLTDFGVKQMRNTGKHLIGPSPFQLIKPENLKNVFTSPRLRAKQTVELLLEGLNDMAKAKLLTTEEDNLREWEYGDYEGLLTSQIIELRNKRGLDKDLPKGEEWNIWRDGCENGETHEQVSARLDKVITNIRAIHEKAIQDNVPCDVIVVGHGHILRCFAARWVGKDINVNPQVVLDAGGVGVLSYQHHNIHEPALCLAGAFVVPVEEEGSDI

SEQ ID NO: 41 - DNA, 723 bp, Candida albicans
atgacaaaaagtccttgtccaagattaatttttgtccgtcatggtcaaactgaatggtccaagagtggtcaatatacttcaagaaccgatctcgacttgactccatttggtgtcaaacaaatgagaaatacaggtaaaggtcttattggtcctggtaatttacaaatgatcaaaccggaaaatttaactcatatttttgtatctccaaggaagagagcacaacgtacctcacaattgctattagaagaagttgatccagaaattaaggataaaattccaattgaaatagatgaagatgtgagagaatgggagtatggtgaatacgaaggtttaaaaacaaacgaatttatcgaattgagaaagcaaaaaggattagataaggatctggagtggactatttggggtaaagggcgtgaaggaggtgaacaacattatgaagttgcagcaagattggatcgttttattgagaagatccaaaaaattcaccgtgaagctcttgctaaaggagtggcatctgatataattgttgttgcacatggtcatatattaagatgtcttgttgcaagatgggttaaacgtgagttaagtaccaatccccaattaattttggatgctggtggtgttggtgttttgagctaccaacatcacaatgtcgacgaaccagcaatttatttggctggtgcgtttactgtccctgttgaagaagaaggtgcagatatttaa

SEQ ID NO: 42 - protein, 240 aa, Candida albicans
MTKSPCPRLIFVRHGQTEWSKSGQYTSRTDLDLTPFGVKQMRNTGKGLIGPGNLQMIKPENLTHIFVSPRKRAQRTSQLLLEEVDPEIKDKIPIEIDEDVREWEYGEYEGLKTNEFIELRKQKGLDKDSEWTIWGKGREGGEQHYEVAARLDRFIEKIQKIHREALAKGVASDIIVVAHGHILRCLVARWVKRELSTNPQLILDAGGVGVLSYQHHNVDEPAIYLAGAFTVPVEEEGADI

SEQ ID NO: 43 - DNA, 726 bp, Candida albicans
atgtccaaaattccttatccaagattgatttttgttcgccacggtcaaactgaatggtctaaaagtggtcaacacacttcaactaccgacattgacttgactccatttggggtcgaacaaatgagaaatactggtagagcacttatcggtcctagtaatttacaaatgatcaaaccagagaatttgactcgtatttttgtttcaccaaggcaaagagcacaacaaactttacaattattattggaagatgttgaccccgaatttaaggacaaaatcccagttgaaatagatgaggatgtgagagaatgggattatggtgattacgaaggaataacgagtgctgaaatcaacgaattaaggaaaaagaaaggattagatgataaagatcacaaatggtccatatggagtgatggatgtgaaggaggtgaacaacattatgatgtagcaaagaggttggatagatttattgaaaaagtgagagagtttcatcgtcaagcaattgctaaaagggaaccatgtgatatcctcgtcgttgctcatggtcatatattgagatgtctaggagctagatgggtccaacgtgaattgaatgtcaatccacaattgattttggatgctggtggtgtaggaactttgagttatgaacaccataatatcgatgaaccatcaatctttttatcaggggcattcaccgtaccggttgccgaacaatgtgctgatctttag

SEQ ID NO: 44 - protein, 241 aa, Candida albicans
MSKIPYPRLIFVRHGQTEWSKSGQHTSTTDIDLTPFGVEQMRNTGRALIGPSNLQMIKPENLTRIFVSPRQRAQQTLQLLLEDVDPEFKDKIPVEIDEDVREWDYGDYEGITSAEINELRKKKGLDDKDHKWSIWSDGCEGGEQHYDVAKRLDRFIEKVREFHRQAIAKREPCDILVVAHGHILRCLGARWVQRELNVNPQLILDAGGVGTLSYEHHNIDEPSIFLSGAFTVPVAEQCADL

SEQ ID NO: 45 - DNA, 726 bp, Candida albicans
atgtccaaaattccttatccaagattgatttttgttcgccacggtcaaactgaatggtctaaaagtggtcaacatacttcaactaccgacattgacttgactccatttggggtcgaacaaatgagaaatactggtagagcacttatcggtcctagtaatttacaaatgatcaaaccagagaatttgactcgtatttttgtttcaccaaggcaaagagcacaacaaactttacaattattattggaagatgttgaccccgaatttaaggacaaaatcccagttgaaatagatgaggatgtgagagaatgggattatggtgattacgaaggaataacgagtgctgaaatcaacgaattaagaaaaaagaaaggattagatgataaagatcacaaatggtccatatggagtgatggatgtgaaggaggtgagcaacattatgatgtagcaaagaggttggatagatttattgaaaaagtaagagagtttcatcgtcaagcaattgctaaaaaggaaccatgtgatatcctcgtcgttgctcatggtcatatattgagatgtctaggagctagatgggtccaacgtgaattgaatgtcaatccacaattgattttggatgctggtggtgtaggaactttgagttatgaacaccataatatcgatgaaccatcaatctttttatcaggggcattcaccgtaccggttgccgaacaatgtgctgatctttag

SEQ ID NO: 46 - protein, 241 aa, Candida albicans
MSKIPYPRLIFVRHGQTEWSKSGQHTSTTDIDLTPFGVEQMRNTGRALIGPSNLQMIKPENLTRIFVSPRQRAQQTLQLLLEDVDPEFKDKIPVEIDEDVREWDYGDYEGITSAEINELRKKKGLDDKDHKWSIWSDGCEGGEQHYDVAKRLDRFIEKVREFHRQAIAKKEPCDILVVAHGHILRCLGARWVQRELNVNPQLILDAGGVGTLSYEHHNIDEPSIFLSGAFTVPVAEQCADL

SEQ ID NO: 47 - DNA, 741 bp, Yarrowia lipolytica
atggcccctcgagttatctttgtacgacacggcgagaccgaatggtcaaagtccggccaacacacgtcggtgactgatctgccattgactgagaacggagtcaagcgagtgcgagcgacgggacgggcgctggtgggccgaaaccggctggtgaacccggcgtacgtggagcacatttttgtttcgccccgatctcgtgcccagcagacgctcaagctcttttttgaggacgagcccgaggctctcgccaagatcccccagaccgtgaccgaagacattcgagagtgggactacggcaagtacgagggccgaaagtcagccgaaatccgggccgaccgaaccgcgcgaggcatcgacaaggacggccacaagtggaacatttggtccgacggctgcgaggacggagagtcgccccaacaggtgcagaagcgagtggacgagctcatcaaggagatccgggtgatccacaagaaggcgctcgacgagggcaaggagcattgcgacgtcatggtgttcgcacacggccacatcctgcgagtctttgctctgcgatgggtcaacggagacatcaccatcaacccggctctgattctcgaggcaggaggagtcggtgtgctgtcttacgagcataacaacattgaggagccagccatttacctgggaggagccttctttgtgcccgacgaggatgtggagaagaacagcggagtcattgcgctggctgggggagagcagaactag

SEQ ID NO: 48 - protein, 246 aa, Yarrowia lipolytica
MAPRVIFVRHGETEWSKSGQHTSVTDLPLTENGVKRVRATGRALVGRNRLVNPAYVEHIFVSPRSRAQQTLKLFFEDEPEALAKIPQTVTEDIREWDYGKYEGRKSAEIRADRTARGIDKDGHKWNIWSDGCEDGESPQQVQKRVDELIKEIRVIHKKALDEGKEHCDVMVFAHGHILRVFALRWVNGDITINPALILEAGGVGVLSYEHNNIEEPAIYLGGAFFVPDEDVEKNSGVIALAGGEQN

SEQ ID NO: 49 - DNA, 795 bp, Magnaporthe grisea
atgcgacaagcgtcaccaaagtcggggatgtcaacgcctcgtgtcttcattgtccgccacggcgagaccgagtggtccctcgacggccgccacactggctccacggatattcccctgacggccaatggcgagaagcgcgtgagggctaccggccgggccatggtcggaaatgaccgcctgatcgtgcccagaaagctcgcacacatatacgtatctcctcgtaagcgcgcccagcgcacctttgagctgctcaaccttggcctcaaggaccctctcccatgggaacctcatggcgacctggagaaggatcctcgtgactgcgacacggccaagatcgaggtcaccgaggacatccgcgagtgggactatggtgagtacgagggcatcaccagcccagagatcagggagatccgtaagaagcagggcatagaggggaggtgggatatctggagggatggatgtcctggtggagaaagccctgaggacataaccgcccggcttgatcgcctcatagccgacatccgtaacaagttccacgcgccggtgatggaaaagggagcaacggaccccaaggagccccagccagccagcgacgtgctcgtcgtggcgcatggtcatatcctccgggctttcgccatgcgctgggccggcaagagtctccaggatgggccgactttcatcctggaggctggcggtgtaggcagtcttagctatgaacaccacaacgtagccgagcctgccatcctgcttggcggtgcctttgtgattgacgttcccgaagcgtccaaggactga

SEQ ID NO: 50 - protein, 264 aa, Magnaporthe grisea
MRQASPKSGMSTPRVFIVRHGETEWSLDGRHTGSTDIPLTANGEKRVRATGRAMVGNDRLIVPRKLAHIYVSPRKRAQRTFELLNLGLKDPLPWEPHGDLEKDPRDCDTAKIEVTEDIREWDYGEYEGITSPEIREIRKKQGIEGRWDIWRDGCPGGESPEDITARLDRLIADIRNKFHAPVMEKGATDPKEPQPASDVLVVAHGHILRAFAMRWAGKSLQDGPTFILEAGGVGSLSYEHHNVAEPAILLGGAFVIDVPEASKD

SEQ ID NO: 51 - DNA, 720 bp, Aspergillus nidulans
atggccccccgctgcttcataatccgccacggcgaaactgaatggtcgctgaacgggcggcacacgggaatcacagacctgccactcaccgagaacggcgagaagcggattaaagcaactggaaaggctcttgtcggcaacgacaggttaattgcgccgaaaaagctagtccatgtatatgtctctcctcgcacccgcgcgcagcggacgctggaactgcttgaaatcgggtgtcgagagcgccttccctggacagagaagcgcaaggccgaatctgacgagccgatccggacggaagcgaaagtggagattacagaggctatccgagaatgggattacggcgactacgagggtttgacgagtaagcagatccgagagcgaagggcggaacaaggggaggggagctgggatatttggcgggatgggtgtcccgggggagaatcacctgaagacgtcatgaaacgactagacgcgctgattgctgagattcgagaaaagcaccacaagccgtgtttcgagggtaataaggagagcggcgacgtgcttatcgttgcgcacgggcacatcctccgtgcgtttgcgatgcgctggacgggccgaccgttaacggagacggcgctgattctggaggctggtggtgtaggcacattgagttacgagcaccataacatcgaagagccggcgatcatcctcggtggaggatttgtcgtggagaattga

SEQ ID NO: 52 - protein, 239 aa, Aspergillus nidulans
MAPRCFIIRHGETEWSLNGRHTGITDLPLTENGEKRIKATGKALVGNDRLIAPKKLVHVYVSPRTRAQRTLELLEIGCRERLPWTEKRKAESDEPIRTEAKVEITEAIREWDYGDYEGLTSKQIRERRAEQGEGSWDIWRDGCPGGESPEDVMKRLDALIAEIREKHHKPCFEGNKESGDVLIVAHGHILRAFAMRWTGRPLTETALILEAGGVGTLSYEHHNIEEPAIILGGGFVVEN

SEQ ID NO: 53 - DNA, 732 bp, Gibberella zeae
atgtcaaccccccgcgttttcctcattcgccatggcgagacggagtggtcgcttgatggtcgtcatactggtgtgactgacattcctcttactgccaatggcgagaaacgtgtgaaggcaacagggaaggctcttgtcggcccagaccgtctcattgctcccaagaagatcgctcatatctatgtctcaccccgaaagcgtgctcaacgcacatttgagctgctcaaccttgggctgagccgtccgctaccctggacaccacacggcgacacccctgatgggactggtctgcagtgtgaggccgaggttgaagtaacagactatatccgagaatgggactatggcgactacgaaggcattacatctccagagattcgcaagatcagagccgagcaggggatcaaggggtcctgggacatttggaaagatggttgtccaggtggagagagccctcatgatgtcagcaggagactcgatcaattgatagaagagattcgggataaatggcacaagccagtcatggaccaaggaagcgatcattgtggcgacgtcttacttgttgctcatggacacattcttcgtgcttttgccatgagatgggctggttacgctttgcgagaaggtccaacctttttactggaagcaggcggtgtcgggactctgaggatcgaagaaccagcgctcttgctgggcggcgcctttgttgttgaacttgacggccaagacaactag

SEQ ID NO: 54 - protein, 243 aa, Gibberella zeae
MSTPRVFLIRHGETEWSLDGRHTGVTDIPLTANGEKRVKATGKALVGPDRLIAPKKIAHIYVSPRKRAQRTFELLNLGLSRPLPWTPHGDTPDGTGLQCEAEVEVTDYIREWDYGDYEGITSPEIRKIRAEQGIKGSWDIWKDGCPGGESPHDVSRRLDQLIEEIRDKWHKPVMDQGSDHCGDVLLVAHGHILRAFAMRWAGYALREGPTFLLEAGGVGTLRIEEPALLLGGAFVVELDGQDN

SEQ ID NO: 55 - DNA, 582 bp, Rhizobium leguminosarum
atgagcagtgcgtttcccgagatctacttggtccgccacggtgaaaccgaatggagcctgtccgggcgccataccggacgcagtgatattcccttgacgtcgaatggcgaagaggccgcccgcaaaatcgccgaccggctggcgggccttagcttctccgccgtctggtcgagcccctccgagcgagcccgcaagacctgcgcgctcgccggattcggatcgggcgcggtgatcaaggacgatctcgccgaatgggactatggcgcttacgaaggcatcaccaccaaggcgatccttgccgaccgccccggctggcagctctttcgcgacggctgcccgaagggcgaattcgccgccgatgtcggcgatcgcgccgacgccgtgattggcggacttcgtcagacggccggcaccatcctgatcttttcgagttcgcatttcctgcgggtcctcgccgcccgctggcttggcctgccgccggaagacggcgcgcgtttcgtgctcgataccgcgagcatcagcgtgctcggctatgagcacgatctgaccgaaccggtcatccgccgctggaaccagagatag

SEQ ID NO: 56 - protein, 193 aa, Rhizobium leguminosarum
MSSAFPEIYLVRHGETEWSLSGRHTGRSDIPLTSNGEEAARKIADRLAGLSFSAVWSSPSERARKTCALAGFGSGAVIKDDLAEWDYGAYEGITTKAILADRPGWQLFRDGCPKGEFAADVGDRADAVIGGLRQTAGTILIFSSSHFLRVLAARWLGLPPEDGARFVLDTASISVLGYEHDLTEPVIRRWNQR

SEQ ID NO: 57 - DNA, 1665 bp, Ustilago maydis
atggcgatgcctcgcgtgttcctcgcgcgacacggcgagaccgaatggtccatctcgggccagcacactggtcgatccgatatcccactcaccccacacggtgaagaggtgatgcgacaacttgcgcccagcatcgtcggcgtcggcaacggcaagttgattgacccgactagactcaatcacatctttgtcagtccacgcaagcgatcgcagcgtacgcttgagatcatgctacagcacatccccgcctcgcagcgtttgggcatccctgacgtcgagatcgtccaggactgccgcgagtgggactacggtgcgtacgaagggctcaagacggacgagattcgagccaaacatcccggatgggatatctggaccgaaggaacacccgatcatcctgacagacccgacgagctgccaggtgagtcggcacagcacatgagcgatcgtgttgactcggtcatcgctaaaatccgcagtctgcaatctgggcatgtcgacaagcgcaatcagggtcacgatgtgggcagtaagacgtgcgatgttctacttgtatgccacggtcacttcaaccgggtgttcgttgctcgatggctcggtttaccgctgaccaacggacgtttgttcgaaatggatgcaggcggaatggtggtcctcggctatgcgcatcacagctttgctgaacctaccgtcgccggtatcttctcgtccaaaactggccccaagggtgacaaggaaggtgctcccggctcgtgtgggtcagcgaaacacgaagaatcgcagtacctcgagctggttcaacgagttatctcaacgggcgaatcaagaccggatcgaacgggcacaggtacgctcgcacttttcgctccgcaaccgagtttgcgattcgacttgacgaacggcaccttgcctctgctcaccaccaagcgagtgttcttccgcggagtgctggaggagttactctggttcgtgggtggtaaaaccgatgccaagcttctcaccgagaagagcgtgcacatttgggacggcaacggatctcgggccttcctcgactctcgtggactctcgcatcgtcgggaaggcgatctaggacccgtctatggtttccaatggcgacactttggtgctacgtacaaagactgtgataccgactacgccggtcagggtgtcgatcaactggcttccgtgatcgacaagattcgcaacaatcctaccgatcgtcgtatcttgctcagcgcatggaacccggcagatctcgaactgatggcgcttccgccatgccacatgtttgctcaattctttgtctcgaacctggacgcctattcgaagggtcaaggtaaaaagcacctctcgtgccagatgtaccagcgttcctgcgaccttgggttgggtgtacctttcaacattgctagctatgcgttgctaacctacatgatcgccaaagtgaccgattgcgaacccaaggaactcatactggcaatgggtgatgctcacgtttacaaggaccatgtgcaaccgctcaaggcgcagttggagagagacccgcttcccttccctaagctcaagatcgaaagggacgtcaaggatattgacgactttacgttcgatgatttcgtcgtgcaaggttacaaatgtcacggaaagatcgatatgaagatgagcgtttag

SEQ ID NO: 58 - protein, 554 aa, Ustilago maydis
MAMPRVFLARHGETEWSISGQHTGRSDIPLTPHGEEVMRQLAPSIVGVGNGKLIDPTRLNHIFVSPRKRSQRTLEIMLQHIPASQRLGIPDVEIVQDCREWDYGAYEGLKTDEIRAKHPGWDIWTEGTPDHPDRPDELPGESAQHMSDRVDSVIAKIRSLQSGHVDKRNQGHDVGSKTCDVLLVCHGHFNRVFVARWLGLPLTNGRLFEMDAGGMVVLGYAHHSFAEPTVAGIFSSKTGPKGDKEGAPGSCGSAKHEESQYLELVQRVISTGESRPDRTGTGTLALFAPQPSLRFDLTNGTLPLLTTKRVFFRGVLEELLWFVGGKTDAKLLTEKSVHIWDGNGSRAFLDSRGLSHRREGDLGPVYGFQWRHFGATYKDCDTDYAGQGVDQLASVIDKIRNNPTDRRILLSAWNPADLELMALPPCHMFAQFFVSNLDAYSKGQGKKHLSCQMYQRSCDLGLGVPFNIASYALLTYMIAKVTDCEPKELILAMGDAHVYKDHVQPLKAQLERDPLPFPKLKIERDVKDIDDFTFDDFVVQGYKCHGKIDMKMSV

SEQ ID NO: 59 - DNA, 1290 bp, Shigella flexneri
atggaaacgactcaaaccagcacgattgcgtcgaaagactctcgtagtgcctggcgcaagacagacaccatgtggatgctgggcctttacggcacggcaatcggcgcgggcgtgctgttcctgccaatcaacgccggtgttggcggtatgatcccgctgatcatcatggctatccttgcgttcccgatgacgttttttgctcaccgcggcctgactcgcttcgtactgtctggtaaaaacccgggcgaagacatcaccgaggttgtagaagaacactttggtattggcgcaggtaaactgattaccctgctctacttcttcgctatctacccgatcctgctggtttatagcgtggcaatcaccaataccgttgaaagcttcatgtctcaccagctgggtatgacgccaccgccgcgtgcgattctgtcgctgatcctgatcgtgggtatgatgaccatcgttcgctttggtgagcagatgatcgttaaagcgatgagtattctggtattcccgtttgttggcgtactgatgctgctggctctgtacctgatcccgcagtggaacggcgcagcactggaaacgctgtctctggacactgcatctgcaaccggaaacggtctgtggatgaccctgtggctggcaattccggtaatggtgttctcgttcaaccactctccgatcatctcttctttcgccgttgcgaatcgtgaagagtacggcgatatggcagaacagaaatgctccaagatcctggcattcgcacacatcatgatggtgctgaccgaaatgatcttcgtcttcatctgcgtactgagcctgactccggcagacctggctgcggctaaagagcacaacatctcgattctgtcttacctggctaaccactttaacgcaccggttatcgcgtggatggctccgattatcgcgattatcgctatcaccaaatccttcctcggccactacctgggcgcacgtgaaggcttcaacggtatggtgattaaatctctgcgtggtaaaggtaagtctatcgaaatcaacaagctgaaccgtatcactgcgctgttcatgctggtaacgacctggattgttgccaccctgaacccgagcatcctgggtatgattgaaaccctgggcggcccaatcatcgcgatgatcctgttcctgatgccgatgtacgcaattcagaaagtaccggcaatgcgtaagtacagcggtcacatcagcaacgtattcgttgtcgtgatgggtctgattgcaatctccgcaatcttctactctctgttcagctaa

SEQ ID NO: 60 - protein, 429 aa, Shigella flexneri
METTQTSTIASKDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFMSHQLGMTPPPRAILSLILIVGMMTIVRFGEQMIVKAMSILVFPFVGVLMLLALYLIPQWNGAALETLSLDTASATGNGLWMTLWLAIPVMVFSFNHSPIISSFAVANREEYGDMAEQKCSKILAFAHIMMVLTEMIFVFICVLSLTPADLAAAKEHNISILSYLANHFNAPVIAWMAPIIAIIAITKSFLGHYLGAREGFNGMVIKSLRGKGKSIEINKLNRITALFMLVTTWIVATLNPSILGMIETLGGPIIAMILFLMPMYAIQKVPAMRKYSGHISNVFVVVMGLIAISAIFYSLFS

SEQ ID NO: 61 - DNA, 1290 bp, Salmonella typhimurium
atggaaaccactcagaccagcactattgcttcgattgactctcgaagcgcatggcgcaaaacggataccatgtggatgctgggcctttacggcacggctatcggcgcgggcgttctgttcctgccgatcaacgcaggcgtcggcggcatgattccgctcatcatcatggcgattctcgctttcccgatgacctttttcgcacaccgtggtttaacccgcttcgtgctgtccggtaaaaatccgggtgaagacatcactgaagtcgttgaggaacacttcggtatcggcgcaggtaaactgattaccctgctttacttcttcgcgatttaccccatcctgctggtttacagcgtggcgattactaataccgtggaaagcttcctgacccaccagttagcgattaatccgccgccgcgggcgattctttccctgatcctgattgttggcatgatgaccatcgtgcgcttcggcgagcaaatgatcgttaaggcgatgagtatcctggtattcccgttcgtcgctgcgctgatgctgctggccttgtacctgatcccgcagtggaacggcgcggcgctggaaactctgtcctttgattccgccgcgtctaccggtaacggtctgtggatgacgctgtggctggcgattccggtgatggttttctctttcaaccactccccgatcatctcctccttcgcggttgcgaagcgtgaagagtacggtgaaggcgctgagaagaaatgttctaaaattctggccttcgcccacatcatgatggtgctgaccgtgatgttcttcgtcttcagctgcgtgctgagcctgacgccggcagatctggcagcggcaaaagagcagaacatctctattctgtcttacctggcgaaccattttaacgcgccgatcatcgcctggatggcgccgatcattgcgatgattgctatcaccaaatccttcctcggccactaccttggcgcgcgtgaaggctttaacgggatggtgattaaatcgctgcgtggtaaaggtaaatccatcgaaatcaacaaactgaataaaattaccgcgctgttcatgctggtcaccacctggattgtggcgacgctgaaccccagcatcctgggcatgattgaaaccctgggcggcccgatcatcgcgatgattctgttcctgatgccgatgtatgccatccagaaagtaccggcaatgcgtaagtacagcggtcatatcagcaacgtattcgttgtgattatgggcctgattgccatctccgctattttctactcactgttcagctaa

SEQ ID NO: 62 - protein, 429 aa, Salmonella typhimurium
METTQTSTIASIDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFLTHQLAINPPPRAILSLILIVGMMTIVRFGEQMIVKAMSILVFPFVAALMLLALYLIPQWNGAALETLSFDSAASTGNGLWMTLWLAIPVMVFSFNHSPIISSFAVAKREEYGEGAEKKCSKILAFAHIMMVLTVMFFVFSCVLSLTPADLAAAKEQNISILSYLANHFNAPIIAWMAPIIAMIAITKSFLGHYLGAREGFNGMVIKSLRGKGKSIEINKLNKITALFMLVTTWIVATLNPSILGMIETLGGPIIAMILFLMPMYAIQKVPAMRKYSGHISNVFVVIMGLIAISAIFYSLFS

SEQ ID NO: 63 - DNA, 1290 bp, Salmonella enterica
atgaaaaccactcagaccagcacgattgcttcgattgactctcgaagcgcatggcgcaaaacggataccatgtggatgctgggcctttacggcacggcaatcggcgcgggcgttctgttcctgccgatcaacgcaggcgtcggcggcatgattccgctcatcatcatggcgattctcgctttcccgatgacctttttcgcacaccgtggtttaacccgcttcgtgctgtccggtaaaaacccgggtgaagacatcactgaagtcgttgaggaacacttcggtatcggcgcaggtaaactgattaccctgctctacttcttcgcgatttaccccatcctgctggtttacagcgtggcgattactaataccgtggaaagcttcctgacccaccagttagcgattaatccgccgccgcgggcgattctttccctgatcctgattgttggcatgatgaccatcgtgcgcttcggcgagcaaatgatcgttaaggcgatgagtatcctggtattcccgttcgtcgctgcgctgatgctgctggccttgtacctgatcccgcagtggaacggcgcggcgctggaaactctgtcctttgattccgccgcgtctaccggtaacggtctgtggatgacgctgtggctggcgattccggtgatggttttctctttcaaccactccccgatcatctcctccttcgcggttgcgaagcgtgaagagtacggtgaaggcgctgagaagaaatgttctaaaattctggccttcgcccacatcatgatggtgctgaccgtgatgttcttcgtcttcagctgcgtgctgagcctgacgccggcagatctggcagcggcaaaagagcagaacatctctattctgtcttacctggcgaaccactttaacgcgccgatcatcgcctggatggcgccgatcattgcgatgattgctatcaccaaatccttcctcggccactacctgggcgcgcgtgaaggctttaacgggatggtgattaaatcgctgcgtggtaaaggtaaatccatcgaaatcaacaaactgaataaaattaccgcgctgttcatgctggtcaccacctggattgtggcgacgctgaaccccagcatcctgggcatgattgaaaccctgggcggcccgatcatcgcgatgattctgttcctgatgccgatgtatgccatccagaaagtaccggcaatgcgtaagtacagcggtcatatcagcaacgtattcgttgtgattatgggcctgattgccatctccgctattttctactcactgttcagctaa

SEQ ID NO: 64 - protein, 429 aa, Salmonella enterica
MKTTQTSTIASIDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFLTHQLAINPPPRAILSLILIVGMMTIVRFGEQMIVKAMSILVFPFVAALMLLALYLIPQWNGAALETLSFDSAASTGNGLWMTLWLAIPVMVFSFNHSPIISSFAVAKREEYGEGAEKKCSKILAFAHIMMVLTVMFFVFSCVLSLTPADLAAAKEQNISILSYLANHFNAPIIAWMAPIIAMIAITKSFLGHYLGAREGFNGMVIKSLRGKGKSIEINKLNKITALFMLVTTWIVATLNPSILGMIETLGGPIIAMILFLMPMYAIQKVPAMRKYSGHISNVFVVIMGLIAISAIFYSLFS

SEQ ID NO: 65 - DNA, 1302 bp, Yersinia pseudotuberculosis
atggacactacgcaaacaagcactcttgcctcggcaagaaaaatctcatcaagtacatggcgcaaaagtgacaccatgtggatgttgggtctgtacggtacagcgattggcgcaggtgtactctttttgcccatcaacgccggtattggtggcctgctaccactgattgttatggctatcattgctttcccaatgacctactacgctcaccgtggcctgtgccgattcgtgttatccggtaaaaatccaggtgaagatattactgaggtggttgaagaacatttcggtgtaggcgcaggtaaactgattaccctgctttacttcttcgctatctaccccattctcttggtgtacagcgtagcaattaccaataccgtggatagtttcattacacatcagatgcacctgccatcaccaccacgcgccattttgtccctgattctgattgtgggcctgatggctatcgtgcgctttggtgaacagaccatcgtcaaagccatgagcatcttggtattcccgtttgttgccgtattgatgatgttggcgatatacctgatcccaaactggtcaggtgcaatattcgaaaacgtatccatggacggtaacggcacaggtagcggcctgtggatgacaatgtggctaatcattcccgtcatggtgttctctttcaaccactcaccgatcatctcagcctttgctgtagcaaagcgcgaagagtacggggtggatgcagagaaaaaatgttcacgcattttgagctttgcccacatcatgatggttatcaccgtcatgttcttcgtcttcagttgcgtactgagcctgtctcctgcggatctgatggatgcaaaaaaccaaaacatctccattttgtcttatctggcaaaccattttaatacgccagtaattgcctatatggcaccggttattgcctttattgccatcactaaatcattcctgggccactatctgggcgcccgtgaaggttttaacggtatggtcattaagtctctgcgtagtagaggcaaaaccattgagcagaacaaactgaaccgcattactgcgttgttcatgttagtcaccacctgggctgtcgcgaccttaaacccaagtatcttggggatgatcgaaacattgggcggccctatcatcgcgatgctcctgttcctgatgccgatgtatgccattcataaagtcccggccatgcgtaaatacagcggccagatcagtaacgtattcgtggtactcatgggcttgatcgccatctctgggatcttattcaacttaagcaacttgtttaattag

SEQ ID NO: 66 - protein, 433 aa, Yersinia pseudotuberculosis
MDTTQTSTLASARKISSSTWRKSDTMWMLGLYGTAIGAGVLFLPINAGIGGLLPLIVMAIIAFPMTYYAHRGLCRFVLSGKNPGEDITEVVEEHFGVGAGKLITLLYFFAIYPILLVYSVAITNTVDSFITHQMHLPSPPRAILSLILIVGLMAIVRFGEQTIVKAMSILVFPFVAVLMMLAIYLIPNWSGAIFENVSMDGNGTGSGLWMTMWLIIPVMVFSFNHSPIISAFAVAKREEYGVDAEKKCSRILSFAHIMMVITVMFFVFSCVLSLSPADLMDAKNQNISILSYLANHFNTPVIAYMAPVIAFIAITKSFLGHYLGAREGFNGMVIKSLRSRGKTIEQNKLNRITALFMLVTTWAVATLNPSILGMIETLGGPIIAMLLFLMPMYAIHKVPAMRKYSGQISNVFVVLMGLIAISGILFNLSNLFN

SEQ ID NO: 67 - DNA, 1302 bp, Yersinia pestis
atggacactacgcaaacaagcactcttgcctcggcaagaaaaatctcatcaagtacatggcgcaaaagtgacaccatgtggatgttgggtctgtacggtacagcgattggcgcaggtgtactctttttgcccatcaacgccggtattggtggcctgctaccactgattgttatggctatcattgctttcccaatgacctactacgctcaccgtggcctgtgccgattcgtgttatccggtaaaaatccaggtgaagatattactgaggtggttgaagaacatttcggtgtaggcgcaggtaaactgattaccctgctttacttcttcgctatctaccccattctcttggtgtacagcgtagcaattaccaataccgtggatagtttcattacacatcagatacacctgccatcaccaccacgcgccattttgtccctgattctgattgtgggcctgatggctatcgtgcgctttggtgaacagaccatcgtcaaagccatgagcatcttggtattcccgtttgttgccgtattgatgatgttggcgatatacctgatcccaaactggtcaggtgcaatattcgaaaacgtatccatggacggtaacggcacaggtagcggcctgtggatgacaatgtggctaatcattcccgtcatggtgttctctttcaaccactcaccgatcatctcagcctttgctgtagcaaagcgcgaagagtacggggtggatgcagagaaaaaatgttcacgcattttgagctttgcccacatcatgatggttatcaccgtcatgttcttcgtcttcagttgcgtactgagcctgtctcctgcggatctgatggatgcaaaaaaccaaaacatctccattttgtcttatctggcaaaccattttaatacgccagtaattgcctatatggcaccggttattgcctttattgccatcactaaatcattcctgggccactatctgggcgcccgtgaaggttttaacggtatggtcattaagtctctgcgtagtagaggcaaaaccattgagcagaacaaactgaaccgcattactgcgttgttcatgttagtcaccacctgggctgtcgcgaccttaaacccaagtatcttggggatgatcgaaacattgggcggccctatcatcgcgatgctcctgttcctgatgccgatgtatgccattcataaagtcccggccatgcgtaaatacagcggccagatcagtaacgtattcgtggtactcatgggcttgatcgccatctctgggatcttattcaacttaagcaacttgtttaattag

SEQ ID NO: 68 - protein, 433 aa, Yersinia pestis
MDTTQTSTLASARKISSSTWRKSDTMWMLGLYGTAIGAGVLFLPINAGIGGLLPLIVMAIIAFPMTYYAHRGLCRFVLSGKNPGEDITEVVEEHFGVGAGKLITLLYFFAIYPILLVYSVAITNTVDSFITHQIHLPSPPRAILSLILIVGLMAIVRFGEQTIVKAMSILVFPFVAVLMMLAIYLIPNWSGAIFENVSMDGNGTGSGLWMTMWLIIPVMVFSFNHSPIISAFAVAKREEYGVDAEKKCSRILSFAHIMMVITVMFFVFSCVLSLSPADLMDAKNQNISILSYLANHFNTPVIAYMAPVIAFIAITKSFLGHYLGAREGFNGMVIKSLRSRGKTIEQNKLNRITALFMLVTTWAVATLNPSILGMIETLGGPIIAMLLFLMPMYAIHKVPAMRKYSGQISNVFVVLMGLIAISGILFNLSNLFN

SEQ ID NO: 69 - DNA, 1323 bp, Yersinia pestis
gtgttcacaatggagattttcatggacactacgcaaacaagcactcttgcctcggcaagaaaaatctcatcaagtacatggcgcaaaagtgacaccatgtggatgttgggtctgtacggtacagcgattggcgcaggtgtactctttttgcccatcaacgccggtattggtggcctgctaccactgattgttatggctatcattgctttcccaatgacctactacgctcaccgtggcctgtgccgattcgtgttatccggtaaaaatccaggtgaagatattactgaggtggttgaagaacatttcggtgtaggcgcaggtaaactgattaccctgctttacttcttcgctatctaccccattctcttggtgtacagcgtagcaattaccaataccgtggatagtttcattacacatcagatacacctgccatcaccaccacgcgccattttgtccctgattctgattgtgggcctgatggctatcgtgcgctttggtgaacagaccatcgtcaaagccatgagcatcttggtattcccgtttgttgccgtattgatgatgttggcgatatacctgatcccaaactggtcaggtgcaatattcgaaaacgtatccatggacggtaacggcacaggtagcggcctgtggatgacaatgtggctaatcattcccgtcatggtgttctctttcaaccactcaccgatcatctcagcctttgctgtagcaaagcgcgaagagtacggggtggatgcagagaaaaaatgttcacgcattttgagctttgcccacatcatgatggttatcaccgtcatgttcttcgtcttcagttgcgtactgagcctgtctcctgcggatctgatggatgcaaaaaaccaaaacatctccattttgtcttatctggcaaaccattttaatacgccagtaattgcctatatggcaccggttattgcctttattgccatcactaaatcattcctgggccactatctgggcgcccgtgaaggttttaacggtatggtcattaagtctctgcgtagtagaggcaaaaccattgagcagaacaaactgaaccgcattactgcgttgttcatgttagtcaccacctgggctgtcgcgaccttaaacccaagtatcttggggatgatcgaaacattgggcggccctatcatcgcgatgctcctgttcctgatgccgatgtatgccattcataaagtcccggccatgcgtaaatacagcggccagatcagtaacgtattcgtggtactcatgggcttgatcgccatctctgggatcttattcaacttaagcaacttgtttaattag

SEQ ID NO: 70 - protein, 440 aa, Yersinia pestis
MFTMEIFMDTTQTSTLASARKISSSTWRKSDTMWMLGLYGTAIGAGVLFLPINAGIGGLLPLIVMAIIAFPMTYYAHRGLCRFVLSGKNPGEDITEVVEEHFGVGAGKLITLLYFFAIYPILLVYSVAITNTVDSFITHQIHLPSPPRAILSLILIVGLMAIVRFGEQTIVKAMSILVFPFVAVLMMLAIYLIPNWSGAIFENVSMDGNGTGSGLWMTMWLIIPVMVFSFNHSPIISAFAVAKREEYGVDAEKKCSRILSFAHIMMVITVMFFVFSCVLSLSPADLMDAKNQNISILSYLANHFNTPVIAYMAPVIAFIAITKSFLGHYLGAREGFNGMVIKSLRSRGKTIEQNKLNRITALFMLVTTWAVATLNPSILGMIETLGGPIIAMLLFLMPMYAIHKVPAMRKYSGQISNVFVVLMGLIAISGILFNLSNLFN

SEQ ID NO: 71 - DNA, 1290 bp, Erwinia carotovora
atgagcacaattcaagatagcagccacgtgttagaacaggcttcaggatggcgtaaaagcgacaccgtctggatgctcggcctctatggcacggccatcggtgcgggcgtgttattcttacccatcaatgctggcattggtggtttaattccgttaattatcatggcactgattgcctttccgatgacgtattattctcaccgtgcattatgccgctttgtattatccggcaagaaaggtggcgaagatattaccgaagtcgttgaagaacatttcggcgttggtgcgggtaaattaatcactttgctttattttttcgctatttatccgatcctattagtttatagcgtcgctatcaccaatacggtagatagctttattactcaccaattgcatttaccttctccgccgcgtgctgcgctgtcattaatattgatcctcgggttgatgtttattgtccgcttcggtgaagcgatgatcgttaaagcaatgagtgttctggtttacccttttgttgccgtactgatgttgctggcgctttatttaattccgaactggaatacctctgtttttgaaaacatttctctgagttccagcgtaacgggtaacggtctgttggcgacattatggctggctattccggtgatggttttctccttcaaccactcgccaatcatctcctctttcgccgttgccaaacgcaaagaatatggtgatgatgcagagaagaaatgctcacgcattctgtcttacagccacattatgatggtgttgacggtgatgttcttcgttttcagctgcgtactgagcctttctccaaccgagctgatggaagcaaaaacacaaaacatttctattctgtcctatctggcgaaccattttaatagcccggtaatcggctatctggccccagtaattgcaaccatcgcgatctctaaatccttcctgggccactatctgggcgcgggtgaaggctttaacggcatgatcgtgaaatccctgcgcagcagaggaaaaaccatctccagcaccaaactgaaccgtatcacttcgctgttcatgttgatcaccacctggattgtcgcgacgctgaacccaagcattctgggtatgatcgaaaccttgggcggcccgattatcgcgtgcctgctgttcctgatgccgatgtacgccatccagaaagtcccagcgatgaaaaaatacagcggccacattagcaacgtctttgtcaccctcatgggactgatcgctatttccgccattgtttacagcctgcgcgatctgctgtaa

SEQ ID NO: 72 - protein, 429 aa, Erwinia carotovora
MSTIQDSSHVLEQASGWRKSDTVWMLGLYGTAIGAGVLFLPINAGIGGLIPLIIMALIAFPMTYYSHRALCRFVLSGKKGGEDITEVVEEHFGVGAGKLITLLYFFAIYPILLVYSVAITNTVDSFITHQLHLPSPPRAALSLILILGLMFIVRFGEAMIVKAMSVLVYPFVAVLMLLALYLIPNWNTSVFENISLSSSVTGNGLLATLWLAIPVMVFSFNHSPIISSFAVAKRKEYGDDAEKKCSRILSYSHIMMVLTVMFFVFSCVLSLSPTELMEAKTQNISILSYLANHFNSPVIGYLAPVIATIAISKSFLGHYLGAGEGFNGMIVKSLRSRGKTISSTKLNRITSLFMLITTWIVATLNPSILGMIETLGGPIIACLLFLMPMYAIQKVPAMKKYSGHISNVFVTLMGLIAISAIVYSLRDLL

SEQ ID NO: 73 - DNA, 1299 bp, Photorhabdus luminescens
atggatatgactcaaacaggtacgatcgcatcgcaggcgaccagcccaagcgattataaaacctggcgtaaatcagatacagtatggatgcttggcttatacggtactgcgattggtgccggtgtattattcttacccatcaatgctggtattggtggcctactacctcttttggtaatggcgctactggctttcccaatgactttcttcgcccaccgcgggatgtgtcgtttcgttctatcaggtaaaaaccctggcgaagatattaccgaagtcgtagaagaacactttggcaacctggcaggtaaacttattactctgctctacttcttcgctatctacccgattctcttagtttacagcgttgctattaccaacaccgtagacagctttattgttcaccaattacacctgccatcacctcctcgtgctctgctggctctgatcctaattactggtgtaatgaccatcatccgttttggtgaacaggctattgttaaggcaatgagcgtgctggtattcccatttgttgccgttttgatgctactggcgttttatctgatccctaactggaataccgcaatcttcgaaaacatcagcgtttctgcatcgagtgcaagtcatgggctgctcattactctatggctagcgatcccagtaatggtgttctccttcaaccactcgccaattatctcggcatttgcggtggcaaaacgtggagaatatggtgagcatgctgagaaaaaatgctcccgcattctagcttacgcccatatcatgatggttgtaaccgtgatgttcttcgtattcagctgcgtattaagcctgtctccagaaaatctggcagaagccaaagcacaaaatatttctattctgtcttacctggctaaccactttaatacaccagtggttgcctacatcgctccgtttattgcattcatagcaattaccaaatctttccttggccattatctgggtgcacgtgaaggttttaatggtattgtggttaagacattgcgtgaccaaggtaaaactattgagcctaaaaagctgaatcgtatcactaccattttcatgttagtaacgacttggattgtcgctactctgaacccaagtattttggaaatgatcgaaagattaggtggtcctattatcgcaatgttgttgttcattatgccaatgtatgcaatcagcaaagttcctgcaatgcgcaaatatagcggcaagctgagtaatctgtttgtcgtctttatgggattagttgctatctctgctgtcctctactcactgtttgcataa

SEQ ID NO: 74 - protein, 432 aa, Photorhabdus luminescens
MDMTQTGTIASQATSPSDYKTWRKSDTVWMLGLYGTAIGAGVLFLPINAGIGGLLPLLVMALLAFPMTFFAHRGMCRFVLSGKNPGEDITEVVEEHFGNLAGKLITLLYFFAIYPILLVYSVAITNTVDSFIVHQLHLPSPPRALLALILITGVMTIIRFGEQAIVKAMSVLVFPFVAVLMLLAFYLIPNWNTAIFENISVSASSASHGLLITLWLAIPVMVFSFNHSPIISAFAVAKRGEYGEHAEKKCSRILAYAHIMMVVTVMFFVFSCVLSLSPENLAEAKAQNISILSYLANHFNTPVVAYIAPFIAFIAITKSFLGHYLGAREGFNGIVVKTLRDQGKTIEPKKLNRITTIFMLVTTWIVATLNPSILEMIERLGGPIIAMLLFIMPMYAISKVPAMRKYSGKLSNLFVVFMGLVAISAVLYSLFA

SEQ ID NO: 75 - DNA, 1299 bp, Chromobacterium violaceum
atgtctcaagcaatcgcctccaccgcttcccgctccggttcatccggctggtccaagcacgacaccacctggatgctcggcctgtacggcaccgccatcggcgccggcgtcctgttcctcccgatcaacgccggcatcggcggcctgtggccgctgatgctgatggccatcctggccctgccgctgaccttcttcgcccaccgcggcctgacccgcttcgtgctgtccggcagcaaggaaggcgcggacatcaccgaggtggtggaagagcacttcggtctcggcgccggcaagatcatcaccctgctgtacttcttcgccatctacccgatcctgctgatgtacagcgtggccatcaccaacacggtgctgtccttcctgaacaaccagctgcacatcgaagtgggcaccggcatcgccacccgcgccgtgttctcgctggcgctgatcctgggcctgatgtccatcgtgcgcctgggcggccagatgatcgtcaaggcgatgagcatcctggtgtacccgttcgtggtggtgctgatgctgctggcgctgtacctgatcccgcaatggagcgataccgccatccgccacgccggcagcctgggcgacgcgctgtccagcggcgccttctacaagacgctgtggctggccatcccggtgatggtgttctcgttcaaccactcgccgatcatctcgtccttctccgtcgaccagcgcaagcttcacggcgatgacgccgagccggcctccagccgcgtgctgattcgcgcccacaccatgatggtgctgaccgtgatgttcttcgtgttcagctgcgtgttcagcctgagcccggccgacctggccgccgccaaggcccagaacatctccatcctgtcctacctggccaaccacttccaaaacccggtgatggaatgggttgccccgatcatcgccatggtcgccatcagcaagtccttcctgggccactacctgggcgccaaggaaggcttcaacggcctggtgatcaagcagctgcgccagaacggcaagagcatcgaatcgtccaagctggaccgctacaccgccatcttcatgatcgtcacctgctggatcatcgccaccatcaacccgtccatcctgggcatgatcgaaaccctgggcggcccggtgatcgcgatgctgctgttcctgatgccgatgtacgcgatccagaaagtgccggcgatgaagaagtactccggcgccgccagcaacatcttcgtgacgctgatcggcctgatcgccatctccgccatcttctacgatctgatcgcctga

SEQ ID NO: 76 - protein, 432 aa, Chromobacterium violaceum
MSQAIASTASRSGSSGWSKHDTTWMLGLYGTAIGAGVLFLPINAGIGGLWPLMLMAILALPLTFFAHRGLTRFVLSGSKEGADITEVVEEHFGLGAGKIITLLYFFAIYPILLMYSVAITNTVLSFLNNQLHIEVGTGIATRAVFSLALILGLMSIVRLGGQMIVKAMSILVYPFVVVLMLLALYLIPQWSDTAIRHAGSLGDALSSGAFYKTLWLAIPVMVFSFNHSPIISSFSVDQRKLHGDDAEPASSRVLIRAHTMMVLTVMFFVFSCVFSLSPADLAAAKAQNISILSYLANHFQNPVMEWVAPIIAMVAISKSFLGHYLGAKEGFNGLVIKQLRQNGKSIESSKLDRYTAIFMIVTCWIIATINPSILGMIETLGGPVIAMLLFLMPMYAIQKVPAMKKYSGAASNIFVTLIGLIAISAIFYDLIA

SEQ ID NO: 77 - DNA, 1290 bp, Photobacterium sp.
atgggcaccgaacaaaatttaacggcaacggcttctgcacaaagcaaaaaattagggtggagcaaaagtgatacattatggatgcttggtctatacggtacagccgtaggcgcaggtactctatttttaccaattaatgcgggggttggtggtttaattccattacttgtaatggccgttctcgcttttccaatgacattctttgcccaccgtgcaatgacacgttttgtattatcaagttcgaatcctggcgctgatattacagaagttgttgaagagcactttggtaagggcatgggtaaagtgatcacattactttatttctttgctatctaccctatcctattagtttacagtgttgcactaacaaatactgttgaaagctttatgcttaaccagttaggtatcgaaccaccagcacgtgcaattttagcgttagtattgatccttggtttaatggcaattgttcgtttaggtgagcaattaattgttaaagcgatgagtatcttggtattcccattcgttgctgtattattaattcttgcactatttcttgtaccgtactggaatgactctatcttccacaacgttatgccagcagacggtggcttaaactcagtgatgcttgcagtatggctaattttaccggtaatggttttctcgtttaaccactcacctgttatctcatcatttgctgttgctaagcaaaaagaatacggcgtaaacgcagagcgtcaaagttcacgtattctagcgcgtgctcacattatgatggtattaaccgtaatgttcttcgttttcagctgtgtattaagcttatcacctgaaaacttagcagaagctaaagcgaataacgtatctattctgacttacttagcgaaccacttcgatacaccgatcattgcttatgttgcaccgattgtagcgattattgcaattactaagtctttccttggccactacttaggtgcaagtgaaggtctaaatggtttagttatcaaagttactcgtgataagaataaagagatctcaagtaaggcactaaaccgttttaccgcaatctttatgctagtaacaacatgggcagtagcaacactaaacccaagcattctaggtatgattgagagcttaggtggtcctatcattgcaatgttgctatttattatgccaatgtatgcaatcaaaaaagtaccagcgatgaagaaatactcaggcgcaatcagtaatgtatttgtgacggttatcggacttgtttctatctctgcaatcttctactcattatttatgtaa

SEQ ID NO: 78 - protein, 429 aa, Photobacterium sp.
MGTEQNLTATASAQSKKLGWSKSDTLWMLGLYGTAVGAGTLFLPINAGVGGLIPLLVMAVLAFPMTFFAHRAMTRFVLSSSNPGADITEVVEEHFGKGMGKVITLLYFFAIYPILLVYSVALTNTVESFMLNQLGIEPPARAILALVLILGLMAIVRLGEQLIVKAMSILVFPFVAVLLILALFLVPYWNDSIFHNVMPADGGLNSVMLAVWLILPVMVFSFNHSPVISSFAVAKQKEYGVNAERQSSRILARAHIMMVLTVMFFVFSCVLSLSPENLAEAKANNVSILTYLANHFDTPIIAYVAPIVAIIAITKSFLGHYLGASEGLNGLVIKVTRDKNKEISSKALNRFTAIFMLVTTWAVATLNPSILGMIESLGGPIIAMLLFIMPMYAIKKVPAMKKYSGAISNVFVTVIGLVSISAIFYSLFM

SEQ ID NO: 79 - DNA, 1302 bp, Pseudomonas fluorescens
atgaccgatgtacgcacacctgctgccgaaaatcccgctgtagaccgcacacgcaataacgaaaccgcccacaagggctggagcaagttcgacaccacctggatgctcggcctgtatggcaccgccattggcgccggcaccctgttcctgccgatcaacgccggtgtcggcggcttctggccgttgctgatcctggcgttgctggctttcccgatgaccttcttcgcgcaccgtggcctcacccgcttcgtgctctcgggccgctccggtgacatcaccgaagtggtggaggaacacttcggcatcggcgccggcaaactgattacgctgctgtatttcttcgcaatcttcccgatcctgctggtgtacagcgtggcgctgaccaacactctcggcagcttcctcgaacaccagttgcacatcgccccgccaccgcgcgcggtgctgtcgctggcgctgatcctcggtctgatggccatcgtccgttgcggtcagaacgtgatcgtcaaagccatgagtgtgctggtgtatccgttcgtcgccgcgttgctgttgctcggcatcagcctgattccgaactggaacggcgcattcttcgccagcgctcaagaaccgatggaaatgtcggtgttcctcaagaccctgtggctggcgatcccggtgatggtgttctcgttcaaccattcacccatcatctctgcgttcgccgtcgagcagaaacagcgctacggcgagcaggccgaacgcaagagcagcggcatcctcgccatggcccacggcatgatggtcgtcacggtgatgttcttctgcttcagctgcgtgctggcgctgtcgccgacggatctggcagcggccaaggcgcagaacatctcgatcctgtcgtatctggccaaccacttccagaccccggtcatcgcttacgccgcgccattgattgcgctggtggcgatcaccaaatccttcctcggccactacatcggcgccagcgaaggcttccagggcatgatcgtcaaaagcctgcgcagccgtggccgggtgatgtcggcgagctggctgaaccgtgcgaccgcgctgttcatgatcctcagctgctgggccgtggcgaccttcaacccgagcatcctcggcatgatcgaaaccctcggcgggccggtgattgcctgcctgctgttcctgatgccgatgtacgcgatccgccgcgtgccagccttgcgccagtattcgggccaggtgtcgaacgtgttcgtggtgctgatcggcctgattgcactgtcagcgatcatctactcggttctgccctga

SEQ ID NO: 80 - protein, 433 aa, Pseudomonas fluorescens
MTDVRTPAAENPAVDRTRNNETAHKGWSKFDTTWMLGLYGTAIGAGTLFLPINAGVGGFWPLLILALLAFPMTFFAHRGLTRFVLSGRSGDITEVVEEHFGIGAGKLITLLYFFAIFPILLVYSVALTNTLGSFLEHQLHIAPPPRAVLSLALILGLMAIVRCGQNVIVKAMSVLVYPFVAALLLLGISLIPNWNGAFFASAQEPMEMSVFLKTLWLAIPVMVFSFNHSPIISAFAVEQKQRYGEQAERKSSGILAMAHGMMVVTVMFFCFSCVLALSPTDLAAAKAQNISILSYLANHFQTPVIAYAAPLIALVAITKSFLGHYIGASEGFQGMIVKSLRSRGRVMSASWLNRATALFMILSCWAVATFNPSILGMIETLGGPVIACLLFLMPMYAIRRVPALRQYSGQVSNVFVVLIGLIALSAIIYSVLP

SEQ ID NO: 81 - DNA, 1257 bp, Vibrio parahaemolyticus
gtgaaagaatctcgaaatacgttaaattctagtgaattaaacacaacaacctcttggtcaaaacatgacacacattgggtacttagcctattcgggaccgccgttggtgccggtattttgtttctgccaatcaacttgggcattggtggattttggccactagtcgcaatggcgtttttggcatttccaatgacttacctcgcccatcgcggtttagcgcgctttgtattgtcttctaagatcaaaaatgccgactttaccgatgtggttgaagaacactttggcgcaaaagcgggacgttcaatctctctgctttatttcctatcgatttttcctatcctgttaatttacggcgtgggcatcaccaatactgtagacagctttatggtgaaccaagcgggaatggaagccttaccacgcgagctactttcgggcgtattagtgttcgctttgattgccattatgatggcgggcgaaaaagtgatgctgcgcgcgtttgccgttatggtgtacccacttgttgcgattctagctttcctctcattctacttaatgccgaactggacaatgcccgtgcttgatacaccggacatgggtgcgtttgcgagcacaatgtggttagccgttcctgttgtgattttctcgtttagccatgcagcagcgatctctagctttgccaatgttcagcgccgacactacggcgatgatgccgatgcaaaagccgaactgattttacgttgcacgagcatcatgcttatcgcattcgtactattgtttgttttctcgtgcgtattagcactttcgccagagcaacttgcacaggcaaaagcgcaaaacgtatccgtactctcttaccttgcaaacgcaacggataacccattcatcgcaacacttggtcctttggtggcgtttgtcgcgatcacttcttctttccttggtcatttcttaggcgcacgtgaaagcttgaacggcttaatcacaaagcactcaaatttgtctgaaacacgtgttgatcgtattagcgtagtggttcttttcttgtcaatttgggctgcagcaatcatgaacccaagtattttaggcatgatggaagcgttatctgggccagttatcgcaatgatcttgttcattatgccgatgttggcggtacataagattgagtcgatgaagcagtatcgtggaaagctctctacctattttgttctgatcacaggtattgttgcggtaagtgctttggtttttagcttactcagctaa

SEQ ID NO: 82 - protein, 418 aa, Vibrio parahaemolyticus
MKESRNTLNSSELNTTTSWSKHDTHWVLSLFGTAVGAGILFLPINLGIGGFWPLVAMAFLAFPMTYLAHRGLARFVLSSKIKNADFTDVVEEHFGAKAGRSISLLYFLSIFPILLIYGVGITNTVDSFMVNQAGMEALPRELLSGVLVFALIAIMMAGEKVMLRAFAVMVYPLVAILAFLSFYLMPNWTMPVLDTPDMGAFASTMWLAVPVVIFSFSHAAAISSFANVQRRHYGDDADAKAELILRCTSIMLIAFVLLFVFSCVLALSPEQLAQAKAQNVSVLSYLANATDNPFIATLGPLVAFVAITSSFLGHFLGARESLNGLITKHSNLSETRVDRISVVVLFLSIWAAAIMNPSILGMMEALSGPVIAMILFIMPMLAVHKIESMKQYRGKLSTYFVLITGIVAVSALVFSLLS

SEQ ID NO: 83 - DNA, 1425 bp, Shigella flexneri
atgaaaaaaaacacattagcactgagtgcactggctctgagtttaggtttggcgttatctccgctctctgcaacggcggctgagacttcttcagcaacgacagcccagcagatgccaagccttgcaccgatgctcgaaaaggtgatgccttcagtggtcagcattaacgtagaaggtagcacaaccgttaatacgccgcgtatgccgcgtaatttccagcagttcttcggtgatgattctccgttctgccaggaaggttctccgttccagagctctccgttctgccaggggggccagggcggtaatggcggcggccagcaacagaaattcatggcgctgggttccggcgtcatcattgatgccgataaaggctatgtcgtcaccaacaaccacgttgttgataacgcgacggtgattaaagtccagctgagcgatggtcgtaagttcgacgcgaagatggttggcaaagatccgcgctctgatatcgcgctgatccaaatccagaacccgaaaaaactgaccgcaattaagatggcggattctgatgcactgcgcgtgggtgattacaccgtagcgattggtaacccgtttggtctgggcgagacggtaacttccgggattgtctctgcgctggggcgtagcggcctgaatgctgaaaactacgaaaacttcatccagaccgatgcagcgatcaaccgtggtaactccggtggtgcgctggttaacctgaacggcgaactgatcggtatcaacaccgcgatcctcgcaccggacggcggcaacatcggtatcggttttgctatcccgagtaacatggtgaaaaacctgacctcgcagatggtggaatacggccaggtgaaacgcggtgagctgggtattatggggactgagctgaattccgaactggcgaaagcgatgaaagttgacgcccagcgcggtgctttcgtaagccaggttctgcctaattcctccgctgcaaaagcgggcattaaagcgggtgatgtgatcacctcactgaacggtaagccgatcagcagctttgccgcactgcgtgctcaggtgggtactatgccggtgggcagcaaactgaccctgggcttactgcgcgacggtaagcaggttaacgtgaacctggaactgcagcagagcagccagaatcaggttgattccagctccatcttcaacggcattgaaggcgctgagatgagcaacaaaggcaaagatcagggcgtggtagtgaacaacgtgaagacgggcactccggctgcgcagatcggcctgaagaaaggtgatgtgattattggtgcgaaccagcaggcagtgaaaaacatcgctgaactgcgtaaagttctcgacagcaaaccgtctgtgctggcactgaacattcagcgcggcgacagcaccatctacctgttaatgcagtaa

SEQ ID NO: 84 - protein, 474 aa, Shigella flexneri
MKKNTLALSALALSLGLALSPLSATAAETSSATTAQQMPSLAPMLEKVMPSVVSINVEGSTTVNTPRMPRNFQQFFGDDSPFCQEGSPFQSSPFCQGGQGGNGGGQQQKFMALGSGVIIDADKGYVVTNNHVVDNATVIKVQLSDGRKFDAKMVGKDPRSDIALIQIQNPKKLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVKNLTSQMVEYGQVKRGELGIMGTELNSELAKAMKVDAQRGAFVSQVLPNSSAAKAGIKAGDVITSLNGKPISSFAALRAQVGTMPVGSKLTLGLLRDGKQVNVNLELQQSSQNQVDSSSIFNGIEGAEMSNKGKDQGVVVNNVKTGTPAAQIGLKKGDVIIGANQQAVKNIAELRKVLDSKPSVLALNIQRGDSTIYLLMQ

SEQ ID NO: 85 - DNA, 1425 bp, Escherichia coli
atgaaaaaaaccacattagcactgagtgcactggctctgagtttaggtttggcgttatctccgctctctgcaacggcggctgagacttcttcagcaacgacagcccagcagatgccaagccttgcaccgatgctcgaaaaggtgatgccttcagtggtcagcattaacgtagaaggtagcacaaccgttaatacgccgcgtatgccgcgtaatttccagcagttcttcggtgatgattctccgttctgccaggagggttctccgttccagagctctccgttctgccaggggggcctgggcggtaatggcggcggccagcaacagaaattcatggcgctgggttccggcgttatcattgatgccgataaaggctatgtcgtcaccaacaaccacgttgttgataacgcgacggtgattaaagtccaactgagcgatggtcgtaagttcgacgcgaagatggttggcaaagatccgcgctctgatatcgcgctgatccagatccagaacccgaaaaacctgaccgcaattaagatggcggattctgatgcgctgcgcgtgggtgattacaccgtagcaattggtaacccgttcggcctgggcgagacagtaacttccgggattgtctctgcgctgggacgtagcggcctgaacgctgaaaactacgaaaacttcatccagaccgatgcagcgatcaaccggggtaactcaggtggtgcgttggttaacctgaacggcgaactgatcggtatcaacaccgcgatcctcgcaccggacggcggaaacatcggtatcggttttgctatcccgagcaacatggtgaaaaacctgacctcgcagatggtggaatacggccaggtgaaacgtggtgagttgggtattatgggcactgagctgaactccgatctggcgaaagcgatgaaagttgacgcccagcgcggtgctttcgtaagccaggttctgccgaattcttccgccgcgaaagcgggcattaaagcgggtgatgtgatcacctcactgaacggtaagccaatcagcagctttgccgcactgcgtgctcaggtgggcactatgccggtaggtagcaaactgaccctgggcttactgcgcgacgggaagcaggttaacgtgaacctggaacttcagcagagcagccagaatcaggttgattccagcaccatcttcaacggcattgaaggcgctgagatgagcaacaaaggtaaagatcagggcgtggtggtgaacaacgtgaaaacgggcactccggctgcgcagatcggcctgaagaaaggtgatgtgattattggcgcgaaccagcaggcagtgaaaaacatcgctgaactgcgtaaagttctcgacagcaaaccgtctgtgctggcactgaacattcagcgcggcgacagcaccatctacctgttaatgcagtaa

SEQ ID NO: 86 - protein, 474 aa, Escherichia coli
MKKTTLALSALALSLGLALSPLSATAAETSSATTAQQMPSLAPMLEKVMPSVVSINVEGSTTVNTPRMPRNFQQFFGDDSPFCQEGSPFQSSPFCQGGLGGNGGGQQQKFMALGSGVIIDADKGYVVTNNHVVDNATVIKVQLSDGRKFDAKMVGKDPRSDIALIQIQNPKNLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVKNLTSQMVEYGQVKRGELGIMGTELNSDLAKAMKVDAQRGAFVSQVLPNSSAAKAGIKAGDVITSLNGKPISSFAALRAQVGTMPVGSKLTLGLLRDGKQVNVNLELQQSSQNQVDSSTIFNGIEGAEMSNKGKDQGVVVNNVKTGTPAAQIGLKKGDVIIGANQQAVKNIAELRKVLDSKPSVLALNIQRGDSTIYLLMQ

SEQ ID NO: 87 - DNA, 1476 bp, Shigella sonnei
atgaaaaaaaccacattagcactgagtcgactggctctgagtttaggtttggcgttatctccgctctctgcaacggcggctgagacttcttcagcaacgacagcccagcagatgccaagccttgcaccgatgctcgaaaaggtgatgccttcagtggtcagcattaacgtagaaggtagcacaaccgttaatacgccgcgtatgccgcgtaatttccagcagttcttcggtgatgattctccgttctgccaggaaggttcaccgtttcagagctctccgttctgccaggggggccagggcggtaatggcggcggccagcaacagaaattcatggcgctgggttccggcgtcatcattgatgccgataaaggctatgtcgtcaccaacaaccatgttgttgataacgcgacggtgattaaagtccagctgagcgatggccgtaagttcgacgcgaagatggttggcaaagatccgcgctctgatatcgcgctgatccaaatccagaacccgaaaaacctgaccgcaattaagatggcggattctgatgcactgcgcgtgggtgattacaccgtagcgattggtaacccgtttggtctgggcgagacggtaacttccgggattgtctctgcgctggggcgtagcggcctgaatgccgaaaactacgaaaacttcatccagaccgatgcagcgatcaaccgtggtaactccggtggtgcgctggttaacctgaacggcgaactgatcggtatcaacaccgcgatcctcgcaccggacggcggcaacatcggtatcggttttgctatcccgagtaacatggtgataaacctgacctcgcagatggtggaatacggccaggtgaaacgcggtgagctggggattatggggactgagctgatctccgaactggccaaagcgatgaaagttgacgcccagcgcggtgctttcgtaagccaggttctgcctaattcctctgctgcaaaagcgggcattaaagcgggtgatgtgatcacctcactgaacggtaagccgatcagcagctttgccgcactgcgtgctcaggtgggtactatgccggtgggcagcaaactgaccctgggcttactgcgcgacggtaagcaggttaacgtgaacctggaactgcagcagagcagccagaatcaggttgattccagctccatcttcaacggcattgaaggcgctgagatgatcaacaaaggcagagatcagggcgtggtagtgaacaacgtgaaaacgggcactccgcgtgcgcagatcggcctgaagaaaggtgatgtgattattggcgcgaaccagcaggcagtgaaaaacatcgctgaactgcgtaaagtgctcgacagcaaaccgtctgtgctggcactcaacattcagcgcggcgaccgccatctacctgttaatgcagtaatctccctcaaccccttcctgaaaacgggaaggggttctccttacaatctgtga

SEQ ID NO: 88 - protein, 491 aa, Shigella sonnei
MKKTTLALSRLALSLGLALSPLSATAAETSSATTAQQMPSLAPMLEKVMPSVVSINVEGSTTVNTPRMPRNFQQFFGDDSPFCQEGSPFQSSPFCQGGQGGNGGGQQQKFMALGSGVIIDADKGYVVTNNHVVDNATVIKVQLSDGRKFDAKMVGKDPRSDIALIQIQNPKNLTAIKMADSDALRVGDYTVAIGNPFGLGETVTSGIVSALGRSGLNAENYENFIQTDAAINRGNSGGALVNLNGELIGINTAILAPDGGNIGIGFAIPSNMVINLTSQMVEYGQVKRGELGIMGTELISELAKAMKVDAQRGAFVSQVLPNSSAAKAGIKAGDVITSLNGKPISSFAALRAQVGTMPVGSKLTLGLLRDGKQVNVNLELQQSSQNQVDSSSIFNGIEGAEMINKGRDQGVVVNNVKTGTPRAQIGLKKGDVIIGANQQAVKNIAELRKVLDSKPSVLALNIQRGDRHLPVNAVISLNPFLKTGRGSPYNL

SEQ ID NO: 89 - DNA, 1428 bp, Salmonella typhimurium
atgaaaaaaaccacattagcaatgagtgcactggctctgagtttaggtttggcattgtcgcctctgtctgccacggcggctgaaacgtcctcttcagcaatgactgcccagcagatgccaagcctggcaccgatgctcgaaaaagtgatgccatcggtggtcagtattaatgttgaaggtagcaccacggtgaatacgccgcgtatgccgcgtaatttccagcagttctttggcgatgactccccgttctgccaggacggttctccgttccagaattctccgttctgccagggcggcggtaacggcggcaacggcggtcaacaacagaaattcatggcgctgggctccggcgtaattattgacgccgcgaagggctacgtcgtcaccaacaaccacgtggttgataacgccagcgtgattaaagtacagcttagcgatgggcgtaaattcgatgctaaagtggtgggcaaagatccgcgttctgatatcgcgctgattcaaattcagaatccgaagaacctgacggcgattaagctggcggactccgacgcgctgcgcgtgggggattataccgtcgctattggtaacccgtttggtctgggcgaaacggtgacgtcaggtatcgtttcggcgctggggcgtagcggcctgaacgtagaaaattacgagaactttattcagaccgacgccgcgattaaccgtggtaactccggcggcgcgctggtgaacctgaacggtgagctgatcggtattaacaccgcgattctggcgccggacggcggcaacatcggtatcggcttcgctatccccagtaacatggtgaaaaacctgacgtcgcagatggtggaatacggccaggtgaaacgcggcgaactggggatcatggggactgagctgaattccgaattggcgaaagcgatgaaagtcgacgcccagcgaggcgcgttcgtcagccaggtgatgccgaattcgtccgcggcgaaagcgggtatcaaagccggggatgtcattacctcgctgaacggtaaaccgatcagcagctttgcggcgctgcgcgctcaggtcggcactatgccggtcggcagcaaaatcagcctcggtctgctgcgtgaaggtaaagcgattacggtgaatctggaactgcagcagagcagccagagtcaggttgattccagcaccatcttcagcgggattgaaggcgctgaaatgagcaataaaggccaggataaaggcgttgtggtgagcagcgtgaaagcgaactcacccgccgcgcaaattggcctcaaaaaaggcgatgtgattatcggcgctaaccagcagccggtgaaaaatatcgccgagctgcgtaagattctcgacagcaagccgtcggttctggcgctgaatattcagcgtggtgatagttctatttatttgctgatgcagtaa

SEQ ID NO: 90 - protein, 475 aa, Salmonella typhimurium
MKKTTLAMSALALSLGLALSPLSATAAETSSSAMTAQQMPSLAPMLEKVMPSVVSINVEGSTTVNTPRMPRNFQQFFGDDSPFCQDGSPFQNSPFCQGGGNGGNGGQQQKFMALGSGVIIDAAKGYVVTNNHVVDNASVIKVQLSDGRKFDAKVVGKDPRSDIALIQIQNPKNLTAIKLADSDALRVGDYTVAIGNPFGLGETVTSGIVSALGRSGLNVENYENFIQTDAAINRGNSGGALVNLNGELIGINTAILAPDGGNIGIGFAIPSNMVKNLTSQMVEYGQVKRGELGIMGTELNSELAKAMKVDAQRGAFVSQVMPNSSAAKAGIKAGDVITSLNGKPISSFAALRAQVGTMPVGSKISLGLLREGKAITVNLELQQSSQSQVDSSTIFSGIEGAEMSNKGQDKGVVVSSVKANSPAAQIGLKKGDVIIGANQQPVKNIAELRKILDSKPSVLALNIQRGDSSIYLLMQ

SEQ ID NO: 91 - DNA, 1428 bp, Salmonella enterica
atgaaaaaaaccacattagcaatgagtgcactggctctgagtttaggtttggcattgtcgcctctgtctgccacggcggctgaaacgtcctcttcagcaatgactgcccagcagatgccaagcctggcaccgatgctcgaaaaagtgatgccatcggtggtcagtattaatgtagaaggtagcaccacggtgaatacgccgcgtatgccgcgtaatttccagcaattctttggcgatgactccccgttctgccaggacggttctccgttccagaattctccgttctgccagggcggcggtaacggcggcaacggcggtcaacaacagaaattcatggcgctgggctccggcgtaattattgacgccgcgaagggctacgtcgtcaccaacaaccacgtggttgataacgccagcgtgattaaagtacagcttagcgatgggcgtaaattcgatgctaaagtggtgggcaaagatccgcgttctgatatcgcgctgattcaaattcagaatccgaagaacctgacggcgattaagctggcggactccgacgcgctgcgcgtgggggattataccgtcgctattggtaacccgtttggtctgggcgaaacggtgacgtcaggtatcgtttcggcgctggggcgtagcggcctgaacgtagaaaattacgagaactttattcagaccgacgccgcgattaaccgcggtaactccggcggcgcgctggtgaacctgaacggtgagctgatcggtattaacaccgcgattctggcgccggacggcggcaacatcggtatcggcttcgctatccccagtaacatggtgaaaaacctgacgtcgcagatggtggaatacggccaggtgaaacgcggcgaactggggatcatggggactgagctgagctctgagctggcgaaagcgatgaaagtcgacgcccagcgtggcgcgttcgtcagccaggtgatgccgaattcgtccgcagcgaaagcgggtatcaaagccggggatgtcattacctcgctgaacggtaaaccgatcagcagctttgcggcgctgcgcgctcaggtcggcactatgccggtcggcagcaaaatcagcctcggtctgctgcgtgaaggtaaagcgattacggttaatctggaactgcagcagagcagccagagtcaggttgattccagcaccatcttcagcgggattgagggcgctgaaatgagtaataaaggccaggataaaggcgttgtggtgagcagcgtgaaagcgaactcacccgccgcgcaaattggcctcaaaaaaggcgatgtgattatcggcgctaaccagcagccggtgaaaaatatcgccgagctgcgtaagattctcgacagcaagccgtcggtgctggcgctgaatattcagcgtggtgatagttctatttatttgctgatgcagtaa

SEQ ID NO: 92 - protein, 475 aa, Salmonella enterica
MKKTTLAMSALALSLGLALSPLSATAAETSSSAMTAQQMPSLAPMLEKVMPSVVSINVEGSTTVNTPRMPRNFQQFFGDDSPFCQDGSPFQNSPFCQGGGNGGNGGQQQKFMALGSGVIIDAAKGYVVTNNHVVDNASVIKVQLSDGRKFDAKVVGKDPRSDIALIQIQNPKNLTAIKLADSDALRVGDYTVAIGNPFGLGETVTSGIVSALGRSGLNVENYENFIQTDAAINRGNSGGALVNLNGELIGINTAILAPDGGNIGIGFAIPSNMVKNLTSQMVEYGQVKRGELGIMGTELSSELAKAMKVDAQRGAFVSQVMPNSSAAKAGIKAGDVITSLNGKPISSFAALRAQVGTMPVGSKISLGLLREGKAITVNLELQQSSQSQVDSSTIFSGIEGAEMSNKGQDKGVVVSSVKANSPAAQIGLKKGDVIIGANQQPVKNIAELRKILDSKPSVLALNIQRGDSSIYLLMQ

SEQ ID NO: 93 - DNA, 1434 bp, Klebsiella pneumoniae
atgaaaaaaaccacgttagcaatgagtgcactggctctgagtttaggtctggcgttgtccccgctctctgcgagcgcggcagaaacggcatcttcggccactaatgcacagcagatgccaagcctggccccgatgctggagaaggtgatgccgtcggtggtgagcattaacgttgaaggtagcaccaccgttaataccccacggatgccacgtaacttccagcagttctttggcgataattcgccgttctgccaggatggttctcctttccagagttcgccgttttgccagggcggcggacagggcggtcagcctgacggcggccagcaacagaaatttatggcgctgggctccggcgtcatcattgatgccgccaaagggtatgtggtgaccaacaaccacgtcgttgacaacgccaccacgattaaggttcagctgagcgatggccgtaggtttgatgccaaagtcgtcggcaaagatccgcgctctgatatcgcgctgattcagattcaggatccaaaaaacctgacggcgattaagcttgccgactccgatgcgctgcgcgtcggtgattacaccgtggctatcggcaacccgttcggtctgggcgaaaccgtgacctccgggattgtctccgcgctgggccgtagcggcctcaacgtggaaaactacgaaaactttatccagaccgatgcggcgattaaccgcgggaactccggcggtgcgctggtgaacctcaacggcgagctgatcggtatcaacaccgccattctggcgccggacggcggcaacatcggtatcggttttgctatcccgagcaacatggtgaaaaacctgaccgagcagatggtgaaatatggccaggtgaaacgcggtgagctcggcattatgggcaccgaactgaactctgagctggcgaaagcgatgaaagtcgatgcccagcgcggggccttcgtcagccaggtaatgccaggttcggcggccgcgaaagcgggcattaaagccggcgatgtcatcacgtccctcaacgggaaagcgatcagcagcttcgccgcgctgcgtgcccaggtcggcaccatgcctatcggcagcaaagttgagcttggtctgttgcgcgacggcaaaccggtgaccgtgaccgtcgaactgcagcagagcaaccagacccaggttgactccagcaccatcttcaacgggattgaaggggcagaaatgagcaataagggtcaggataagggcgtggtggtcaataatgtgaaagccggtaccccggcggcacagattggcctcaagaaaggcgatgtcatcattggggcgaatcagcagccggtgaaaaacatcgccgatctgtggaaaatcttcgacgccaaaccgtccgtactggcgctgaacattcagcgcggcgacgcctctatctatctgctgttgcagtaa

SEQ ID NO: 94 - protein, 477 aa, Klebsiella pneumoniae
MKKTTLAMSALALSLGLALSPLSASAAETASSATNAQQMPSLAPMLEKVMPSVVSINVEGSTTVNTPRMPRNFQQFFGDNSPFCQDGSPFQSSPFCQGGGQGGQPDGGQQQKFMALGSGVIIDAAKGYVVTNNHVVDNATTIKVQLSDGRRFDAKVVGKDPRSDIALIQIQDPKNLTAIKLADSDALRVGDYTVAIGNPFGLGETVTSGIVSALGRSGLNVENYENFIQTDAAINRGNSGGALVNLNGELIGINTAILAPDGGNIGIGFAIPSNMVKNLTEQMVKYGQVKRGELGIMGTELNSELAKAMKVDAQRGAFVSQVMPGSAAAKAGIKAGDVITSLNGKAISSFAALRAQVGTMPIGSKVELGLLRDGKPVTVTVELQQSNQTQVDSSTIFNGIEGAEMSNKGQDKGVVVNNVKAGTPAAQIGLKKGDVIIGANQQPVKNIADLWKIFDAKPSVLALNIQRGDASIYLLLQ

SEQ ID NO: 95 - DNA, 1416 bp, Edwardsiella tarda
atgaaaaaaaccactttagtattgagtgccgttgcattgagtctgggtatggcgttgagtccgatgtcggccagcgcggcagagacctcttccgccaccacagcccaggcgctgccgagcctggcaccaatgctggagaaagtgatgccgtccgtggtcagcatcaacgtcgagggcagcgcgaccgtcaataacagccgcgtgccgccgcagttccagcagttttttggcgaagactccccgctgtgccagccgggttcgccgttcggggattcgccgatgtgccagggcggcggcaatgcgccgtccaaagagcgctttaaggcgctgggctccggggtcattatcgacgcgggtaaaggctatgtcgtgaccaacaaccacgtggtggccaacgccgacagcattaaagtgcagctgagcgacggtcgccgcttcgacgccaaggtgatcggcaccgatccgcgtaccgatatcgcgctgatccagctgaaggatgccaagaacctgaccgccattaaaatggcggactctgacaatttgcgcgtgggcgactatgcggtggcgatcggcaacccctacgggctgggtgagaccgtcacctccgggatcgtttccgcgctgggccgcagcggcctgaacgtggaaaactacgaaaactttattcagactgacgcggcgattaaccgcggtaactccggcggtgcgctggtgaacctgaacggcgatctcatcgggatcaacaccgccattctggcgccggacggcggcaacatcggcatcggcttcgccattccgagcaacatggtgaaaaacttgaccggtcagatggtggagtacggccaggtgaaacgcggtgagctggggatcatggggaccgagctgaactccgagctggcgaaggcgatgaagatcgacgcccagcgcggcgcctttatcagccaggtgctgccgaagtcagccgccgcgaaggccgggctgaaacggctagatgtcatcgtctccctgaacgggaagaacatctccagcttcgcttccttccgcgctgaaatcggcaccatgccggtcggcagcaagctggcgctggggatcatccgcgacggtaagccgatgaacgtggaggtaaccttggagcagagcgcgcagagccaggtggcctctgacaacatcttcagcggtatcgaaggggccgagctgagcaatgcggccggtggcaagggcgtgaaggtggacaacgtgaagaaaggctccaccgccgcgcgtatcggtctgcagaagggcgacgtgatcctgggcgtgaaccagcacgcgatcgctaacatcggtgagctgcgtaaaattctggatgccaagccgccggtgctggcgctgaacatccagcgtggcgacaacaccctgtatctgctgatgcagtaa

SEQ ID NO: 96 - protein, 471 aa, Edwardsiella tarda
MKKTTLVLSAVALSLGMALSPMSASAAETSSATTAQALPSLAPMLEKVMPSVVSINVEGSATVNNSRVPPQFQQFFGEDSPLCQPGSPFGDSPMCQGGGNAPSKERFKALGSGVIIDAGKGYVVTNNHVVANADSIKVQLSDGRRFDAKVIGTDPRTDIALIQLKDAKNLTAIKMADSDNLRVGDYAVAIGNPYGLGETVTSGIVSALGRSGLNVENYENFIQTDAAINRGNSGGALVNLNGDLIGINTAILAPDGGNIGIGFAIPSNMVKNLTGQMVEYGQVKRGELGIMGTELNSELAKAMKIDAQRGAFISQVLPKSAAAKAGLKRLDVIVSLNGKNISSFASFRAEIGTMPVGSKLALGIIRDGKPMNVEVTLEQSAQSQVASDNIFSGIEGAELSNAAGGKGVKVDNVKKGSTAARIGLQKGDVILGVNQHAIANIGELRKILDAKPPVLALNIQRGDNTLYLLMQ

SEQ ID NO: 97 - DNA, 1437 bp, Yersinia enterocolitica
atgaaaaaaacaactttagtgttaagtgcattggcattgagcatcggtttagccatggggccggtttcttctgtcgttgcggcagaaacggcctcttccagcagccagcagctccctagcctggcgccaatgctagagaaggtaatgccttcggttgtgagtatcaatgtggagggaagtgctcaggtaagtaatgccggcggcataccaccacagttccagcaattctttggtgatgattcgccattctgccaggacggttctccgttccaagggtcaccaatgtgtcagggggggcctggtagcagggggggagcacccagcaagcaggatttccgtgcgctgggttctggcgtaattattgatgctgctaaaggttatgtcgtcactaataaccacgtagtagataatgcaaccaaaatcaatgttaagctcagtgatggccgtagttatgaagcaaaagtgattggtaaagatccgcgtactgatattgcattattgcagctgaaagacgctaaaaatctgactgcgattaaaattgccgactccgatcaactgcgcgttggggattataccgtagctatcggtaacccatacggcctgggtgaaaccgtcacttccggtatcgtgtcggcgttgggccgcagtggtttgaatgtggaaaactacgaaaactttatccagactgacgcggcaattaaccgtggtaactccggtggtgcattgatcaatctgaacggtgaactgatcgggatcaacacggctattctggcacctgatggcggcaacatcggtatcggttttgctatcccaagtaacatggtgaaaaacctgacctcacagatggttgaatttggtcaggtgaaacgcggtgaactgggcattatgggtacagagctgaactctgagctggcgaaagcaatgaaggttgatgcgcagaaaggggcctttatcagtcaggtactgccaaaatctgctgcagctaaagcagggattaaagcgggtgatgtgattgtcagcatgaatggcaaagccatcaatagcttcgctggcttccgtgccgagattggtactttgcctgtgggcagcaaaatgaccttgggtctgttgcgcgacggtaagcctgtcaacgtggaagtgacattagagcaaagcagccaaactcaagttgattcaggcaatctctacactggtattgaaggggctgagctgagcaactctgatgtgaatggtaagaaaggtgtgaaagttgatagtgtgaaacctggcactgctgctgcgcgtataggcctgaaaaaaggcgatgtcatcatgggtgtcaaccagcaacccgttcagaatctgggtgagctgcggaaaatccttgataccaaaccatcggtattggcattgagcattcagcgtggtgatacttcactctatctgctgatgcagtaa

SEQ ID NO: 98 - protein, 478 aa, Yersinia enterocolitica
MKKTTLVLSALALSIGLAMGPVSSVVAAETASSSSQQLPSLAPMLEKVMPSVVSINVEGSAQVSNAGGIPPQFQQFFGDDSPFCQDGSPFQGSPMCQGGPGSRGGAPSKQDFRALGSGVIIDAAKGYVVTNNHVVDNATKINVKLSDGRSYEAKVIGKDPRTDIALLQLKDAKNLTAIKIADSDQLRVGDYTVAIGNPYGLGETVTSGIVSALGRSGLNVENYENFIQTDAAINRGNSGGALINLNGELIGINTAILAPDGGNIGIGFAIPSNMVKNLTSQMVEFGQVKRGELGIMGTELNSELAKAMKVDAQKGAFISQVLPKSAAAKAGIKAGDVIVSMNGKAINSFAGFRAEIGTLPVGSKMTLGLLRDGKPVNVEVTLEQSSQTQVDSGNLYTGIEGAELSNSDVNGKKGVKVDSVKPGTAAARIGLKKGDVIMGVNQQPVQNLGELRKILDTKPSVLALSIQRGDTSLYLLMQ

SEQ ID NO: 99 - DNA, 1446 bp, Yersinia pseudotuberculosis
atgaaaaaaacaactttagtattaagtgcattggcattgagcattggtttcgccatgggcccggtttcttccgtcgttgcggcagagacggcagcatcgagtagccagcagctccctagcctggcgccaatgctagagaaagtaatgccttcagtggtcagtatcaacgttgaaggtagtgcgcctgtaagcagtgctggtgcacgcggtatgccaccacaattccagcagttttttggtgataactcgccattctgtcaggacggttcaccgttccaaggctcgccaatgtgtcaaggggatctgggcggactagggcagggaatgccaagtaagcgggaattccgttcgcttggttcaggtgtcattattgatgcgggcaaggggtatgtcgttaccaataaccacgtggtcgataatgcgaacaagatcagcgtaaaactgagcgatggccgcagttttgatgccaaggtgatcggtaaagatccacgtaccgatatcgcactgttacaactgaaagacgctaaaaatctgactgcgattaagattgccaattcggatcaactgcgtgtcggtgattataccgtcgctatcgggaacccgtatggcttgggtgaaaccgtgacatccggtattgtctctgctttagggcgcagtggtttgaatgtagaaaactatgaaaactttatccagactgatgcggcgattaaccgcggtaattccggcggcgcattaatcaacctgaacggtgagttgattggtattaacaccgctattctggcaccggatggcggtaacattggtattggctttgctatcccaagcaacatggtgaagaacctgacatcacagatggttgagtttggtcaggtaaaacgcggtgaactgggcattatggggaccgagctaaactctgaactggcaaaagccatgaaggttgatgcgcagaaaggtgcctttatcagccaggtcgtgcctaaatctgctgcggcaaaagcgggtatcaaagcgggcgatatcattgtcagtatgaatgggaaagccatcaatagttttgcagggttccgcgccgagatcggcacgttacctgttggcagcaaaatgaccttgggtctgctgcgtgatggcaaaccgatcaatgtgaatgtcgtcctggagcagagcagccacagtcaggtggaatccggtaatctctacaccggtattgagggggctgaactgagtaacagcaacgttagcggcaagaaaggggtgaaagttgatagcgtaaaaccaggcactgctgcggcgcgtatcggcctgaaaaaaggtgatatcataatggggattaaccagcaaccagtccagaacctaggtgagctgcggaaaatcctcgatgctaaaccaccggtattggcgttgaatattcaacgtggtgatacttcactctatttattgatgcagtaa

SEQ ID NO: 100 - protein, 481 aa, Yersinia pseudotuberculosis
MKKTTLVLSALALSIGFAMGPVSSVVAAETAASSSQQLPSLAPMLEKVMPSVVSINVEGSAPVSSAGARGMPPQFQQFFGDNSPFCQDGSPFQGSPMCQGDLGGLGQGMPSKREFRSLGSGVIIDAGKGYVVTNNHVVDNANKISVKLSDGRSFDAKVIGKDPRTDIALLQLKDAKNLTAIKIANSDQLRVGDYTVAIGNPYGLGETVTSGIVSALGRSGLNVENYENFIQTDAAINRGNSGGALINLNGELIGINTAILAPDGGNIGIGFAIPSNMVKNLTSQMVEFGQVKRGELGIMGTELNSELAKAMKVDAQKGAFISQVVPKSAAAKAGIKAGDIIVSMNGKAINSFAGFRAEIGTLPVGSKMTLGLLRDGKPINVNVVLEQSSHSQVESGNLYTGIEGAELSNSNVSGKKGVKVDSVKPGTAAARIGLKKGDIIMGINQQPVQNLGELRKILDAKPPVLALNIQRGDTSLYLLMQ

SEQ ID NO: 101 - DNA, 1446 bp, Yersinia pestis
atgaaaaaaacaactttagtattaagtgcattggcattgagcattggtttcgccatgggcccggtttcttccgtcgttgcggcagagacggcagcatcgagtagccagcagctccctagcctggcgccaatgctagagaaagtaatgccttcagtggtcagtatcaacgttgaaggtagtgcgcctgtaagcagtgctggtgcacgcggtatgccaccacaattccagcagttttttggtgataactcgccattctgtcaggacggttcaccgttccaaggctcgccaatgtgtcaaggggatctgggcggactagggcagggaatgccaagtaagcgggaattccgttcgcttggttcaggtgtcattattgatgcgggcaaggggtatgtcgttaccaataaccacgtggtcgataatgcgaacaagatcagcgtaaaactgagcgatggccgcagttttgatgccaaggtgatcggtaaagatccacgtaccgatatcgcactgttacaactgaaagacgctaaaaatctgactgcgattaagattgccaattcggatcaactgcgtgtcggtgattataccgtcgctatcgggaacccgtatggcttgggtgaaaccgtgacatccggtattgtctctgctttagggcgcagtggtttgaatgtagaaaactatgaaaactttatccagactgatgcggcgattaaccgtggtaattccggcggcgcattaatcaacctgaacggtgagttgattggtattaacaccgctattctggcaccggatggcggtaacattggtattggctttgctatcccaagcaacatggtgaagaacctgacatcacagatggttgagtttggtcaggtaaaacgcggtgaactgggcattatggggaccgagctaaactctgaactggcaaaagccatgaaggttgatgcgcagaaaggtgcctttatcagccaggtcgtgcctaaatctgctgcggcaaaagcgggtatcaaagcgggcgatatcattgtcagtatgaatgggaaagccatcaatagttttgcagggttccgcgccgagatcggcacgttacctgttggcagcaaaatgaccttgggtctgctgcgtgatggcaaaccgatcaatgtggatgtcgtcctggagcagagcagccacagtcaggtggaatccggcaatctctacaccggtattgagggggctgaactgagtaacagcgacgttagcggcaagaaaggggtgaaagttgatagcgtaaaaccaggcactgctgcggcgcgtatcggcctgaaaaaaggtgatatcatcatggggattaaccagcaaccagtccagaacctaggtgagctgcggaaaatcctcgatgctaaaccaccggtattggcgttgaatattcaacgtggtgatacttcactctatttattgatgcagtaa

SEQ ID NO: 102 - protein, 481 aa, Yersinia pestis
MKKTTLVLSALALSIGFAMGPVSSVVAAETAASSSQQLPSLAPMLEKVMPSVVSINVEGSAPVSSAGARGMPPQFQQFFGDNSPFCQDGSPFQGSPMCQGDLGGLGQGMPSKREFRSLGSGVIIDAGKGYVVTNNHVVDNANKISVKLSDGRSFDAKVIGKDPRTDIALLQLKDAKNLTAIKIANSDQLRVGDYTVAIGNPYGLGETVTSGIVSALGRSGLNVENYENFIQTDAAINRGNSGGALINLNGELIGINTAILAPDGGNIGIGFAIPSNMVKNLTSQMVEFGQVKRGELGIMGTELNSELAKAMKVDAQKGAFISQVVPKSAAAKAGIKAGDIIVSMNGKAINSFAGFRAEIGTLPVGSKMTLGLLRDGKPINVDVVLEQSSHSQVESGNLYTGIEGAELSNSDVSGKKGVKVDSVKPGTAAARIGLKKGDIIMGINQQPVQNLGELRKILDAKPPVLALNIQRGDTSLYLLMQ

SEQ ID NO: 103 - DNA, 1467 bp, Erwinia carotovora
atgaaaagaaaatcactggttctgagtgcgctggcgttaagtctggcgatggcgatgggctccactacggcaaatgcggctgagtcagcggcatctgctgcgtcatcaggtcaattacctagtctggcccctatgctggaaaatgttatgccttccgtggtgagcatctatgtggaagggcataccaccaatgcgggtaaagaaagcaatgcaggcaaagaaggtataccgccgcagcttcagccgttttttggtgaaaactcgccgttctgccaagagggatcgccgttccagtcgtcgccgatgtgtcagggcgatagtgacgacgatgacagcggccaaccgcaaccaaaacaggaaaacttccaggcgctgggcgcgggcgtagtgattaatgcggaaaaaggctacgtggtgaccaatagtcacgtggtggataacgccgataaaattcagattcgactcagtgatggccgcaagtatgacggtaaagtgctaggcaaagacccgcgttcagatatcgcgctggtgcagttgaaggactttaaaaatctgacggccattaaggttgcggattccgaccaactgcgggtcggtgattacaccgtagcgattggtaacccatatggcctgggggaaaccgcgacatcgggcattgtgtccgcgctgggacgcagcggcttgaatattgaaaactacgagaactttattcagaccgatgcggcgattaatcggggtaattccggcggggcgctggtgaacctgaatggggaattgattgggctgaataccgcgattcttgcgccagacggcggcaatatcgggatcggtttcgctatccccagcaatatggtgaaaagtgtcgtcgcacagattatcgaatttggcgaagtgaagcgtggcgaactgggcatcacgggtacggagttgaattccgaactggcgcaggcgatgaaggttgatgcacagcgcggcgcgtttgtgagtcaggtgcggccgaaatcggcggcagatgcggcgggtatcaaagcgggcgatgtgatcgtcacgctgaatggcaaagcggttagcagtttctccgcgctgcgtgcgcaggtcggttcgctgccggtgggcagcaaagtcgcactggggctgctgcgtgatggcaaaccgctgacggttgatgtgacgctgcaacagagcaatcaggctcaggtggcttccggtaatctctactccggcattgaaggtgctgaactgagtaatactcagattgacgataaaaaaggcgttaaggtggataacgttaaacccggttccgcggcggctaaagtggggctgaagaaggacgacattattctcggcgttaaccaacaggtggttcagaatatcggcgaattgcgtaaaattctggacagcaaaccggcagtcttggcgttgaacgttcgtcgaggtgacagcacgatttatctgctggctcagtaa

SEQ ID NO: 104 - protein, 488 aa, Erwinia carotovora
MKRKSLVLSALALSLAMAMGSTTANAAESAASAASSGQLPSLAPMLENVMPSVVSIYVEGHTTNAGKESNAGKEGIPPQLQPFFGENSPFCQEGSPFQSSPMCQGDSDDDDSGQPQPKQENFQALGAGVVINAEKGYVVTNSHVVDNADKIQIRLSDGRKYDGKVLGKDPRSDIALVQLKDFKNLTAIKVADSDQLRVGDYTVAIGNPYGLGETATSGIVSALGRSGLNIENYENFIQTDAAINRGNSGGALVNLNGELIGLNTAILAPDGGNIGIGFAIPSNMVKSVVAQIIEFGEVKRGELGITGTELNSELAQAMKVDAQRGAFVSQVRPKSAADAAGIKAGDVIVTLNGKAVSSFSALRAQVGSLPVGSKVALGLLRDGKPLTVDVTLQQSNQAQVASGNLYSGIEGAELSNTQIDDKKGVKVDNVKPGSAAAKVGLKKDDIILGVNQQVVQNIGELRKILDSKPAVLALNVRRGDSTIYLLAQ

SEQ ID NO: 105 - DNA, 1371 bp, Erwinia carotovora
atgaaaaaaacgtcattattatttagtgcactggcaatgagtataggtttgaccctgtccacgcttcccgcagcgaatgctgcgctaccttccgtggttcaagggcaacaaacgccaagcctggccccaatgctggaaaaagtcttaccagctgtcgtcagcgtgcatgttgaaggtacacaggtacagcgccagcgcgtaccggaagagttcaagttcttctttgggccaaatttcccgacggacaaacaaaattctcgtccgtttgaagggctgggttccggcgtgattattgatgcagcaaaagggtatgtgctcaccaacaatcacgttatcaataatgccgacaaaattcgcgtccagcttaatgacgggcgtgaatatgatgcgaaactgattggtcgcgacgagcagaccgatattgccctgctacagctgaatgacgccaaaaacctagtatccgtaaaaatggcggattccgatcagttgcgcgtcggtgattttgccgttgccgtgggtaacccattcggcctcggccagaccgcgacatccggcattatctctgcactgggacgtagcggcctgaaccttgaagggctggaaaacttcatccagaccgatgcttctatcaaccgcggtaactccggcggtgcgctggttaacctcaacggcgaactgatcggtatcaacaccgcgattctggctccgggcggaggaaacatcggtatcggtttcgccatccccagcaacatggctcagaatctggcgcagcagttggttgaatttggtgaagttaaacgcgggctgctgggtattaaaggcagcgagatgacgtctgagatggcgaaagccttcaacgtcgatgcacagcgcggcgctttcgtcagcgaagtcttaccgaaatctgccgcgtccaaagcgggtatcaaggctggcgacgtgttgactactctggatggtaaaccgatcagcagctttgcagaactgagagccaaagtcggcaccaccgcgccgggcaagaccgtgaaaatcggcctgctgcgtgatggtaaaccacaggaggtttcggtggtgttggataacagcacatcggcatcgaccagcgccgaaacgctttcaccgtcattgcagggtgcatcgctgatcaatggtcaattgaaagatggcagcaaaggcgtgcagattgataacgtcgctaaggacacgcctgctgcgcaggttggtctgcaaaaaggcgatatcatcattggcgtaaaccgcgagcgcattgaaaacatcacgcaactgcgcaagctgctggaagcgaagccttccgttctggcactgaacatcgtccgtggcgaagaaacgatctatctgctgctacgttaa

SEQ ID NO: 106 - protein, 456 aa, Erwinia carotovora
MKKTSLLFSALAMSIGLTLSTLPAANAALPSVVQGQQTPSLAPMLEKVLPAVVSVHVEGTQVQRQRVPEEFKFFFGPNFPTDKQNSRPFEGLGSGVIIDAAKGYVLTNNHVINNADKIRVQLNDGREYDAKLIGRDEQTDIALLQLNDAKNLVSVKMADSDQLRVGDFAVAVGNPFGLGQTATSGIISALGRSGLNLEGLENFIQTDASINRGNSGGALVNLNGELIGINTAILAPGGGNIGIGFAIPSNMAQNLAQQLVEFGEVKRGLLGIKGSEMTSEMAKAFNVDAQRGAFVSEVLPKSAASKAGIKAGDVLTTLDGKPISSFAELRAKVGTTAPGKTVKIGLLRDGKPQEVSVVLDNSTSASTSAETLSPSLQGASLINGQLKDGSKGVQIDNVAKDTPAAQVGLQKGDIIIGVNRERIENITQLRKLLEAKPSVLALNIVRGEETIYLLLR

SEQ ID NO: 107 - DNA, 1470 bp, Saccharomyces bayanus
atgtctgctgatttcggtttgattggtttggccgtcatgggtcaaaacttgatcttaaacgctgctgatcacggtttcactgtttgtgcttacaacagaactcaatccaaggttgaccatttcttggctaacgaagccaaaggtaagtctatcattggtgctacttccattcaagatttcatttccaagttgaagagacctagaaaggtcatgcttttggttaaagccggtgctccagtcgacgccttgatcaaggaaattgttccacttttggaaaagggcgacatcatcatcgatggtggtaactcccatttcccagactctaacagacgttacgaagaattgaagaagcacggtattcttttcgtcggttccggtgtctctggtggtgaagatggtgcccgttatggtccatctttgatgccaggtggttctgaagaagcttggccacacatcaagaacatcttccaatctatctccgcaaagtctgatggtgaaccatgttgtgaatgggtcggcccagctggtgccggtcactacgtcaaaatggtccacaacggtatcgaatatggtgacatgcaattgatttgtgaagcttacgatatcatgaagagattgggtggattcactgacaaagaaatcagtgaagttttcaccacatggaacaagggtgtccttgactctttcttgattgaaattaccagagatatcttgaaattcgacgatgtcgacggtaagccattggtggaaaaaatcatggatactgccggccaaaagggtaccggtaagtggactgccatcaacgccttggacttgggtatgccagtcaccttgatcggtgaagctgtctttgctcgttgtctatctgccttgaagaatgaaagagtcagagcttctaaggttttgccaggcccacaagttccaaaggacgccgtcaaggacagaaagcaatttgtcgatgatttggaacaagctttgtacgcttctaagatcatctcctacgctcaaggtttcatgttgatccgtgaagccgctgccacatacggatggaaactaaacaaccctgctattgctttgatgtggagaggtggttgtatcattagatctgttttcttggccgaaatcaccaaggcttatagacaagaaccagacctagaaaacttgttgttcaacaagttcttcgccgatgctgttaccaaggctcaatctggttggagaaaatccattgcattggctaccacatacggtatcccaaccccagccttttccactgctttgaccttctacgacgggtacagatctgaaagattaccagccaacttactacaagctcaacgtgattacttcggtgctcacactttcagagtcttgccagaatgtgcatccgaaaacttgccagaagacaaggatattcatatcaactggaccggtcatggtggtaacgtttcttcctcaacataccaagcttaa

SEQ ID NO: 108 - protein, 489 aa, Saccharomyces bayanus
MSADFGLIGLAVMGQNLILNAADHGFTVCAYNRTQSKVDHFLANEAKGKSIIGATSIQDFISKLKRPRKVMLLVKAGAPVDALIKEIVPLLEKGDIIIDGGNSHFPDSNRRYEELKKHGILFVGSGVSGGEDGARYGPSLMPGGSEEAWPHIKNIFQSISAKSDGEPCCEWVGPAGAGHYVKMVHNGIEYGDMQLICEAYDIMKRLGGFTDKEISEVFTTWNKGVLDSFLIEITRDILKFDDVDGKPLVEKIMDTAGQKGTGKWTAINALDLGMPVTLIGEAVFARCLSALKNERVRASKVLPGPQVPKDAVKDRKQFVDDLEQALYASKIISYAQGFMLIREAAATYGWKLNNPAIALMWRGGCIIRSVFLAEITKAYRQEPDLENLLFNKFFADAVTKAQSGWRKSIALATTYGIPTPAFSTALTFYDGYRSERLPANLLQAQRDYFGAHTFRVLPECASENLPEDKDIHINWTGHGGNVSSSTYQA

SEQ ID NO: 109 - DNA, 1470 bp, Saccharomyces castellii
atgtccgctgattttggtttaatcggtttggccgtcatgggtcaaaacttgatcttaaatgccgcagaccacggtttcaccgtctgtgcttacaacagaactgtctccaaagtggaacatttcctagctaacgaagccaagggtaagtccattgttggtgctacttctatcgacgactttttgtctaaattgaagagaccaagaaaggttatgcttcttgttaaggctggtgctccagttgatgctctaattaacgaattggtcccacgtctagaaaagggtgatattatcatcgatggtggtaactctcatttcccagacaccaacagacgttacgaagaattgaagcaaaagggtatcttgtttgttggttccggtgtctctggtggtgaagatggtgcccgttacggtccatctttgatgccaggtggtgcagaagaagcttggccacacattaaggatattttccaatccatctccgctaaatctgatggtgaaccatgttgtgactgggtcggtccagccggtgcaggtcattacgtcaaaatggtccataacggtattgaatacggtgacatgcaattgatttgtgaagcttatgacattatgaagcgtgtcggtcatttcactgataaggaaattggtgatgtttttgaaacctggaacaagggtgtcttggattctttcctaattgaaattaccagagatatcttgaaattcaatgacgttgatggtaagccattggttgaaaagattatggataccgctggtcaaaagggtactggtaagtggaccgctattaatgctctagatttgggtattccagtcactttgattggtgaagctgtcttctctcgttgtctatctgctttgaagtctgaaagaattagagcttccaagactctattgggtccagaagttccaaaggatttgattaaggataagaagcaatttgttgacgatttggaacaagccttatacgcttccaaaattatgtcctacgctcaaggtttcatgttaatccgtgaagctggtaagacttatggttggaaactaaacaacccagctattgccttgatgtggagaggtggttgtattattagatccgtcttcttgggtgaaattaccaaggcttacagagaagatccagaactagaaaatctattattcaacaagttcttctccgatgctgtcactaaggctcaaaccggttggagaaagtccattgctttggctaccacttacggtatcccaaccccagcattctccactgctttggccttctacgatggttacagatctgaaagattaccagctaacttgctacaagctcaaagagattacttcggtgctcacactttcagagtcttgccagaatgtgcctctgacaacttgccattagacaaggatatccacattaactggaccggtcacggtggtaacgtttcttcttctacttaccaagcttaa

SEQ ID NO: 110 - protein, 489 aa, Saccharomyces castellii
MSADFGLIGLAVMGQNLILNAADHGFTVCAYNRTVSKVEHFLANEAKGKSIVGATSIDDFLSKLKRPRKVMLLVKAGAPVDALINELVPRLEKGDIIIDGGNSHFPDTNRRYEELKQKGILFVGSGVSGGEDGARYGPSLMPGGAEEAWPHIKDIFQSISAKSDGEPCCDWVGPAGAGHYVKMVHNGIEYGDMQLICEAYDIMKRVGHFTDKEIGDVFETWNKGVLDSFLIEITRDILKFNDVDGKPLVEKIMDTAGQKGTGKWTAINALDLGIPVTLIGEAVFSRCLSALKSERIRASKTLLGPEVPKDLIKDKKQFVDDLEQALYASKIMSYAQGFMLIREAGKTYGWKLNNPAIALMWRGGCIIRSVFLGEITKAYREDPELENLLFNKFFSDAVTKAQTGWRKSIALATTYGIPTPAFSTALAFYDGYRSERLPANLLQAQRDYFGAHTFRVLPECASDNLPLDKDIHINWTGHGGNVSSSTYQA

SEQ ID NO: 111 - DNA, 1470 bp, Candida glabrata
atgtctgctgatttcggtttgattggtttggccgttatgggtcaaaacttgatcctaaacattgctgaccacggttacactgttgttgcttacaacagatccgttgacaaggtccaccaattcttggacaacgaagccaagggtaagtccatcatcggtgctgagtccatcgaggacttggtcgccaagttgaagagacctagaaagatcatgatgttggtcaaggccggtgccccagtcgacggtttgatcgctcaattggttccacacttggaaaagggtgacatcatcattgacggtggtaactcccacttcccagactctaacagacgttacgaggagttgaaggccaagggtatcttgttcgtcggctctggtgtctctggtggtgaagacggtgcccgttacggtccatctttgatgccaggtggtgccgaagaagcttggccacacatcaaggagatcttccaatccatctccgccaagtccgacggtgagccatgttgtgactgggttggcccagctggttctggtcactacgtcaagatggtccacaacggtatcgaatacggtgacatgcagttgatctgtgaagcctacgacatcatgaagagaatcggtggtttcaccgacaaggaaatctccgaagtcttcggcaagtgggacactggtgttctagactctttcttggtcgaaattaccaccaacatcctaaagaaggacgacgttgacggtaagccattggttgaaaagatcatggactctgctggtcaaaagggtaccggtaagtggaccgccatcaacgccttggacctaggtatgccagtctctttgattggtgaagccgtcttcgctagatgtttgtctgctctaaagtgtgaaagagttaaggcctccaagactttgccaggcccagaaattccaaaggacttgatcaaggacagacaacaattcattgacgacttggaacaagccctatacgcttctaagatcatctcctacgctcaaggtttcatgttgatcagagaagccgctaagacttacggctggaagctaaacaacccagctattgctctaatgtggagaggtggttgtatcatcagatccgtcttcttgggtgaaatcaccaaggcttacagagaaaacccagacttggaaaacttgctattccacaagttcttcaaggacgccgtcaccaaggctcaaggtggctggagaagatctttggctttggctgccacttacggtatcccatccccagccttcgccaccgctttgtccttctacgatggttacagatctgaaagattgccagctaacttgttgcaagctcaacgtgactacttcggtgctcacactttcagagtcttgccagaatgtgcttccgactccctaccaaaggacaaggacatccacatcaactggaccggtcacggtggtaacgtctcttctaccacttacgatgcttaa

SEQ ID NO: 112 - protein, 489 aa, Candida glabrata
MSADFGLIGLAVMGQNLILNIADHGYTVVAYNRSVDKVHQFLDNEAKGKSIIGAESIEDLVAKLKRPRKIMMLVKAGAPVDGLIAQLVPHLEKGDIIIDGGNSHFPDSNRRYEELKAKGILFVGSGVSGGEDGARYGPSLMPGGAEEAWPHIKEIFQSISAKSDGEPCCDWVGPAGSGHYVKMVHNGIEYGDMQLICEAYDIMKRIGGFTDKEISEVFGKWDTGVLDSFLVEITTNILKKDDVDGKPLVEKIMDSAGQKGTGKWTAINALDLGMPVSLIGEAVFARCLSALKCERVKASKTLPGPEIPKDLIKDRQQFIDDLEQALYASKIISYAQGFMLIREAAKTYGWKLNNPAIALMWRGGCIIRSVFLGEITKAYRENPDLENLLFHKFFKDAVTKAQGGWRRSLALAATYGIPSPAFATALSFYDGYRSERLPANLLQAQRDYFGAHTFRVLPECASDSLPKDKDIHINWTGHGGNVSSTTYDA

SEQ ID NO: 113 - DNA, 1482 bp, Saccharomyces servazzii
atgtctcaagaagctgtcgctgacttcggtctaattggtcttgccgtcatgggccaaaatttaatcctaaacgttgctgatcacggtttcaccgtctgtgcctacaacagaaccgtctctaaagtcgacgatttcttagccaacgaagccaagggcaaatccatcgtcggcgccaagtctatcgaagaattcatcgccaaattgaaaagaccaagaaaaatcatgctattagtcaaagccggtaaaccagtagactacatcatcaacgacctattaccacatctagaaaagggtgatatcatcatcgatggtggtaactcccatttcccagacaccaacagaagatacgaagaattgaccaagaagggcattcttttcgtcggctctggtgtctccggtggtgaagacggggcccgttacggtccttccttaatgccaggtggtgccgaagaagcttggcctcacatcaaggacatcttccaatccatctctgctaaagccgacggtgaaccttgttgtgactgggtcggccctgccggggccggtcactacgtcaagatggtccacaacggtatcgaatacggtgacatgcaattgatctgtgaagcctacgatatcatgaaacgtatcggtggcttctccgacaaagaaatctcagacgtcttcgccacctggaacaagggggtcctagattccttcttaatcgaaatcaccagagatatcctagccttcaacgacaaagatggtaaaccattagtcgaaaaaatcttagacagtgctggccaaaagggtaccggtaaatggaccgccatcaacgccctagacttaggtatgcctgtcacccttatcggtgaagccgtctttgctcgttgtctatccgctttaaagagtgaaagaatcttggcctccaaacaattagccggcccaaccatcccagccgatgccatcaaggacaagaaacaattcgtcgacgaattagaacaagccttatacgcctccaagatcatgtcctacgcccaagggttcatgttgatcagagaagccgccaagacctacggttggaaattaaataacccagctatcgctttaatgtggagaggtggttgtatcatcagatctgtcttcttaggtgaaattaccaaagcttatagagaaaacccagatttagaaaacttattattgaacaaattcttctcagatgccgtcaccaaggctcaaaccggttggagaaagaccatcggtttagctaccacctacggtatcccaaccccaaccttctctaccgctttagccttctacgatggttacagatctgaaagattaccagctaacttattacaagctcaaagagattacttcggtgctcacactttcagagtcttaccagaagaagcttgtgaaactttaccagaaggtaaggatatccacatcaactggaccggtcacggtggtaacgtctcttcttccacttatgatgcttag

SEQ ID NO: 114 - protein, 493 aa, Saccharomyces servazzii
MSQEAVADFGLIGLAVMGQNLILNVADHGFTVCAYNRTVSKVDDFLANEAKGKSIVGAKSIEEFIAKLKRPRKIMLLVKAGKPVDYIINDLLPHLEKGDIIIDGGNSHFPDTNRRYEELTKKGILFVGSGVSGGEDGARYGPSLMPGGAEEAWPHIKDIFQSISAKADGEPCCDWVGPAGAGHYVKMVHNGIEYGDMQLICEAYDIMKRIGGFSDKEISDVFATWNKGVLDSFLIEITRDILAFNDKDGKPLVEKILDSAGQKGTGKWTAINALDLGMPVTLIGEAVFARCLSALKSERILASKQLAGPTIPADAIKDKKQFVDELEQALYASKIMSYAQGFMLIREAAKTYGWKLNNPAIALMWRGGCIIRSVFLGEITKAYRENPDLENLLLNKFFSDAVTKAQTGWRKTIGLATTYGIPTPTFSTALAFYDGYRSERLPANLLQAQRDYFGAHTFRVLPEEACETLPEGKDIHINWTGHGGNVSSSTYDA

SEQ ID NO: 115 - DNA, 1479 bp, Kluyveromyces lactis
atgtctgaacctgccggtgatattggtttgattggtttggccgttatgggtcaaaacttgatcttgaacgctgctgaccacggtttcactgttgttgcttataacagaacagtttctaaggttgatcatttcttagcaaacgaggcaaagggtaaatctatcattggtgctcattctgtggaagaattgtgccgtaacttgaagagaccaagaagaatcattcttttggtcaaggctggtgatgctgtggatgctttcattcaacaattgttgccatttttggaaaagggagacattatcatcgatggtggtaactctcatttcccagattctaacagacgttacgatgaattgaaggaaaagggaatctactttgttggttctggtgtttctggtggtgaagaaggtgctcgttacggtccttctttgatgccaggtggtgccgaagaagcttggcctcacattaaggacatcttccaatctatttctgccaagtctgacggcgaaccatgttgtgactgggttggtccagctgggtctggtcattacgtgaagatggtccacaacggtatcgaatacggtgacatgcaattgatcactgaagcttacgatatcatgaagagaattggtggattcaccgataaggaaatcggtgaagttttctcccaatggaacaagggtgtcttggattccttcttggttgaaattacaagagacattttgctgtacgacgatgtcgatggtactcctctagtggaaaagatcttggattcagctggtcaaaagggtactggtaaatggactgccattaacgctttggatctaggtatgccagttactttgattggtgaagccgtgttcgctcgttgtttgtcctctttgaaggacgaaagagttagagcctccaagttgcttccaggccctcaaattccaaaggatgccgttaaggaaagacaacaattcgttgacgacttggaacaagctctttacgcttccaagatcatctcttacgctcaaggtttcatgttgatccgtgaagctggtaagacttacggctggaagttgaacaaccctgccatcgctttgatgtggagaggtggttgtatcatcagatccgttttcttgggtgaaattacaaaggcttatagagaaaacccagaattggaaaacttattattcaacaaattcttcgctgatgccgtcgaaaaggctcaaagcggttggagaaagactattggtttggctgtttcttacggtatcccaaccccagctttctccactgctctatcgttctacgatggttacagatctgaaagattgccagctaacttgctacaagctcaacgtgattacttcggtgctcacactttcagagtcttgccagaagctgcttccgaaaacttgccagctgaccaaaacatccacattaactggaccggtaaaggtggtaacgtttcagcttccagttaccaagcttaa

SEQ ID NO: 116 - protein, 492 aa, Kluyveromyces lactis
MSEPAGDIGLIGLAVMGQNLILNAADHGFTVVAYNRTVSKVDHFLANEAKGKSIIGAHSVEELCRNLKRPRRIILLVKAGDAVDAFIQQLLPFLEKGDIIIDGGNSHFPDSNRRYDELKEKGIYFVGSGVSGGEEGARYGPSLMPGGAEEAWPHIKDIFQSISAKSDGEPCCDWVGPAGSGHYVKMVHNGIEYGDMQLITEAYDIMKRIGGFTDKEIGEVFSQWNKGVLDSFLVEITRDILLYDDVDGTPLVEKILDSAGQKGTGKWTAINALDLGMPVTLIGEAVFARCLSSLKDERVRASKLLPGPQIPKDAVKERQQFVDDLEQALYASKIISYAQGFMLIREAGKTYGWKLNNPAIALMWRGGCIIRSVFLGEITKAYRENPELENLLFNKFFADAVEKAQSGWRKTIGLAVSYGIPTPAFSTALSFYDGYRSERLPANLLQAQRDYFGAHTFRVLPEAASENLPADQNIHINWTGKGGNVSASSYQA

SEQ ID NO: 117 - DNA, 1488 bp, Debaryomyces hansenii
atgtctgctccaactggtgatattggtttaattggtttagccgttatgggtcaaaacttgatcttaaacgctgctgacaatggttacactgtcgtagcttacaacagaactgttgcaaaggtcgacgacttcttggacaacgaggctaagggcaaatcagtcattggtgctcactccatcgaagaattatgtgctaacttgaagagaccaagaagaatcattttattagttaaggctggtaaaccagttgacgcttttattgaacaattattacctcacttagaaaagggtgatatcatcattgacggtggtaactcccatttcccagattctaaccgtcgttacgaagaattaaacggtaagggtatcttattcgttggttctggtgtttctggtggtgaagaaggtgctagatacggtccatctttgatgccaggtggtcacaaggacgcttggccacacatcaaggatatcttccaaagtattgctgccaagtctgatggtgaaccatgttgtgactgggttggtgatgccggtgctggtcattacgttaagatggtgcacaatggtattgaatacggtgacatgcaattgatttgtgaagcttatgatttattaaagagagttggtaaattctccaacaaggaaatcggtgacgttttcgctaaatggaacaagggtgttttagactctttcttaattgaaattaccagagatattttgtacttcaatgatccaactgacaataagccattattagaaaagattttggatactgctggtcagaagggtactggtaagtggactgctatcaatgctttagatttaggtatgcctgtcaccttaattggtgaagctgttttctctcgttgtttatccgccttgaaggatgaaagagtcagagcttctaaatcattagccggcccatcagttgaagatgacgaatctccaattcaagataagcaaaagttcgttgacgacttagaacaagctttatatgcttctaagattatctcttacgctcaaggtttcatgttgattagagaagctgctaaggaatacggctggaaattaaataacccagctatcgccttaatgtggagaggtggttgtattatcagatctgttttcttaggtgaaatcacttctgcttaccgtgaaaccccagacttagaaaacttattattccatccattcttcaatgatgccatcaaaaaggctcaaaagggttggagatcaactattggtaaggctgttgaatttggtgttccagttccagcttttagcactgctttatctttctacgatggttacagatctgctaagttaccagctaacttgttgcaagctcaaagagattacttcggtgctcacactttccaagtgttaccaggtgaagaaaatgatttcttaaagaaggaccaatggatccatgttaactggaccggtaagggtggtaatgtttccgcctctacttatgatgcttag

SEQ ID NO: 118 - protein, 495 aa, Debaryomyces hansenii
MSAPTGDIGLIGLAVMGQNLILNAADNGYTVVAYNRTVAKVDDFLDNEAKGKSVIGAHSIEELCANLKRPRRIILLVKAGKPVDAFIEQLLPHLEKGDIIIDGGNSHFPDSNRRYEELNGKGILFVGSGVSGGEEGARYGPSLMPGGHKDAWPHIKDIFQSIAAKSDGEPCCDWVGDAGAGHYVKMVHNGIEYGDMQLICEAYDLLKRVGKFSNKEIGDVFAKWNKGVLDSFLIEITRDILYFNDPTDNKPLLEKILDTAGQKGTGKWTAINALDLGMPVTLIGEAVFSRCLSALKDERVRASKSLAGPSVEDDESPIQDKQKFVDDLEQALYASKIISYAQGFMLIREAAKEYGWKLNNPAIALMWRGGCIIRSVFLGEITSAYRETPDLENLLFHPFFNDAIKKAQKGWRSTIGKAVEFGVPVPAFSTALSFYDGYRSAKLPANLLQAQRDYFGAHTFQVLPGEENDFLKKDQWIHVNWTGKGGNVSASTYDA

SEQ ID NO: 119 - DNA, 1470 bp, Aspergillus nidulans
atgacggaagatgctgatttcggtctcatcggtctggccgttatgggccagaacctgatcctcaacgctgctgatcacggtttcactgtctgcgcctacaaccgtaccacctccaaggttgaccgtttccttgagaacgaggctaagggcaagtctattgttggcgctcactccgtagaggaattctgctccaagctcaagcgccctcgccgtatcatgctccttgtcatggccggaaaccccgttgaccagttcatcgagtctcttctgccccacctcgaggagggtgatattatcatcgacggtggtaactcccacttccccgacagcaaccgccgcaccaagtacctgaaggagaagggcatccgcttcgtcggcagcggtgtctccggtggtgaggagggtgcccgctacggtccctctctcatgcccggtggtaacgaggaggcctggccttacatcaaggatatcttccagagcatcgccgccaagagcgatggcgaggcttgctgcgactgggtcggtgatgagggtgctggtcactacgtcaagatggtccacaatggtattgagtacggtgacatgcagctcatctgcgaggcttacgacatcctcaagcgtggtgtcggcctctcctccaaggagattgccgatgttttcgccaagtggaacaacggtgttcttgactctttcctgattgaaatcactcgtgacattctctacttcaacgacgacgatggaacccccatggttgagaagatcctcgacaaggctggtcagaagggtaccggcaagtggaccgccgtcaacgctctcgacctcggcatgcccgtcaccctgattggcgagtccgtcttcgctcgctgcctctctgctctcaaggacgagcgtatccgcgccagcagcctcctcaacggccccactcccgagttcaccggtgacaaggaggagttcattgccgatctcgagcaggctctctacgcctccaagatcatctcctacgcccagggtttcatgctcatccagaacgctgccaaggagtacggctggaagctcaacaagcccgccattgccctcatgtggcgcggtggttgcatcatccgctccgtcttcctgaaggacatcaccgaggcttaccgcaaggaccccgacctggagaacctgctcttcaacgacttcttcaacaaggccatccacaacgcccagaagggctggagaaacgttgtcagcaagggtgctctctggggtatccccactcccgccttcagcaccgctctgtccttctacgacggctaccgcacaaaggctctccccgccaacctgttgcaggctcagcgtgactacttcggtgcccacaccttccgcatcaagcccgagcacgccagcgagaagtaccccgctgacaaggacatccacgtcaactggaccggccgtggtggtcaagtttccgcctcgacctacactgcttaa

SEQ ID NO: 120 - protein, 489 aa, Aspergillus nidulans
MTEDADFGLIGLAVMGQNLILNAADHGFTVCAYNRTTSKVDRFLENEAKGKSIVGAHSVEEFCSKLKRPRRIMLLVMAGNPVDQFIESLLPHLEEGDIIIDGGNSHFPDSNRRTKYLKEKGIRFVGSGVSGGEEGARYGPSLMPGGNEEAWPYIKDIFQSIAAKSDGEACCDWVGDEGAGHYVKMVHNGIEYGDMQLICEAYDILKRGVGLSSKEIADVFAKWNNGVLDSFLIEITRDILYFNDDDGTPMVEKILDKAGQKGTGKWTAVNALDLGMPVTLIGESVFARCLSALKDERIRASSLLNGPTPEFTGDKEEFIADLEQALYASKIISYAQGFMLIQNAAKEYGWKLNKPAIALMWRGGCIIRSVFLKDITEAYRKDPDLENLLFNDFFNKAIHNAQKGWRNVVSKGALWGIPTPAFSTALSFYDGYRTKALPANLLQAQRDYFGAHTFRIKPEHASEKYPADKDIHVNWTGRGGQVSASTYTA

SEQ ID NO: 121 - DNA, 1476 bp, Aspergillus oryzae
atggctgaccaggcagttgctgacttcggtttgatcggtttggccgtcatgggccagaacctgatcatgaacgtcgccgatcacggtttcactgtttgcgcttacaaccgcacaacctccaaggttgaccgcttccttgaaaatgaggccaagggcaagtccattgtcggtgctcactccattgaggagttctgcgctaagctgaagcgtccccgccgtatcatgctcctggttatggctggaaagcccgtcgaccagttcattgagtctctcctgccccaccttgagaagggcgatatcatcattgatggtggtaactcccacttccccgacagcaaccgccgtaccaagtaccttgccgagaagggcatccgcttcgtcggtagcggtgtctctggtggtgaggagggtgcccgttacggtccctctctcatgcccggtggtaacgaggaggcctggcctttcattaaggatatcttccagagcattgccgccaagagcgaaggtgaggcttgctgcgactgggtcggtgatgagggtgccggccactacgtcaagatggtccacaacggtatcgagtatggtgacatgcagctgatctgtgaggcctatgatattctcaagcgtggtgttggcatgcccgtcaacgaaatcgccgaagtcttcgacaagtggaacaagggtgtcttggactccttcttgatcgaaatcacccgcgatgtcctccgcttcaacgatgacgacggcactcccctcgttgagaagatccttgacaaggccggccagaagggaaccggcaagtggaccgccatcaacgctcttgaccttggtatgcctgtcaccctgatcggtgaggctgtcttctctcgttgcctcagtgcccttaaggacgagcgtgtccgcgctagcagcctcctcccaggccccactcctcaattcaccggtgacaagcaggctttcgtcgatgatctggagcaggccctttatgcttctaagatcatctcctatgcccagggcttcatgctcatgcaggaggctgccaaggagtacggctggaagcttaacaagccttccatcgcccttatgtggcgtggtggctgcatcatccgctctgtcttcctgaaggacatcaccaacgcctaccgccagaaccccgaccttgagaacctcctcttcgacaagttcttcaacgaagccatcgccaaggcccagaacggctggagaaacgttgtcagcaagggtgctctctggggtatccctactcccgctttcagcactgctctcagcttctacgacggataccgcactcgggacctccccgccaacctgctgcaggctcagcgtgactacttcggtgcccacaccttccgcgtcaagcccgagcacgccaacgagacctaccctgagggcaaggacatccacgtcaactggactggacgtggtggtaatgtgtccgcctctacctacattgcttaa

SEQ ID NO: 122 - protein, 491 aa, Aspergillus oryzae
MADQAVADFGLIGLAVMGQNLIMNVADHGFTVCAYNRTTSKVDRFLENEAKGKSIVGAHSIEEFCAKLKRPRRIMLLVMAGKPVDQFIESLLPHLEKGDIIIDGGNSHFPDSNRRTKYLAEKGIRFVGSGVSGGEEGARYGPSLMPGGNEEAWPFIKDIFQSIAAKSEGEACCDWVGDEGAGHYVKMVHNGIEYGDMQLICEAYDILKRGVGMPVNEIAEVFDKWNKGVLDSFLIEITRDVLRFNDDDGTPLVEKILDKAGQKGTGKWTAINALDLGMPVTLIGEAVFSRCLSALKDERVRASSLLPGPTPQFTGDKQAFVDDLEQALYASKIISYAQGFMLMQEAAKEYGWKLNKPSIALMWRGGCIIRSVFLKDITNAYRQNPDLENLLFDKFFNEAIAKAQNGWRNVVSKGALWGIPTPAFSTALSFYDGYRTRDLPANLLQAQRDYFGAHTFRVKPEHANETYPEGKDIHVNWTGRGGNVSASTYIA

SEQ ID NO: 123 - DNA, 1470 bp, Yarrowia lipolytica
atgactgacacttcaaacatcaagcctgtcgctgacattgccctcatcggtctcgccgtcatgggccagaacctgatcctcaacatggccgaccacggctacgaggttgttgcctacaaccgaaccacctccaaggtcgaccacttcctcgagaacgaggccaagggaaagtccattattggtgctcactctatcaaggagctgtgtgctctgctgaagcgaccccgacgaatcattctgctcgttaaggccggtgctgctgtcgattctttcatcgaacagctcctgccctatctcgataagggtgatatcatcattgacggtggtaactcccacttccccgactccaaccgacgatacgaggagcttaacgagaagggaatcctctttgttggttccggtgtttccggcggtgaggagggtgcccgatacggtccctccatcatgcccggtggaaacaaggaggcctggccccacattaagaagattttccaggacatctctgctaaggctgatggtgagccctgctgtgactgggtcggtgacgctggtgccggccactttgtcaagatggttcacaacggtattgagtatggtgacatgcagcttatctgcgaggcttacgacctcatgaagcgaggtgctggtttcaccaatgaggagattggagacgttttcgccaagtggaacaacggtatcctcgactccttcctcattgagatcacccgagacatcttcaagtacgacgacggctctggaactcctctcgttgagaagatctccgacactgctggccagaagggtactggaaagtggaccgctatcaacgctcttgaccttggtatgcccgtcaccctgatcggtgaggccgtcttcgctcgatgcctttctgccctcaagcaggagcgtgtccgagcttccaaggttcttgatggccccgagcccgtcaagttcactggtgacaagaaggagtttgtcgaccagctcgagcaggccctttacgcctccaagatcatctcttacgcccagggtttcatgcttatccgagaggccgccaagacctacggctgggagctcaacaacgccggtattgccctcatgtggcgaggtggttgcatcatccgatccgtcttccttgctgacatcaccaaggcttaccgacaggaccccaacctcgagaacctgctgttcaacgacttcttcaagaacgccatctccaaggccaacccctcttggcgagctaccgtggccaaggctgtcacctggggtgttcccactcccgcctttgcctcggctctggctttctacgacggttaccgatctgccaagctccccgctaacctgctccaggcccagcgagactacttcggcgcccacacctaccagctcctcgatggtgatggaaagtggatccacaccaactggaccggccgaggtggtgaggtttcttcttccacttacgatgcttaa

SEQ ID NO: 124 - protein, 489 aa, Yarrowia lipolytica
MTDTSNIKPVADIALIGLAVMGQNLILNMADHGYEVVAYNRTTSKVDHFLENEAKGKSIIGAHSIKELCALLKRPRRIILLVKAGAAVDSFIEQLLPYLDKGDIIIDGGNSHFPDSNRRYEELNEKGILFVGSGVSGGEEGARYGPSIMPGGNKEAWPHIKKIFQDISAKADGEPCCDWVGDAGAGHFVKMVHNGIEYGDMQLICEAYDLMKRGAGFTNEEIGDVFAKWNNGILDSFLIEITRDIFKYDDGSGTPLVEKISDTAGQKGTGKWTAINALDLGMPVTLIGEAVFARCLSALKQERVRASKVLDGPEPVKFTGDKKEFVDQLEQALYASKIISYAQGFMLIREAAKTYGWELNNAGIALMWRGGCIIRSVFLADITKAYRQDPNLENLLFNDFFKNAISKANPSWRATVAKAVTWGVPTPAFASALAFYDGYRSAKLPANLLQAQRDYFGAHTYQLLDGDGKWIHTNWTGRGGEVSSSTYDA

SEQ ID NO: 125 - DNA, 1479 bp, Ustilago maydis
atgtcgtcccaagctgttgccgatatcggcctcatcggccttgccgtcatgggtcagaacctgatcctcaacatgaacgacaagggcttcaccgtctgcgcctacaaccgtaccaccagcaaggtggacgacttcctcgccaacgaggctaagggcaccaacgtcgttggcgccaagtcgatcgaggaattcgttgccaagcttaagcgccctcgcaagatgattctcctcgtcaaggccggtcccgccgtcgacgctttcattgagcagctcctcccccacctcgagcagggtgacattgtcattgacggtggtaactcgcactaccccgactcgatccgccgctgcaaggagctcgaggccaagggaatcctctttgtcggctccggtgtatccggtggtgaggagggtgcgcgtcacggcccttcgctcatgcccggtggttcggatgctgcatggccccacatcaaggagattttccagaagaccgctgctcagtcggatggcgagccatgctgtgactgggtcggccaaaccggtgccggccactacgtcaagatggtccacaacggtatcgagtacggcgatatgcagctcatctgcgaggcctacgacatccttaagcacggccttggtctcaaggagggcgagattggtgacattttcaccaagtggaacactggtgtcctcgactcgttcctcatcgagatcacccgtgacattctcaagtacaacgacgaggatggcgcccctctgctcaccaagatcatggactcggccggacagaagggtaccggtaagtggaccgccatcaacgcgctcgacctcggccagcccgtcaccctcatcggcgaggccgtctttgcccgatgcctttcgtcgctcaagggcgagcgtactcgtgcctccaagatcctcggcggtccccagatcaagcccttcgagggtaacaaggagcagttcatcgccgacctcgagcaggctctatacgcttccaagattgtctcgtacgcccaaggcttcatgttgatgcgcgaggccgccaaggagtacgactggaagctcaacaacccctcgatcgcgctcatgtggcgtggtggttgcatcatccgatccgtcttcctcaaggacatcactgcggccttccgcaagaaccccgagctggagaacttgctcttcgacgacttcttcaacaaggctatccacaatgctcaggagggctggcgccgcgtggtcgcccaggccatcctgtggggtatccccaccccggccttctcgaccgcccttgccttctttgacggctaccgtcgcgagctgctccccgccaacctgctgcaggcccagcgtgactactttggtgctcacaccttccgtgttctgccccagttcgccagcgaaaagcttcccgagggccaggacatccacatcaactggacaggccgtggtggtaacgtctcggcttccacctaccaggcataa

SEQ ID NO: 126 - protein, 492 aa, Ustilago maydis
MSSQAVADIGLIGLAVMGQNLILNMNDKGFTVCAYNRTTSKVDDFLANEAKGTNVVGAKSIEEFVAKLKRPRKMILLVKAGPAVDAFIEQLLPHLEQGDIVIDGGNSHYPDSIRRCKELEAKGILFVGSGVSGGEEGARHGPSLMPGGSDAAWPHIKEIFQKTAAQSDGEPCCDWVGQTGAGHYVKMVHNGIEYGDMQLICEAYDILKHGLGLKEGEIGDIFTKWNTGVLDSFLIEITRDILKYNDEDGAPLLTKIMDSAGQKGTGKWTAINALDLGQPVTLIGEAVFARCLSSLKGERTRASKILGGPQIKPFEGNKEQFIADLEQALYASKIVSYAQGFMLMREAAKEYDWKLNNPSIALMWRGGCIIRSVFLKDITAAFRKNPELENLLFDDFFNKAIHNAQEGWRRVVAQAILWGIPTPAFSTALAFFDGYRRELLPANLLQAQRDYFGAHTFRVLPQFASEKLPEGQDIHINWTGRGGNVSASTYQA

SEQ ID NO: 127 - DNA, 1554 bp, Candida albicans
atgaaaaattttaatgccttatcaagattatcaatcttgtcaaaacaattatcgttcaataatactaacagttctattgctagaggtgatatcggtttaattggtttagccgttatgggtcaaaacttgattcttaacatggccgaccatggttacactgttgttgcttacaacagaaccactgccaaagttgatcgtttcttagaaaacgaagctaaaggtaaatccatcctcggtgctcactccatcaaggaattggttgatcaattaaagagaccaagaagaattatgcttttggtcaaagctggtgctccagttgatgaattcattaaccaattattgccatacttggaagaaggtgatatcatcattgacggtggtaactcccatttcccagattctaacagaagatacgaagaattggccaagaaaggtattttgtttgttggttccggtgtttctggtggtgaagaaggtgctagaactggtccatctttgatgccaggtggtaacgaaaaagcttggccacacattaaagacatcttccaagatgttgccgccaagagtgatggtgaaccatgttgtgactgggttggtgatgccggtgctggtcattacgtcaagatggtccacaatggtattgaatatggtgatatgcaattgatttgtgaagcttacgatcttatgaagagagttggtaaatttgaagacaaagaaattggtgacgtgtttgccacatggaacaaaggtgttttggattctttcttgattgaaatcaccagagacattttatactacaatgacccaactgacggtaaaccattggttgaaaaaatcttggatactgctggtcaaaaaggtactggtaaatggactgctgtcaatgcccttgatttgggtatcccagttactttgattggtgaagctgtcttttctagatgtctttctgccatgaaagccgaaagagttgaagcctctaaagccttgaaaggtccacaagttactggtgaatctccaattactgacaaaaaacaatttattgatgatttagaacaagctttgtatgcttccaagattatctcctacacccaaggtttcatgttgatgaaccaagctgctaaggattacggctggaaattgaacaatgctggtattgccttgatgtggagaggtggttgtattatcagatcagttttcttggctgaaattactgctgcttacagaaagaaaccagacttggaaaacttgttgctttacccattctttaacgacgctatcactaaagctcaatctggatggagagcttctgttggtaaggctattcaatatggtattccaactccagctttctctactgctttggctttctacgatggtttaagatctgaaagattaccagctaacttgttacaagctcaaagagattactttggtgctcatactttcaaggtcttgccaggtcaagaaaatgaattgttgaagaaagatgaatggattcacattaactggactggtagaggtggtgatgtttcttcaactacttacgatgcttag

SEQ ID NO: 128 - protein, 517 aa, Candida albicans
MKNFNALSRLSILSKQLSFNNTNSSIARGDIGLIGLAVMGQNLILNMADHGYTVVAYNRTTAKVDRFLENEAKGKSILGAHSIKELVDQLKRPRRIMLLVKAGAPVDEFINQLLPYLEEGDIIIDGGNSHFPDSNRRYEELAKKGILFVGSGVSGGEEGARTGPSLMPGGNEKAWPHIKDIFQDVAAKSDGEPCCDWVGDAGAGHYVKMVHNGIEYGDMQLICEAYDLMKRVGKFEDKEIGDVFATWNKGVLDSFLIEITRDILYYNDPTDGKPLVEKILDTAGQKGTGKWTAVNALDLGIPVTLIGEAVFSRCLSAMKAERVEASKALKGPQVTGESPITDKKQFIDDLEQALYASKIISYTQGFMLMNQAAKDYGWKLNNAGIALMWRGGCIIRSVFLAEITAAYRKKPDLENLLLYPFFNDAITKAQSGWRASVGKAIQYGIPTPAFSTALAFYDGLRSERLPANLLQAQRDYFGAHTFKVLPGQENELLKKDEWIHINWTGRGGDVSSTTYDA

SEQ ID NO: 129 - DNA, 1554 bp, Candida albicans
atgaaaaattttaatgccttatcaagattatcaatcttgtcaaaacaattatcgttcaataatactaacagttctattgctagaggtgatatcggtttaattggtttagccgttatgggtcaaaacttgattcttaacatggccgaccatggttacactgttgttgcttacaacagaaccactgccaaagttgatcgtttcttagaaaacgaagctaaaggtaaatccatcctcggtgctcactccatcaaggaattggttgatcaattaaagagaccaagaagaattatgcttttggtcaaagctggtgctccagttgatgaattcattaaccaattattgccatacttggaagaaggtgatatcatcattgacggtggtaactcccatttcccagattctaacagaagatacgaagaattggccaagaaaggtattttgtttgttggttccggtgtttctggtggtgaagaaggtgctagaactggtccatctttgatgccaggtggtaacgaaaaagcttggccacacattaaagagatcttccaagatgttgccgccaagagtgatggtgaaccatgttgtgactgggttggtgatgccggtgctggtcattacgtcaagatggtccacaatggtattgaatatggtgatatgcaattgatttgtgaagcttacgatcttatgaagagagttggtaaatttgaagacaaagaaattggtgacgtgtttgccacatggaacaaaggtgttttggattctttcttgattgaaatcaccagagacattttatactacaatgacccaactgacggtaaaccattggttgaaaaaatcttggatactgctggtcaaaaaggtactggtaaatggactgctgtcaatgcccttgatttgggtatcccagttactttgattggtgaagctgtcttttctagatgtctttctgccatgaaagccgaaagagttgaagcctctaaagccttgaaaggtccacaagttactggtgaatctccaattactgacaaaaaacaatttattgatgatttagaacaagctttgtatgcttccaagattatctcctacacccaaggtttcatgttgatgaaccaagctgctaaggattacggctggaaattgaacaatgctggtattgccttgatgtggagaggtggttgtattatcagatcagttttcttggctgaaattactgctgcttacagaaagaaaccagacttggaaaacttgttgctttacccattctttaacgacgctatcactaaagctcaatctggatggagagcttctgttggtaaggctattcaatatggtattccaactccagctttctctactgctttggctttctacgatggtttaagatctgaaagattaccagctaacttgttacaagctcaaagagattactttggtgctcatactttcaaggtcttgccaggtcaagaaaatgaattgttgaagaaagatgaatggattcacattaactggactggtagaggtggtgatgtttcttcaactacttacgatgcttag

SEQ ID NO: 130 - protein, 517 aa, Candida albicans
MKNFNALSRLSILSKQLSFNNTNSSIARGDIGLIGLAVMGQNLILNMADHGYTVVAYNRTTAKVDRFLENEAKGKSILGAHSIKELVDQLKRPRRIMLLVKAGAPVDEFINQLLPYLEEGDIIIDGGNSHFPDSNRRYEELAKKGILFVGSGVSGGEEGARTGPSLMPGGNEKAWPHIKEIFQDVAAKSDGEPCCDWVGDAGAGHYVKMVHNGIEYGDMQLICEAYDLMKRVGKFEDKEIGDVFATWNKGVLDSFLIEITRDILYYNDPTDGKPLVEKILDTAGQKGTGKWTAVNALDLGIPVTLIGEAVFSRCLSAMKAERVEASKALKGPQVTGESPITDKKQFIDDLEQALYASKIISYTQGFMLMNQAAKDYGWKLNNAGIALMWRGGCIIRSVFLAEITAAYRKKPDLENLLLYPFFNDAITKAQSGWRASVGKAIQYGIPTPAFSTALAFYDGLRSERLPANLLQAQRDYFGAHTFKVLPGQENELLKKDEWIHINWTGRGGDVSSTTYDA

SEQ ID NO: 131 - DNA, 1479 bp, Schizosaccharomyces pombe
atgtcacaaaaagaagttgccgattttggtctcatcggtttggccgtcatgggtcaaaacttgattctcaacggtgccgacaagggctttaccgtctgttgctacaaccgtacaacttccagagttgacgagtttttggcaaacgaagccaaaggtaaatctattgttggtgctcactcccttgaggaatttgtttccaagcttaagaagcctcgtgtttgtattttgcttgttaaggctggtaagcctgttgattaccttattgagggattggctcctctcctcgagaagggcgatatcattgttgacggtggtaactcccattaccctgatactacccgccgttgcgaggagttggccaagaagggcattctttttgttggttccggtgtttctggtggtgaagagggtgctcgttacggtccctctttgatgcctggtggtaatcccgctgcctggcctcgtatcaagcccatcttccaaactttggccgccaaagccggaaacaacgagccttgctgtgactgggttggtgaacaaggtgctggtcattatgttaagatggttcacaatggtatcgaatatggtgacatgcaattgatttgcgaaacttatgacattatgaaacgtggccttggtatgtcttgtgatgagattgccgatgtttttgagaagtggaacactggtaaacttgactctttcttaattgaaatcactcgcgatgttttgcgttacaaggctgatgacggcaagcccttggttgagaagattctcgacgctgctggacagaagggtactggtaagtggactgctcaaaacgctttggaaatgggtacccctgtcagtttgatcaccgaagccgtttttgctcgttgtctttcctccttgaagagcgagcgtgttcgtgcctctaagaagcttactggccccaacaccaagttcactggtgacaagaagcaattgattgatgatttggaagatgccttgtacgcttccaagattatctcttatgctcaaggtttcatgcttatgcgtgaggctgctaaggagtacggctggaagcttaacaacgccggtatcgctttgatgtggcgtggtggttgtattatccgttccgtcttccttaaggatatcactgaggctttccgtgaggatcctaacctcgagtctatccttttccatcctttcttcactaatggtgttgaaaaggctcaagctggttggcgtagagttgttgcccaagccgctatgttgggcattccagtccctgccacttctaccggtctttccttctacgacggttatcgtagtgctgttttgcctgctaacttattgcaagctcaacgtgactactttggtgcccatactttccgtgttttgcctgaagctgctgataagagcttacctgctgacaaggatatccacattaactggaccggtcacggtggtaacatcagtgctactacctatgatgcttag

SEQ ID NO: 132 - protein, 492 aa, Schizosaccharomyces pombe
MSQKEVADFGLIGLAVMGQNLILNGADKGFTVCCYNRTTSRVDEFLANEAKGKSIVGAHSLEEFVSKLKKPRVCILLVKAGKPVDYLIEGLAPLLEKGDIIVDGGNSHYPDTTRRCEELAKKGILFVGSGVSGGEEGARYGPSLMPGGNPAAWPRIKPIFQTLAAKAGNNEPCCDWVGEQGAGHYVKMVHNGIEYGDMQLICETYDIMKRGLGMSCDEIADVFEKWNTGKLDSFLIEITRDVLRYKADDGKPLVEKILDAAGQKGTGKWTAQNALEMGTPVSLITEAVFARCLSSLKSERVRASKKLTGPNTKFTGDKKQLIDDLEDALYASKIISYAQGFMLMREAAKEYGWKLNNAGIALMWRGGCIIRSVFLKDITEAFREDPNLESILFHPFFTNGVEKAQAGWRRVVAQAAMLGIPVPATSTGLSFYDGYRSAVLPANLLQAQRDYFGAHTFRVLPEAADKSLPADKDIHINWTGHGGNISATTYDA

SEQ ID NO: 133 - DNA, 1536 bp, Aspergillus fumigatus
atgacttctatccccacggtcttcaatccgctacacgacttatgcggcgttgctatatcttgcgctggacaaagctgtgctgatttcggtctcatcggtctggccgttatgggccagaacctgatccttaacgttgctgatcacggtttcacggtctgcgcttacaaccgtacgacctccaaggtcgaccgcttcctagcaaatgaggccaagggcaagtccattgtcggtgctcactccgtcgaggaattctgcgccaagctgaagcgccctcgccgtatcatgctgctggttatggctggcaagcctgtcgacgacttcattgagtctctcctcccccaccttgaggagggtgacatcatcatcgatggtggtaactcccacttccccgacagcaaccgccgcaccaagtacctgaaggagaagggcatccgcttcgtcggcagcggtgtttccggtggtgaggagggtgcccgctacggtccctctctgatgcccggtggcaacgaggaggcctggcccttcatcaaggacatcttccagagcattgccgccaagagcgacggcgaggcttgctgtgactgggtgggtgacgagggcgctggtcacttcgtcaagatggtccacaacggtatcgagtacggtgacatgcagctgatctgcgaggcttatgacatcatgaagcgtggtctgggcatgcccgtcaacgagatcgccgatgtcttcgctaagtggaacaagggtgtgctggactcgttcctgattgagatcactcgtgacgttctctacttcaacgacaacgatggcactcccctcgtcgagaagatccttgacaaggcgggccagaagggtaccggcaagtggaccgccatcaacgctctcgaccttggcatgcccgttaccctgatcggtgaggccgtcttcgcccgttgcctgagcgccatcaaggacgagcgtatccgtgccagcagcctcctcgatggccctactccccagttcaccggggacaagcaggctttcatcgacgacctcgagcaggctctgtatgcctccaagatcatctcctacgctcagggcttcatgctcatccaggaggctgctcgcgagtacggctggaagctgaacaagccgtccattgccctcatgtggcgtggtggctgcattatccgttccgtcttcctcaaggacatcaccaacgcttaccgcaacaacccggacctggagaacctcctgttcgacgacttcttcaaggccgccatccagaaggctcagcagggctggagaaacgtggtcagcaagggcgccctctggggtatccccactcccgccttcagcactgctctgagcttctacgacggataccgcacccgagacctccctgccaacctgctgcaggctcagcgtgactacttcggtgctcacaccttccgcgtcaagcccgagtgcgccaacgagaactaccccgagggcaaggatatccacgtcaactggaccggtcgtggtggtgacgtgtccgcctccacctacgttgtttaa

SEQ ID NO: 134 - protein, 511 aa, Aspergillus fumigatus
MTSIPTVFNPLHDLCGVAISCAGQSCADFGLIGLAVMGQNLILNVADHGFTVCAYNRTTSKVDRFLANEAKGKSIVGAHSVEEFCAKLKRPRRIMLLVMAGKPVDDFIESLLPHLEEGDIIIDGGNSHFPDSNRRTKYLKEKGIRFVGSGVSGGEEGARYGPSLMPGGNEEAWPFIKDIFQSIAAKSDGEACCDWVGDEGAGHFVKMVHNGIEYGDMQLICEAYDIMKRGLGMPVNEIADVFAKWNKGVLDSFLIEITRDVLYFNDNDGTPLVEKILDKAGQKGTGKWTAINALDLGMPVTLIGEAVFARCLSAIKDERIRASSLLDGPTPQFTGDKQAFIDDLEQALYASKIISYAQGFMLIQEAAREYGWKLNKPSIALMWRGGCIIRSVFLKDITNAYRNNPDLENLLFDDFFKAAIQKAQQGWRNVVSKGALWGIPTPAFSTALSFYDGYRTRDLPANLLQAQRDYFGAHTFRVKPECANENYPEGKDIHVNWTGRGGDVSASTYVV

SEQ ID NO: 135 - DNA, 2367 bp, Saccharomyces cerevisiae
atgaagatggccgtggatgaccctacgtatggcaccccaaagattaaaagaactgcttctagcagtagtagtattgaaacgacagtaacaaagttgctgatgtccaccaagcacctcctccaggtcttaacacaatggtctaaggggacaacctctggaagattagtatctgatgcatacgttcagttgggcaatgacttcaaagtcgtatcgaaattcttcatgcatgcgaaagttgacatgtcagatgttggtgatgtgccaatggctttaagacgtgtattggaagtgacattgagggaaccaccatctgatgaaactttaaataaacatctgccaaaaatcagagagattatagtgacacttttagacaaactaaaggttaaacaagcaattttgaagaatatgcagcaagagcatcggataagtgtaaaatcacatcatcaacaaaacccttcctttactagtaacctttcattgggtagtgaagggacccgtgaggggactccattaagtagcagaaaaagcagtattgtacgtgatcagaggcaaagcgattcagtcgaaaacagctatggggaaaaagtgaattctacttctacgggaacaccctctgctcagtcagcagaggcaacattgacaaaaccaagaactaatattaaacaaaaccttaaatctaacaatgctccaaacgcatctgatgatgatgatgcactttcacaattgaagaagggaactaatttacaaaggagagcctcaaagagatactctgcctaccatatggctaaattgacaaatcagtctacaacagaagctgctgcggctgccggcctcatgacaactccttcaccctcgatgttgcatctagaggaaaccgtaagaaagtcaaagttatacggaaataacaacaatgatgatgatagaaacattaattcagctgaaaataagggtaaaagcattgacgatgtatcaaaggcgagtccattggcgaaaacgccattgcctattgagaatgttcgtgcatctccacgaagattatcaagcgttgttacaacttcgccagataaagccatgaatggaacttgtccagtatttttaaggataggagacaaaacaaagaagtgtcatgtgcaactgcctactacaaaaaacgcattaaggctcctatttattgagcgctttgcgtattctcctggagcaaattccttcccagacatatatattatggatcctcaatatggagtattctatgaattagaggaactaaatcttctcgatattaaagaagggtttgttatcgagctaaagctcgaagagaatccaaataatacgatcaaggaattcattgacacagttaagatggagatatcgaatagtcagaatgatataataagacatttaaaagaaatgagttttggttctgcaattagcggcaagcaaacggaagttttaccacaacctggccttgaagctaacaaacacgatcttgtggggcaaaacaaaaaagatgatgataaaacgatcaaagatattcagtacgaattggggaaaataaaacaagtgcataatattaataggtcaaatattaatgagactattttcaacattttacgaaaggtcgataactttaagtctttatcattttctgccaaaaactcttccaacagaatgtacatggaaaaatcacagaccgaactcggtgatttatccgatactctgctaagtaaagtagatgatttgcaagatgtaatcgaaattatgaggaaagacgttgctgagcgtaggtctcagcccgctaaaaagaagctagaaaccgtatccaaggatttagaaaatgcccaagcagatgtacttaaactacaggagtttattgatactgaaaaaccacattggaaaaaaacttgggaagctgagctcgataaggtctgtgaagaacaacagtttctcacgttgcaggaagagttgattttagatttgaaggaagacttgggtaaggcattagaaactttcgatcttattaagttatgttgtgaagaacaagaaaaaaacccttcgagatcaaaaagtaatcctattttgcctattatgagaccgggcacatttaatcaggtaagggagcaagttatggtggccgtccaatctttaaaccctgaccacgatagtagggtcgaagctattgacaaggctgagaagatgtgggagatggaaagaaaacttaaagctagtaatgaatttgatgatgaactggaaaattttgtgggtaattcaaacctgaaaaaatcgggaggctttgaagaagtagaaaggataagaaagcagaaggatgaagcaaatttgagagcatattttgggccggggtttacttaa

SEQ ID NO: 136 - protein, 788 aa, Saccharomyces cerevisiae
MKMAVDDPTYGTPKIKRTASSSSSIETTVTKLLMSTKHLLQVLTQWSKGTTSGRLVSDAYVQLGNDFKVVSKFFMHAKVDMSDVGDVPMALRRVLEVTLREPPSDETLNKHLPKIREIIVTLLDKLKVKQAILKNMQQEHRISVKSHHQQNPSFTSNLSLGSEGTREGTPLSSRKSSIVRDQRQSDSVENSYGEKVNSTSTGTPSAQSAEATLTKPRTNIKQNLKSNNAPNASDDDDALSQLKKGTNLQRRASKRYSAYHMAKLTNQSTTEAAAAAGLMTTPSPSMLHLEETVRKSKLYGNNNNDDDRNINSAENKGKSIDDVSKASPLAKTPLPIENVRASPRRLSSVVTTSPDKAMNGTCPVFLRIGDKTKKCHVQLPTTKNALRLLFIERFAYSPGANSFPDIYIMDPQYGVFYELEELNLLDIKEGFVIELKLEENPNNTIKEFIDTVKMEISNSQNDIIRHLKEMSFGSAISGKQTEVLPQPGLEANKHDLVGQNKKDDDKTIKDIQYELGKIKQVHNINRSNINETIFNILRKVDNFKSLSFSAKNSSNRMYMEKSQTELGDLSDTLLSKVDDLQDVIEIMRKDVAERRSQPAKKKLETVSKDLENAQADVLKLQEFIDTEKPHWKKTWEAELDKVCEEQQFLTLQEELILDLKEDLGKALETFDLIKLCCEEQEKNPSRSKSNPILPIMRPGTFNQVREQVMVAVQSLNPDHDSRVEAIDKAEKMWEMERKLKASNEFDDELENFVGNSNLKKSGGFEEVERIRKQKDEANLRAYFGPGFT

SEQ ID NO: 137 - DNA, 990 bp, Saccharomyces cerevisiae
atgaaattttctaaagtctcactactggccgcatctgcatctgtcgctttatctgccccagttgctgtgactgtcacgcaacatgtccaccaagctgccactgtagtggtacaaggtatagttcgtgtagaaaatggccagactctaacgacgttcattaccaagggcactcaaacggcttctgctagtcccgttgccacaacatcagctcctattgtggttgctaatgctcaagtggacagcatagctacttccgttatccaagaaagcgctgtcgtggctgaatctgcaacatttgaagaatcctctacagaaacatctgaagcattttctacagcaactgcaaccatacaagcggtgcaaacctccgcaagtgccacacaggatgatgtaaccacaactttaacatcttcaacacaacctaccagcacaactactccaacaaccactaccactagcccaaccactaccactagcccaactactaccgctagtccaactactactgctagcccaaccaccgctaccaccactcaatctaccgcctcaagcactcaatcctcaagctccgatttctcaacgtcaatggttaacgaacacaacactaaaagggcgttgcacaaggataccggttctttgacatggtctgacacactagcaacatatgcacaaaactacgctgactcctacgattgttcaggcaacctagtccactccggcggtccatacggtgaaaacttggcacttggttacggtacgaccggctctgttgatgcctggtataacgaaattaccagctacgactattccaaccctggttttagtgaaagcgcaggtcacttcacccaagtcgtatggaagggaacttctgaagttggttgcggtttgaaatcttgcggtggcgaatggggcgattacatcatttgttcctacaaagccgctggtaatgtcattggtgagtttgctgacaacgttatgccattagcttaa

SEQ ID NO: 138 - protein, 329 aa, Saccharomyces cerevisiae
MKFSKVSLLAASASVALSAPVAVTVTQHVHQAATVVVQGIVRVENGQTLTTFITKGTQTASASPVATTSAPIVVANAQVDSIATSVIQESAVVAESATFEESSTETSEAFSTATATIQAVQTSASATQDDVTTTLTSSTQPTSTTTPTTTTTSPTTTTSPTTTASPTTTASPTTATTTQSTASSTQSSSSDFSTSMVNEHNTKRALHKDTGSLTWSDTLATYAQNYADSYDCSGNLVHSGGPYGENLALGYGTTGSVDAWYNEITSYDYSNPGFSESAGHFTQVVWKGTSEVGCGLKSCGGEWGDYIICSYKAAGNVIGEFADNVMPLA

SEQ ID NO: 139 - DNA, 1896 bp, Escherichia coli
atgtctgcaacaaaactgacccgccgcgaacaacgcgcccgggcccaacattttatcgacaccctggaaggcaccgcctttcccaactcaaaacgcatttatatcactggcacacaccccggcgtgcgcgtgccgatgcgtgagatccagcttagcccgacgctaattggcggtagcaaagaacagccgcagtacgaagaaaacgaagcgattccggtctacgacacctccggcccgtatggtgatccgcagattgccattaacgtgcagcaagggctggcaaaactacgccagccgtggatcgatgcgcgcggcgataccgaagaacttaccgtgcgcagttccgattacactaaagcgcggctggcagatgatggcctcgacgaactgcgttttagcggcgtactaacaccaaaacgcgccaaagcaggacgccgtgtcacccaactgcactacgcccgccagggcatcatcacgccggaaatggaattcatcgccatccgcgagaatatgggccgcgagcgcatccgtagcgaggttttacgccaccagcatccgggaatgagctttggcgcacatctgccggaaaatatcactgcggaatttgtccgtgatgaagttgctgccggacgtgcgattatcccggccaacattaatcatccggaatcggagccgatgattattggtcgcaatttcctggtaaaagttaacgccaatatcggcaactcggcggtcacctcttccatcgaagaagaagtggaaaagctggtatggtccacgcgctggggagcggatacggtgatggatctctccaccggtcgctatattcacgaaacccgcgagtggattttgcgtaacagcccggtgccgatcggtacagtgccgatctaccaggcgctggagaaggttaacgggatcgccgaagatcttacctgggaagcgttccgcgacacgctgctggaacaggccgagcaaggtgtggattacttcactatccatgcgggcgtactgctgcgctatgtgccgatgaccgcgaaacgcctgaccggtatcgtctctcgcggcggttcgattatggcgaaatggtgcctctcccatcatcaggaaaatttcctctatcaacacttccgcgaaatttgtgaaatctgtgccgcttatgacgtttcgctgtcgctgggcgacggtctgcgccccggttctattcaggacgccaacgatgaagcgcagtttgccgagctgcatacgctgggcgaactgaccaaaattgcctgggaatatgacgtgcaggtgatgattgaaggcccaggccacgtgccgatgcagatgatccgccgcaatatgaccgaggagttagagcactgccacgaagcgccgttttacactctggggccgctaactaccgatattgcgccgggctatgaccacttcacgtcggggattggtgcggcgatgattggctggtttggctgcgcgatgctctgttacgtaacgccaaaagagcatctgggtctgcccaataaagaagatgttaagcaggggcttatcacctataagattgctgcccacgccgctgacctggcgaaagggcatccgggcgcgcaaattcgcgataacgccatgtcgaaagcccgcttcgaatttcgctgggaagaccagtttaatctggccctcgacccgtttaccgcccgcgcttatcacgatgaaaccctgccgcaagagtcaggtaaagtcgcccatttttgctccatgtgtgggccgaaattctgctcgatgaaaatcagccaggaagtgcgtgattacgccgccacgcaaactattgaaatgggaatggcggatatgtcggagaacttccgtgccagaggcggagaaatctacctgcgtaaggaggaagcgtaa

SEQ ID NO: 140 - protein, 631 aa, Escherichia coli
MSATKLTRREQRARAQHFIDTLEGTAFPNSKRIYITGTHPGVRVPMREIQLSPTLIGGSKEQPQYEENEAIPVYDTSGPYGDPQIAINVQQGLAKLRQPWIDARGDTEELTVRSSDYTKARLADDGLDELRFSGVLTPKRAKAGRRVTQLHYARQGIITPEMEFIAIRENMGRERIRSEVLRHQHPGMSFGAHLPENITAEFVRDEVAAGRAIIPANINHPESEPMIIGRNFLVKVNANIGNSAVTSSIEEEVEKLVWSTRWGADTVMDLSTGRYIHETREWILRNSPVPIGTVPIYQALEKVNGIAEDLTWEAFRDTLLEQAEQGVDYFTIHAGVLLRYVPMTAKRLTGIVSRGGSIMAKWCLSHHQENFLYQHFREICEICAAYDVSLSLGDGLRPGSIQDANDEAQFAELHTLGELTKIAWEYDVQVMIEGPGHVPMQMIRRNMTEELEHCHEAPFYTLGPLTTDIAPGYDHFTSGIGAAMIGWFGCAMLCYVTPKEHLGLPNKEDVKQGLITYKIAAHAADLAKGHPGAQIRDNAMSKARFEFRWEDQFNLALDPFTARAYHDETLPQESGKVAHFCSMCGPKFCSMKISQEVRDYAATQTIEMGMADMSENFRARGGEIYLRKEEA

SEQ ID NO: 141 - DNA, 1371 bp, Saccharomyces cerevisiae
atgtcatttcgtaaaagaggtgaaatactgaacgatagaggtagcgggctaagaggtccactgttaagaggtcctccacgaacttcctcaacgccattgaggacaggcaacagaagagcccctggaaatgtgcccttgtctgacactacagcgagattaaagaagttgaatattgcggacgaatccaagacaaaaatggggctggatagctcccatgtgggcgttagaccttctcctgcaacctctcagccgacgacatcaactgggagtgctgacctggacagtatactaggccatatggggctgccgctgggaaactctgtactagtagaggagcagagcacgacagaatttcactctattctcggtaaactgtttgccgcacagggtatcgttcataatagaatttcagatagcagtgctgataaaactagaaacggcgacactcatgtcattgtcttgtcattgaaccaaatgtttgcaaaggagctacctggtatctacaagggatcacgtaaacaaatgaagaaaaacctaatctctgaagaagaatcaaaagttactgttcaaaatttgaacgagacccagagatcgacgccttccaggtacaaagacctgaaaattgcatggaaatataaactggcagatgaaaagagattgggttctccggatcgagatgacatccaacaaaattctgagtacaaggactacaatcatcagtttgatattacaacgcgtctgatgcccgctcccatagcatctgaactgacatttatcgccccaactcaacctgtctccactattttgagccaaatagaacagactattaaaaggaatgacaagaaactgataagaattgttattccttcccttctacatccggcaatgtatccgcccaaaatgtttgaatcatctgaaataataggtttaatgcacggtgtgaggagtcttgtcaagaaatactatgagcgggttgtactgtttgcttccatatccatagacattatcactcccccattactggtattgctgagaaacatgttcgattctgttatcaatctagaaccctttaaccaggagatgactgagttcttagaacgtgtttataaatcgcaaccggggaagattcaacatgggctagtccacatattgaaattaccagttttcacggaccgtggagagatgagagttttaaagtctgaatgggcatttaagaatggcagaaaaaagtttgaaatagaacaatggggcatacccgtggatgatgctgaaggttcagctgcctcggaacagtcacattcacactcgcactcggatgaaatctcacataatatacctgcaaagaagaccaagatatctttagactattaa

SEQ ID NO: 142 - protein, 456 aa, Saccharomyces cerevisiae
MSFRKRGEILNDRGSGLRGPLLRGPPRTSSTPLRTGNRRAPGNVPLSDTTARLKKLNIADESKTKMGLDSSHVGVRPSPATSQPTTSTGSADLDSILGHMGLPLGNSVLVEEQSTTEFHSILGKLFAAQGIVHNRISDSSADKTRNGDTHVIVLSLNQMFAKELPGIYKGSRKQMKKNLISEEESKVTVQNLNETQRSTPSRYKDLKIAWKYKLADEKRLGSPDRDDIQQNSEYKDYNHQFDITTRLMPAPIASELTFIAPTQPVSTILSQIEQTIKRNDKKLIRIVIPSLLHPAMYPPKMFESSEIIGLMHGVRSLVKKYYERVVLFASISIDIITPPLLVLLRNMFDSVINLEPFNQEMTEFLERVYKSQPGKIQHGLVHILKLPVFTDRGEMRVLKSEWAFKNGRKKFEIEQWGIPVDDAEGSAASEQSHSHSHSDEISHNIPAKKTKISLDY

SEQ ID NO: 143 - DNA, 1035 bp, Saccharomyces cerevisiae
atgtttaaatcattggctgctgtcttgcctagagctagcaaggcaaagttcctccagaaaaattacgcctccactttagctttcattgaaagctcaaaagatggctctgtttcaaggtcatcattgagtttattggctgctgcacaaaagttgtctaaccctatcacagctgtaatcacaggtagcaaagctgaaaaaactgctgaggcgctaaaatcttcatattcatgcagcaatttagaaaagcttgtcatatttgaagattcaaaattagatacctgtcttcccgaacaactaactccgttattagtgaaactattaaaaggcggcgactattcacattttgttgtctcaaactcctctgttggaaaaagtgttttacctcgggtgggtgcgctcttggacgtccaacctgtttgtgaggttactgtaatcaaagatcctaagacctttataaggccaatttatgcaggtaacattatttctacaatagaatgccaggcagaaaaaaaactgttgattattagggcatcagcttttccaccaattgcagagggtagtatggattctgttaccattgagaagagaactgatattcctccttgtgacttaaatgttacctgggttaaaactattcttaccaagagtgaaaggcctgaacttacttctgcacagaacgtggtaactggtggaagggcactcaaggataaggagacatttgagaagctattatcgccgctagcagatgttttgcacgctgctataggtgccacaagagcttctgttgataatggactatgtgataattctctacaaatcggtcagactggtaaggtagtcgcaccaaatttgtatatagccattggcgtttctggtgcagttcagcatttagcgggaatgaaggattcgaaagttatcgttgccattaacaatgatcctgatgcccctatatttaatgtcgctgactatgggttgcaaggggatctttacaaaatagtccctgaactaactgaaaaacttggtaaatataaataa

SEQ ID NO: 144 - protein, 344 aa, Saccharomyces cerevisiae
MFKSLAAVLPRASKAKFLQKNYASTLAFIESSKDGSVSRSSLSLLAAAQKLSNPITAVITGSKAEKTAEALKSSYSCSNLEKLVIFEDSKLDTCLPEQLTPLLVKLLKGGDYSHFVVSNSSVGKSVLPRVGALLDVQPVCEVTVIKDPKTFIRPIYAGNIISTIECQAEKKLLIIRASAFPPIAEGSMDSVTIEKRTDIPPCDLNVTWVKTILTKSERPELTSAQNVVTGGRALKDKETFEKLLSPLADVLHAAIGATRASVDNGLCDNSLQIGQTGKVVAPNLYIAIGVSGAVQHLAGMKDSKVIVAINNDPDAPIFNVADYGLQGDLYKIVPELTEKLGKYK

SEQ ID NO: 145 - DNA, 1512 bp, Saccharomyces cerevisiae
atgcacctagatctcatacacaagtccttcatcttagtgtggctcatatatatacgagccgcgttggctgaccaattcacatataaagcttgctattcagctagtgatatacgaaaattggggttgacttacaagggtgtttacgagtatcaatcagtttcctactgtcaaaatgaatgccctggtcaggctgtcgttgctcttttcaacggtacaggttgctactgtggtggttccgtagctcagttacagtctttgacgcaggtagattcgagcaaatgtgatgtttcgtgtgctggctggccctaccaaaattgtggtggttcttctgccatgaacgtttatatcaataacgctgcttctaccgcggactcaacttcatctactgctacctcaacatcgacaacatcttcatcaagcacaagtgttagttcaaaaacgtccacgaaattggacacgaaaacttcaactagttcttctgctacgcactcatcctcttcctcgtctacaacctccactaccacttcctcttcagagacaactacttcctcctcttcttcatcctcttcctcttctacgtctactacttctactacttctactacctctagtactacttctacttcttcttctccttccaccacttcctctagcacttctgcctcttccagttcagaaacgtcctctacgcaggcaacttcttcatccaccacatcgacctcatcctcaacttcgacagcaacggtcacttctaccccctcatcgacttcaataggtacttccacgcattacactacccgtgtggtgacgcagtccgtggtttctcaagctaaccaacaagcaagcacgatctttaccaccaggacctctgtttatgcaacagtatcgtctacgtcctcctctactagttctctcttaaacgggaaatcgagctcgtccaagagcaaaggcttgagtggtggcgccatcgcaggtgtcgtagtaggtgtggtttgtggtacagttgccttgttggctctggcgttattctttttcgtatggaaaaaacgtcgccaatcttctcagcatgtggacttggaagagacaaagcagtaccagccgtactcactgggtgatgctgacgctaaccctgttattccaccgtctgcttcgagcactaactggcacataccctctaggaataatacagcattgtccaagaatacagcgtctacttttgccacttatgatctgccgacaagggcgccgggcggcagagattctattattactggtgatgcacataatatcagcaaaagaagtcactttccctcggtagtgtacgaggagcctccttcgatctataacgggaaccaaaggttcagtgctacttcgctgcccgatatgatggaagaaagacaactacacattgtcaaccccgataatgtgagttcaaatattgggagtaatgtctcagatggtgatgatgattacgatgatgcaaaggattccaataatagttctttgcgctga

SEQ ID NO: 146 - protein, 503 aa, Saccharomyces cerevisiae
MHLDLIHKSFILVWLIYIRAALADQFTYKACYSASDIRKLGLTYKGVYEYQSVSYCQNECPGQAVVALFNGTGCYCGGSVAQLQSLTQVDSSKCDVSCAGWPYQNCGGSSAMNVYINNAASTADSTSSTATSTSTTSSSSTSVSSKTSTKLDTKTSTSSSATHSSSSSSTTSTTTSSSETTTSSSSSSSSSSTSTTSTTSTTSSTTSTSSSPSTTSSSTSASSSSETSSTQATSSSTTSTSSSTSTATVTSTPSSTSIGTSTHYTTRVVTQSVVSQANQQASTIFTTRTSVYATVSSTSSSTSSLLNGKSSSSKSKGLSGGAIAGVVVGVVCGTVALLALALFFFVWKKRRQSSQHVDLEETKQYQPYSLGDADANPVIPPSASSTNWHIPSRNNTALSKNTASTFATYDLPTRAPGGRDSIITGDAHNISKRSHFPSVVYEEPPSIYNGNQRFSATSLPDMMEERQLHIVNPDNVSSNIGSNVSDGDDDYDDAKDSNNSSLR

SEQ ID NO: 147 - DNA, 1494 bp, Saccharomyces cerevisiae
atggatgcttcctcggttccaccaaaggtagatgattatggaatgtatacaactgaaatttcacatcataacccaatagagttgaaaaatctgctatcatcatcagactccagaagaaattctcaagatgaagatagtctacctaacaatacaaacttaatcaaagaaattgactggcagggcgagaaagttaaaacatatccactaaactatcaaactgtaccactagtaaagctgcaggtgatagcatgcttgattatgttcgtagtctttggtatgaacgatcaaacagtaggtgcactacttcctacgctaattgaatactatcatatatcgcgggtagatgtctcaaacgtatttatagtccaattatgtggttatgtgatggcgtctttatcaaaagagagattgaataagcactttggtatgagaggtggtatgcttctagcagctggtttgtgtatagtatttcttatcattttagcaactgcaccttctagtttctatgtttgtatgttttgtggccttcctcttggtttagggatcggcatcttagattctaccggtaatgttttaatgggcagtcttttagtccataagaatgaactcatgggtatcatgcatggtctttacggggcagcagctatggttactcctcccttagtttcatattttgttgaatggggtcattggtctctttttttccttattcctctgttcttttctataataggcatgattgtaatcttcccagcttttaaatttgaaactgcaagtaaatacgactacctctgctctgtggaaaacaaagaaagcaacaatgatgtggaagaagcgggtgataactcgctgatggaatctaccaaggcgagcccaggattttttgaacttttaagaaatcctgctattttcttgtactcattgtatttgttcctttacttaggtgctgaaattacaactggttcatggttctttagttatttattggaaactaaatcaagtaataaggttgctatgtcatacatagcggcatcgttttggacaggtttgactgtaggcaggttgtgtctaggatttgttactgaaagattcttcgaaaacgagtataaagcaagcaaagcgtacgcttttttaactctatcttcatacaccttatttgtgcttgttgggttgatcaattcaagctcagttttctatttcgtcgtgttattttttgttgttttttgttgtggtacgtttatcggaccattattcccaaacgcaagtatagttgcattacaagtattgcccaaaagactacatgtgagcggggttggggttgccgtcgccgttggtggttgtggtggtgcagccattccatatttggctggagttattgcacacacagtgggaatccagtatatcccactattgtgttggattatggttgcgttatttacattggaatggacattgtatcctaaattcataaaagggcatgaagaatatttttag

SEQ ID NO: 148 - protein, 497 aa, Saccharomyces cerevisiae
MDASSVPPKVDDYGMYTTEISHHNPIELKNLLSSSDSRRNSQDEDSLPNNTNLIKEIDWQGEKVKTYPLNYQTVPLVKLQVIACLIMFVVFGMNDQTVGALLPTLIEYYHISRVDVSNVFIVQLCGYVMASLSKERLNKHFGMRGGMLLAAGLCIVFLIILATAPSSFYVCMFCGLPLGLGIGILDSTGNVLMGSLLVHKNELMGIMHGLYGAAAMVTPPLVSYFVEWGHWSLFFLIPLFFSIIGMIVIFPAFKFETASKYDYLCSVENKESNNDVEEAGDNSLMESTKASPGFFELLRNPAIFLYSLYLFLYLGAEITTGSWFFSYLLETKSSNKVAMSYIAASFWTGLTVGRLCLGFVTERFFENEYKASKAYAFLTLSSYTLFVLVGLINSSSVFYFVVLFFVVFCCGTFIGPLFPNASIVALQVLPKRLHVSGVGVAVAVGGCGGAAIPYLAGVIAHTVGIQYIPLLCWIMVALFTLEWTLYPKFIKGHEEYF

SEQ ID NO: 149 - DNA, 3117 bp, Saccharomyces cerevisiae
atgaattcagacacgaatgcatttaaggacataagaatttcatgtgttgaactttctaggatcgcttttttacccaccgaatcatttgatccaaattcattaacgctattggcatgtttaaaaaaagttgaagagaagttatcagcatacgaagacgattcactttcgccaaaatttgcagattatgtttttgttcctatcgcgagtttattgaaacagccagctctcggcgagtctcaaacagaatacgtcctactaattatatttcatctcttacggacatgttggtcatctaatggtaaattctccgagcaattgggacaacaactgttccccttaatcacatttttagtcagttctgataaagataatcaaaaattaattactagatcagacgagttcaagtacgctggttgtttagtcttgcatcagtttttcaaatcagtaagatcgcaaaggtaccataaagagttcttttcgaactcaaaacctaatttacttcccgctctaggacattctgttaccattctgttgaaaattctggagcaatcgccccaaaataatgagcttcaattcaaagctttggcatccttagaagtactatttcaggacataatatctgatggtgaaatgctttcctttattcttccggggaacgtttctgttttcgctaagatacttactaaaccaggccgtcaaatacattataaggtgtgtgttcgcacgctagaggttctggcaaaattattggttttggtttacgatgattttagtttggatatcaaagtaaataagttaacagatattcgagagttgagcgatacaaagttaaaacacgagataaatcaatcatttatgtttaatggacctattgttttgttacgcacagatgggaagacgcatagagatacaagttggttgacggcaacttctgggcaaatcaatattgctttggaggcctttatacccaaactattaaaacgtaataatgaatcaattgatgaggcattagcgacatttgtatcaatccttttaactagatgtgaaaattctttaaataattgtgagaaagtgttggtatccactttagtacatttggagcgtgatccgatgtccaaattaccatcgcatttggtaaaattaaaggaagttgtcaatgaagacctgcacaagctaagtgacattattcggtttgaaaatgcagataggttatcatctttatcatttgcaatcactattctagagaaaaataacgaaagagatacaatgatcaatgaggtagttagatgtttatttgaatcgcttaatgaatctattgagccaccaagtttgatcaatcataaagagagaataattgaacaaagtagtcaattaacaactacagtaaatttcgaaaacttggaaagtacgaatgctctgattgctttaccaagattatcagaagacatgtcactcaaactgaaaaaatttacataccatatgggttctttgttgttagaaagacatattctcaacgatgtggtgacagagttgatttcagaacaagttgattcaccaagaacgcagaagatagttgcgctgtggttaagcaccaattttataaaggctatggaaaagcaaccgaaggaggaagaagtctatctacaatttgaatctgatgccaactattcctcttccatggttgaagaagtgtgtttgattgtattggaattttgcaatgaactttctcaagacattagcatggagattgaaggtaaaggcataaaaaaaagtgatgagtttgccgtatgtacagttttattctctattgaaacgatttgtgcggtaatgagagaagaatttcaaccagaattgattgactatatatacacagtagttgacgccttggcatcaccgtccgaagccattaggtatgtgagccaatcctgtgcattgaggatagcagataccctatatcatgggtctatcccaaacatgatcttaagcaacgtagattatcttgtagaatctatttcctcaagattgaattctgggatgactgaacgagtgagccaaatattaatggtgatttgccaattggctggctatgaaaccattgaaaattttaaagatgttattgaaacgatatttaaactactggattattatcatggatatagtgatctctgtcttcaatttttccaactatttaaaatcatcattttggagatgaagaaaaaatatatcaatgatgatgagatgattttgaaaatagctaatcaacacatttcacaaagtactttttcgccctggggtatgaccgattttcagcaagtactcaatatactagataaagagacacaggttaaagatgacataacagacgagaacgacgtcgattttttgaaggatgataatgagcccagtaattttcaagaatatttcgactccaaattaagagagccagatagcgacgacgatgaagaagaaagagaagaagaggtggaaggaagttctaaagagtatactgaccagtggacctcacctataccatcagattcatacaaaatactattgcaaattttgggttatggggaaagattattaacccatccatcaaaacgattaagagtgcaaattcttattgttatgaggcttatatttcccttgctatcaacacagcataatctgttgataagggaggttgctagtacatgggactccatcatacagtgcgtcctatgctctgattattcgatcgttcagcccgcatgttcatgcgtggagcagatgataaaatattctggtgattttgtcgccaagagattcattgaactttggcagaaactgtgtcaggattcttttatattgaaagaactgaggattgatccaacggtgcacaaccatgagaaaaaatcgattagtaagcatgtcaaattcccaccagtaactgaaaatgcgctagtatcgatggtacatatggtgctagaaggggtcaaaattacagagtatttaatttctgaagcggtacttgaacaaataatatattgttgtattcaggttgttccagtggagaaaatatcatctatgtcattgatagtgggggatatcgtgtggaaaataagaaatataaattaa

SEQ ID NO: 150 - protein, 1038 aa, Saccharomyces cerevisiae
MNSDTNAFKDIRISCVELSRIAFLPTESFDPNSLTLLACLKKVEEKLSAYEDDSLSPKFADYVFVPIASLLKQPALGESQTEYVLLIIFHLLRTCWSSNGKFSEQLGQQLFPLITFLVSSDKDNQKLITRSDEFKYAGCLVLHQFFKSVRSQRYHKEFFSNSKPNLLPALGHSVTILLKILEQSPQNNELQFKALASLEVLFQDIISDGEMLSFILPGNVSVFAKILTKPGRQIHYKVCVRTLEVLAKLLVLVYDDFSLDIKVNKLTDIRELSDTKLKHEINQSFMFNGPIVLLRTDGKTHRDTSWLTATSGQINIALEAFIPKLLKRNNESIDEALATFVSILLTRCENSLNNCEKVLVSTLVHLERDPMSKLPSHLVKLKEVVNEDLHKLSDIIRFENADRLSSLSFAITILEKNNERDTMINEVVRCLFESLNESIEPPSLINHKERIIEQSSQLTTTVNFENLESTNALIALPRLSEDMSLKLKKFTYHMGSLLLERHILNDVVTELISEQVDSPRTQKIVALWLSTNFIKAMEKQPKEEEVYLQFESDANYSSSMVEEVCLIVLEFCNELSQDISMEIEGKGIKKSDEFAVCTVLFSIETICAVMREEFQPELIDYIYTVVDALASPSEAIRYVSQSCALRIADTLYHGSIPNMILSNVDYLVESISSRLNSGMTERVSQILMVICQLAGYETIENFKDVIETIFKLLDYYHGYSDLCLQFFQLFKIIILEMKKKYINDDEMILKIANQHISQSTFSPWGMTDFQQVLNILDKETQVKDDITDENDVDFLKDDNEPSNFQEYFDSKLREPDSDDDEEEREEEVEGSSKEYTDQWTSPIPSDSYKILLQILGYGERLLTHPSKRLRVQILIVMRLIFPLLSTQHNLLIREVASTWDSIIQCVLCSDYSIVQPACSCVEQMIKYSGDFVAKRFIELWQKLCQDSFILKELRIDPTVHNHEKKSISKHVKFPPVTENALVSMVHMVLEGVKITEYLISEAVLEQIIYCCIQVVPVEKISSMSLIVGDIVWKIRNIN

SEQ ID NO: 151 - DNA, 1629 bp, Saccharomyces cerevisiae
atgaatagcagtctgcaagttggcatcggaattgatgtaggttcatcaagtgctagaataggtgtatataattactacaatgatgcgctgttagaaatggcgcaggaaccagtcccatactaccaggattcttcaaagaagtcatggaaattctggcaaaaatcaactgaaataattaaagcccttcaaaagtgtcttcagaaactgaacattagagaatatgaggtaaagtcatgtggtgtttctgcaacgtgttcattagctattttcgaaagggatcgcacaagtaacatgctgataccgtatccaaatgaagataatgtcatattttggatggactcttctgccgtaaacgaatgtcaatggctaaatatgcaatgtccacaacagcttttggattatttgggaggaaaatttgtacccgaaatgggcgtccccaaattgaagtactttttagatgaatactctcatttacgagacaaacattttcatatctttgatcttcatcaatacattgcttatgaactgagccgcttgtatgaatggaacattgaaggacttttgggaagagaaaatcttaatggtataggcaatgatggagaggtatctggctggtcatcttcattttacaaaaacattataaatttaccatctaatgtaagcattggaaccactagtcttgttgccaataagcacatttcgacaactgtagttcgcagttgtattgacagttatgcaagttggttcgcagttgcttctcctcatttggaaacatcattgtttatgatagccgggacttctagctgctacatgtatggtactaccatctctgatactagaattcctggagtatggggaccttttgatactatacttgacaacaggggtgatttttctgtttatgctgcgggccagtcgtgtactggtaaattaattgaacatttatttgagagtcatccatgtgccaggaaaattctgaaagatggtgctgatatctatcaagtactcgaacaaaccattcgtgacattgagaagaacaatggtctctcaattcatattcttacgaaagatatgtttttctacggtgattacgagggaaataggactccttttgcagatccacggataaagggttctttcattggcgagtccaccgatacatcaatgttgaatcttacatataaatatatttgtatcttggaatttttatcatttcagacaaaattgataattgatacatttcaaaatgaaaattcaaatatacatatcaaagagttaaggatatctggtagtcaagcgaaaaatgaaagactattatcattgatttccttggttaacaacggtgtagcaataattaaacctaaagagaatgtcgatatgatgggtatcaaaggtgcttacgtgcttgctaagtctgcaaaggaaaagaaacaactagctgacgttataacagagagagatattagtaatgatagcgaaaaattcgaatctcttgctgaataccgacttggaaacgatagcatattactaagaaaattgctttgcgttaaatatcatatacacttagatatggctaagcaacagaagcgttatcacaaactcgtcgacgaagtatttcagcacctctaa

SEQ ID NO: 152 - protein, 542 aa, Saccharomyces cerevisiae
MNSSLQVGIGIDVGSSSARIGVYNYYNDALLEMAQEPVPYYQDSSKKSWKFWQKSTEIIKALQKCLQKLNIREYEVKSCGVSATCSLAIFERDRTSNMLIPYPNEDNVIFWMDSSAVNECQWLNMQCPQQLLDYLGGKFVPEMGVPKLKYFLDEYSHLRDKHFHIFDLHQYIAYELSRLYEWNIEGLLGRENLNGIGNDGEVSGWSSSFYKNIINLPSNVSIGTTSLVANKHISTTVVRSCIDSYASWFAVASPHLETSLFMIAGTSSCYMYGTTISDTRIPGVWGPFDTILDNRGDFSVYAAGQSCTGKLIEHLFESHPCARKILKDGADIYQVLEQTIRDIEKNNGLSIHILTKDMFFYGDYEGNRTPFADPRIKGSFIGESTDTSMLNLTYKYICILEFLSFQTKLIIDTFQNENSNIHIKELRISGSQAKNERLLSLISLVNNGVAIIKPKENVDMMGIKGAYVLAKSAKEKKQLADVITERDISNDSEKFESLAEYRLGNDSILLRKLLCVKYHIHLDMAKQQKRYHKLVDEVFQHL

SEQ ID NO: 153 - DNA, 1035 bp, Saccharomyces cerevisiae
atgacattggcagagttgcttggacggtcgagaatagctcaagtagctaacaatcataaaccattgacatacactggcaaaaaatttcatccaacacatcaaatcatcgaaactaagccatcgacactatacagacaagaatgggggttgaaatccgctattccttcgaaaattaaatctcgatatttggtgtacaacgatttggatacgttggaaagaattaccacattcgaacctagaggcggtacgcaatggaataggctaagatttcaagaaatgggagtaccaattgtgtctaatataggcagacaaaatccatttttcaagtacatatctcgtcctgaggatgagtctcatgctaaattgtccttattcaaagaaatgaaaggagatacagatatttcgcctgctgctatgaagaagcgtttgaagaagataacagcactaattagatcgtttcaagatgaatttaaagagtggctggtggaaaatcatccggatgaattgaaattgaattctaataagctggaagactatgtagtgaagtttctaaacaaaaagttggaaacaaaaacgaataaaaaatttaacactgaaatcattggtacaggtggattatcatacagcttgccaggaaagctaaaaaactcaccaaatggtgtcattcaaaggactgtagtacccggtagaatcttgaacgttgttaaggagaacaacgacaataaatggctggcagctattggtgggtttgtggccgatgtggtatttttccaatcaccacctagttcctttaactccatgggagatttcatcagaatgaaaacgttcttgttcgagatcttggaggcatctatggagaaaaatggttctgtttcgatgcacgctagattactcgaaccacaaaatgacaagaccagagaatttttcaataagagaccaatttataaaccattgacctctagaagggcacgtcgaccatcagtgggaaacattcaagaggccaataaccttttgaatataatcaagggaaattga

SEQ ID NO: 154 - protein, 344 aa, Saccharomyces cerevisiae
MTLAELLGRSRIAQVANNHKPLTYTGKKFHPTHQIIETKPSTLYRQEWGLKSAIPSKIKSRYLVYNDLDTLERITTFEPRGGTQWNRLRFQEMGVPIVSNIGRQNPFFKYISRPEDESHAKLSLFKEMKGDTDISPAAMKKRLKKITALIRSFQDEFKEWLVENHPDELKLNSNKLEDYVVKFLNKKLETKTNKKFNTEIIGTGGLSYSLPGKLKNSPNGVIQRTVVPGRILNVVKENNDNKWLAAIGGFVADVVFFQSPPSSFNSMGDFIRMKTFLFEILEASMEKNGSVSMHARLLEPQNDKTREFFNKRPIYKPLTSRRARRPSVGNIQEANNLLNIIKGN

SEQ ID NO: 155 - DNA, 1008 bp, Saccharomyces cerevisiae
atgttgaggttatctttactgagatcaacagctactttgccagtgaaatgccaacgtcgtgggctaatattacctgcggcggcaatgtacaccttaggctcattaatatttggtaaggaagcaaggttggcggatgccatggaacgtggtgagttacataacaagaacgttgattatgcgaaagaagctgaagagcgtaccgagttacgtattagggccctggctaatactcggccaatggaacctcggtacaacggccatgttccccttcatcggtacgagaaattgctgctgtttgcaatttccggttggaattcatttttccatcctgaagatggttataatattgtacaattgggtgaggcaactgcattgccggtcttcttggagaatttgaagcaaacaatgttaagtgattcctctgggaggcgcattttgaaggaacaacccaatatcacaacagagattttgcatatggacaaactagctaaattgccacataacacgtttgggtatgtatattaccaatggttgaaaagagaaaacgtttctccggacactagagcacctgtcaaatttatcgacgatcctatgcatgcatatatctttaagaggtatagacaatgccacgatttctatcacgctataaccaacatgcctattatcattgagggggagatcaccataaaggctcttgaaggtgccaacctgggcgtcccaatggccattctcggtggtatccttgcacctttacgtttgaaaaaggtgcaaagaaaaagattatataatatatatctcccttgggctgtcagaacaggtttaagctgcaagccattgatcaacgtgtattgggaggaaatgctggagaaggatgttactgctttgaggaaagagctaaagataacactccctccggatctaaggacaatgaggaaggagcgtgcagcccttaggaaggagattgacgcaaaatacaactcacagaaacgagccacgactccagcatga

SEQ ID NO: 156 - protein, 335 aa, Saccharomyces cerevisiae
MLRLSLLRSTATLPVKCQRRGLILPAAAMYTLGSLIFGKEARLADAMERGELHNKNVDYAKEAEERTELRIRALANTRPMEPRYNGHVPLHRYEKLLLFAISGWNSFFHPEDGYNIVQLGEATALPVFLENLKQTMLSDSSGRRILKEQPNITTEILHMDKLAKLPHNTFGYVYYQWLKRENVSPDTRAPVKFIDDPMHAYIFKRYRQCHDFYHAITNMPIIIEGEITIKALEGANLGVPMAILGGILAPLRLKKVQRKRLYNIYLPWAVRTGLSCKPLINVYWEEMLEKDVTALRKELKITLPPDLRTMRKERAALRKEIDAKYNSQKRATTPA

SEQ ID NO: 157 - DNA, 2142 bp, Escherichia coli
atgaacatcattgccattatgggaccgcatggcgtcttttataaagatgagcccatcaaagaactggagtcggcgctggtggcgcaaggctttcagattatctggccacaaaacagcgttgatttgctgaaatttatcgagcataaccctcgaatttgcggcgtgatttttgactgggatgagtacagtctcgatttatgtagcgatatcaatcagcttaatgaatatctcccgctttatgccttcatcaacacccactcgacgatggatgtcagcgtgcaggatatgcggatggcgctctggttttttgaatatgcgctggggcaggcggaagatatcgccattcgtatgcgtcagtacaccgacgaatatcttgataacattacaccgccgttcacgaaagccttgtttacctacgtcaaagagcggaagtacaccttttgtacgccggggcatatgggcggcaccgcatatcaaaaaagcccggttggctgtctgttttatgattttttcggcgggaatactcttaaggctgatgtctctatttcggtcaccgagcttggttcgttgctcgaccacaccgggccacacctggaagcggaagagtacatcgcgcggacttttggcgcggaacagagttatatcgttaccaacggaacatcgacgtcgaacaaaattgtgggtatgtacgccgcgccatccggcagtacgctgttgatcgaccgcaattgtcataaatcgctggcgcatctgttgatgatgaacgatgtagtgccagtctggctgaaaccgacgcgtaatgcgttggggattcttggtgggatcccgcgccgtgaatttactcgcgacagcatcgaagagaaagtcgctgctaccacgcaagcacaatggccggttcatgcggtgatcaccaactccacctatgatggcttgctctacaacaccgactggatcaaacagacgctggatgtcccgtcgattcacttcgattctgcctgggtgccgtacacccattttcatccgatctaccagggtaaaagtggtatgagcggcgagcgtgttgcgggaaaagtgatcttcgaaacgcaatcgacccacaaaatgctggcggcgttatcgcaggcttcgctgatccacattaaaggcgagtatgacgaagaggcctttaacgaagcctttatgatgcataccaccacctcgcccagttatcccattgttgcttcggttgagacggcggcggcgatgctgcgtggtaatccgggcaaacggctgattaaccgttcagtagaacgagctctgcattttcgcaaagaggtccagcggctgcgggaagagtctgacggttggtttttcgatatctggcaaccgccgcaggtggatgaagccgaatgctggcccgttgcgcctggcgaacagtggcacggctttaacgatgcggatgccgatcatatgtttctcgatccggttaaagtcactattttgacaccggggatggacgagcagggcaatatgagcgaggaggggatcccggcggcgctggtagcaaaattcctcgacgaacgtgggatcgtagtagagaaaaccggcccttataacctgctgtttctctttagtattggcatcgataaaaccaaagcaatgggattattgcgtgggttgacggaattcaaacgctcttacgatctcaacctgcggatcaaaaatatgctacccgatctctatgcagaagatcccgatttctaccgcaatatgcgtattcaggatctggcacaagggatccataagctgattcgtaaacacgatcttcccggtttgatgttgcgggcattcgatactttgccggagatgatcatgacgccacatcaggcatggcaacgacaaattaaaggcgaagtagaaaccattgcgctggaacaactggtcggtagagtatcggcaaatatgatcctgccttatccaccgggcgtaccgctgttgatgcctggagaaatgctgaccaaagagagccgcacagtactcgattttctactgatgctttgttccgtcgggcaacattaccccggttttgaaacggatattcacggcgcgaaacaggacgaagacggcgtttaccgcgtacgagtcctaaaaatggcgggataa

SEQ ID NO: 158 - protein, 713 aa, Escherichia coli
MNIIAIMGPHGVFYKDEPIKELESALVAQGFQIIWPQNSVDLLKFIEHNPRICGVIFDWDEYSLDLCSDINQLNEYLPLYAFINTHSTMDVSVQDMRMALWFFEYALGQAEDIAIRMRQYTDEYLDNITPPFTKALFTYVKERKYTFCTPGHMGGTAYQKSPVGCLFYDFFGGNTLKADVSISVTELGSLLDHTGPHLEAEEYIARTFGAEQSYIVTNGTSTSNKIVGMYAAPSGSTLLIDRNCHKSLAHLLMMNDVVPVWLKPTRNALGILGGIPRREFTRDSIEEKVAATTQAQWPVHAVITNSTYDGLLYNTDWIKQTLDVPSIHFDSAWVPYTHFHPIYQGKSGMSGERVAGKVIFETQSTHKMLAALSQASLIHIKGEYDEEAFNEAFMMHTTTSPSYPIVASVETAAAMLRGNPGKRLINRSVERALHFRKEVQRLREESDGWFFDIWQPPQVDEAECWPVAPGEQWHGFNDADADHMFLDPVKVTILTPGMDEQGNMSEEGIPAALVAKFLDERGIVVEKTGPYNLLFLFSIGIDKTKAMGLLRGLTEFKRSYDLNLRIKNMLPDLYAEDPDFYRNMRIQDLAQGIHKLIRKHDLPGLMLRAFDTLPEMIMTPHQAWQRQIKGEVETIALEQLVGRVSANMILPYPPGVPLLMPGEMLTKESRTVLDFLLMLCSVGQHYPGFETDIHGAKQDEDGVYRVRVLKMAG

SEQ ID NO: 159 - DNA, 1590 bp, Escherichia coli
atgaacaataacgatctggtcgcgaagctgtggaagctgtgcgacaacctgcgcgatggcggcgtttcctatcaaaactacgtcaatgaactcgcctcgctgctgtttttgaaaatgtgtaaagagaccggtcaggaagcggaatacctgccggaaggttaccgctgggatgacctgaaatcccgcatcggccaggagcagttgcagttctaccgaaaaatgctcgtgcatttaggcgaagatgacaaaaagctggtacaggcagtttttcataatgttagtaccaccatcaccgagccgaaacaaataaccgcactggtcagcaatatggattcgctggactggtacaacggcgcgcacggtaagtcgcgcgatgacttcggcgatatgtacgaagggctgttgcagaagaacgcgaatgaaaccaagtctggtgcaggccagtacttcaccccgcgtccgctgattaaaaccattattcatctgctgaaaccgcagccgcgtgaagtggtgcaggacccggcggcaggtacggcgggctttttgattgaagccgaccgctatgttaagtcgcaaaccaatgatctggacgaccttgatggcgacacgcaggatttccagatccaccgcgcgtttatcggcctcgaactggtgcccggcacccgtcgtctggcactgatgaactgcctgctgcacgatattgaaggcaacctcgaccacggcggcgcaatccgtctgggcaacactctgggtagcgacggtgaaaacctgccgaaggcgcatattgtcgccactaacccgccgtttggcagcgccgcaggcaccaacattacccgcacctttgttcacccgaccagcaacaaacagttgtgctttatgcagcatattatcgaaacgctgcatcccggcggtcgtgcggcggtggtggtgccggataacgtgctgtttgaaggcggcaaaggcaccgacattcgtcgtgacctgatggataagtgtcatctgcacaccattctgcgtctgccgaccggtattttttacgctcagggcgtgaagaccaacgtgctgttctttaccaaagggacggtggcgaacccgaatcaggataagaactgtaccgatgatgtgtgggtgtatgacctgcgtaccaatatgccgagtttcggcaagcgcacaccgtttaccgacgagcatttgcagccgtttgagcgcgtgtatggcgaagacccgcacggtttaagcccgcgcactgaaggtgaatggagttttaacgccgaagagacggaagttgccgacagcgaagagaacaaaaacaccgaccagcatcttgctaccagccgctggcgcaagttcagccgtgagtggatccgcaccgcaaaatccgattcgctggatatctcctggctgaaagataaagacagtattgatgccgacagcctgccggagccggatgtattagcggcagaagcgatgggcgaactggtacaggcgctgtctgaactggatgcgctgatgcgtgaactgggggcgagcgatgaggccgatttgcagcgtcagttgctggaagaagcgtttggtggggtgaaggaataa

SEQ ID NO: 160 - protein, 529 aa, Escherichia coli
MNNNDLVAKLWKLCDNLRDGGVSYQNYVNELASLLFLKMCKETGQEAEYLPEGYRWDDLKSRIGQEQLQFYRKMLVHLGEDDKKLVQAVFHNVSTTITEPKQITALVSNMDSLDWYNGAHGKSRDDFGDMYEGLLQKNANETKSGAGQYFTPRPLIKTIIHLLKPQPREVVQDPAAGTAGFLIEADRYVKSQTNDLDDLDGDTQDFQIHRAFIGLELVPGTRRLALMNCLLHDIEGNLDHGGAIRLGNTLGSDGENLPKAHIVATNPPFGSAAGTNITRTFVHPTSNKQLCFMQHIIETLHPGGRAAVVVPDNVLFEGGKGTDIRRDLMDKCHLHTILRLPTGIFYAQGVKTNVLFFTKGTVANPNQDKNCTDDVWVYDLRTNMPSFGKRTPFTDEHLQPFERVYGEDPHGLSPRTEGEWSFNAEETEVADSEENKNTDQHLATSRWRKFSREWIRTAKSDSLDISWLKDKDSIDADSLPEPDVLAAEAMGELVQALSELDALMRELGASDEADLQRQLLEEAFGGVKE

SEQ ID NO: 161 - DNA, 918 bp, Saccharomyces cerevisiae
atggttgaggaaaattccagagttttgattgttcttccttatacaccgcctagtgctactttgcagaggattatagggcaaactattccgttcttaagagaatgtcaaagtcaactagacatcgtgattgtacctgaattcaaaacctcattccagttggattctgcgctagggaagatgtacagtattaccagggatgtccttttgggctatggaatgatcaacagcggaatcaacatcatattcaacaatattcatttcgtcgagagtaatttgcaatggaaagtggttttattgccacaggaatccacttttgaaacttggaagctagagttgggacaaggacaataccatagtatagaacattatgcattacacgataatataatggaagagatagaaggtcccaaagatgctaacaaatttcatgtcaccgcattgggcggaacgttcgaccacattcacgatggacataaaatattgttgagcgtctctacattcatcacgtcacaaaggttaatttgtggaattacgtgcgatgagctcttgcaaaacaagaaatacaaagagttgattgaaccttatgatacacgatgcaggcacgtacatcaattcatcaagttgttaaaaccggatctctccgtagaactagttcccttaagggacgtgtgcggccccacagggaaagtacccgagatagaatgtttagttgtgagtagagaaaccgtcagtggggcagagactgtgaataagactaggattgaaaaaggcatgagcccattggcagtacatgtggttaatgtacttggaggaagggaggaagacggctggagcgagaagttaagcagcacggaaatcagacgcctacttaagtcctctgcttcgccaacgtgcactccacaaaacccttgcgtataa

SEQ ID NO: 162 - protein, 305 aa, Saccharomyces cerevisiae
MVEENSRVLIVLPYTPPSATLQRIIGQTIPFLRECQSQLDIVIVPEFKTSFQLDSALGKMYSITRDVLLGYGMINSGINIIFNNIHFVESNLQWKVVLLPQESTFETWKLELGQGQYHSIEHYALHDNIMEEIEGPKDANKFHVTALGGTFDHIHDGHKILLSVSTFITSQRLICGITCDELLQNKKYKELIEPYDTRCRHVHQFIKLLKPDLSVELVPLRDVCGPTGKVPEIECLVVSRETVSGAETVNKTRIEKGMSPLAVHVVNVLGGREEDGWSEKLSSTEIRRLLKSSASPTCTPQNPCV